(19) United States
(12) Patent Application Publication
(10) Pub. No.: US 2024/0165056 A1
(43) Pub. Date: May 23, 2024

(54) USE OF TAMOXIFEN AND RELATED COMPOUNDS TO REDUCE BREAST IMPLANT CAPSULE FORMATION AND CAPSULAR CONTRACTURE

(71) Applicants: Ohio State Innovation Foundation, Columbus, OH (US); RESEARCH INSTITUTE AT NATIONWIDE CHILDREN'S HOSPITAL, Columbus, OH (US)

(72) Inventors: Jenny BARKER, Powell, OH (US); Kevin BLUM, Columbus, OH (US)

(21) Appl. No.: 18/281,115

(22) PCT Filed: Mar. 11, 2022
(86) PCT No.: PCT/US2022/019881

§ 371 Date: Sep. 8, 2023

Related U.S. Application Data
(60) Provisional application No. 63/159,752, filed on Mar. 11, 2021.

(51) Int. Cl. A61K 31/138; A61L 27/18; A61L 27/54

(52) U.S. Cl. CPC A61K 31/138; A61L 27/18; A61L 27/54; A61L 2300/204; A61L 2300/412

(57) ABSTRACT

Disclosed are methods for treating or preventing capsular contracture or capsule formation in a subject with an implant, the method comprising administering to the subject tamoxifen or a metabolite thereof in a localized form. Also disclosed is a surgical implant coated with tamoxifen or a metabolite thereof.

### CROSS-REFERENCE TO RELATED APPLICATIONS

This application claims benefit of U.S. Provisional Application No. 63/159,752, filed Mar. 11, 2021, herein incorporated by reference in its entirety.

### BACKGROUND

The formation of a capsule is a common foreign body reaction to all implanted materials, and the degree of capsule formation varies across patients, implant types, and procedure types. Capsules formed in response to foreign bodies are predominantly made up of inflammatory cells, fibroblasts, myofibroblasts, and collagen. (Bui 2015; Anderson 2008; Segreto 2018). For breast implant capsules in particular, capsular contracture is a clinically significant diagnosis that results in pain, deformation of the implant, and need for surgical reoperation, occurring in 8-15% of patients (Adams 2006). Capsular contracture is one of the most common causes for reoperation following breast reconstruction after cancer (Shin 2018). Capsular contracture is classified according to Baker grading from Ito IV, with III and IV being more severe and clinically significant (Speak 1995). Factors associated with the development of capsular contracture are time from implantation, with an increasing rate of contracture with time, and thickness of the fibrous capsule that forms around the implant (Bui 2015). Risk factors for capsular contracture include radiation exposure, implant contamination or infection, smooth-walled implants, and subglandular placement of the implant as opposed to subpectoral placement; however, the primary cause of capsular contracture remains idiopathic in nature and it is unclear why some patients, but not others, develop clinically significant capsular contracture in comparable settings (Wan 2016; Mempin 2018; Haran 2021).

Options for treatment of breast implant capsular contracture include surgical capsulectomy, implant exchange (removal and replacement with a new implant), and altering the position where the implant is placed (i.e., from a subglandular plane to a subpectoral plane or in an adjacent “neopocket”) (Wan 2016). Emphasis is aimed at primary prevention of capsular contracture through modification of the surgical method, implant location, strategies to reduce the likelihood of implant contamination, and the use of textured implant surfaces (Wan 2016; Headon 2015). Textured surfaces have been shown to decrease the formation of an implant capsule, with a proposed mechanism of decreasing collagen and cellular alignment in the fibrous capsule (Fisher 2015). However, recent studies have found incidence of breast implant associated lymphomas following textured surface implantation through a currently unknown mechanism (Coleman 1991; Hwang 2015; Chen 2020). Surface modifications and addition of antibiotic and antifibrotic drugs have shown some success in preventing capsular contracture (Shin 2018; Mempin 2018). Antibiotics are utilized to address the biofilm hypothesis of capsule formation, which posits that capsule formation and contracture are exacerbated by the presence of a bacterial biofilm around the implant, which leads to long-term inflammation (Mempin 2018; Ajdic 2014). Additionally, recent research has focused on immune modulation on the formation of capsular contracture. TGF-β and its role in the development and function of myofibroblasts has been implicated (Persichetti 2014). In previous studies, knock-out of periostin, an integrin ligand, in mice was shown to decrease the inflammatory markers as well as markers of fibrosis, including αSMA and collagen I (Bae 2018). In addition, estrogen signaling has been noted to play a role in capsule formation, particularly with higher amounts of estrogen increasing the presence of myofibroblasts and collagen surrounding implants. Correspondingly, capsular contracture is less common in patients on anti-estrogen therapy (Persichetti 2014).

Tamoxifen, a selective estrogen receptor modulator, is clinically indicated in estrogen receptor positive breast cancer treatment for which it has a potent antitumor effect (Kelley 2012). However, tamoxifen has previously shown off-target efficacy in the reduction of fibrosis for skin scar formation in humans, particularly in terms of decreasing the formation of hypertrophic scars in vivo, and decreasing human fibroblast contraction of collagen matrices in vitro (Mousavi 2010; Payne 2006; Gragnani 2010). Tamoxifen has also been shown to reduce renal fibrosis after kidney injury in a rat model (Delle 2012).

What is needed in the art are methods of estrogen modulation to treat and prevent capsular contracture rates and fibrotic tissue formation.

### SUMMARY

Disclosed herein is a method for treating or preventing capsular contracture or capsule formation in a subject with an implant, the method comprising administering to the subject tamoxifen or a metabolite thereof in a localized form. Also disclosed herein is a medical device conjugated to tamoxifen or a derivative, formulation, or metabolite thereof. The tamoxifen, metabolite, derivative, or formulation thereof can be used with an adjuvant, carrier, or formulation for delivery.

The implant can be a breast implant; artificial joint or limb; cardiac pacemaker; coronary stent; implantable cardioverter defibrillator; cerebral spinal fluid shunt system; cochlear implant or ear tube; surgical mesh implant; screws, rods, or artificial discs; intrauterine device; artificial eye lens. The metabolite, derivative, or formulation of tamoxifen can be endoxifen, N-desmethyl-tamoxifen, or Z-4-hydroxy-tamoxifen, or a combination thereof. The tamoxifen or formulation, derivative, or metabolite thereof can be combined with other treatment regimens, drugs, or methods of treatment or prevention. In one example, the implant can be soaked in the metabolite, derivative, or formulation. The tamoxifen, derivative, formulation, or metabolite thereof can be conjugated to the implant. The tamoxifen, derivative, formulation, or metabolite thereof can be administered in a gel form. The tamoxifen, derivative, formulation, or metabolite thereof can be provided in slow-release form. The tamoxifen, derivative, formulation, or metabolite thereof can be provided in an amount which is less or more than the amount given to treat or prevent breast cancer.

### DESCRIPTION OF DRAWINGS

FIG. 1A-F shows experimental design 6 mm diameter silicone implants. (A) were implanted under the panniculus carnosus muscle (B) bilaterally (C) and explanted after 2 weeks. Implants and surrounding tissue were harvested en bloc (D) and sectioned for histology (E) scale bar 500 μm. F) Mouse weight in control animals and tamoxifen treated animals over the experimentation time. Data compared with unpaired t-tests or Mann-Whitney tests as appropriate (see text). * p<0.05, ** p<0.01, *** p<0.001.

FIG. 2A-F shows tamoxifen decreases capsule thickness and collagen deposition. A) Representative H&E staining demonstrating the anatomical layers surrounding the implant, including the fibrous capsule, panniculus carnosus muscle (PCM), and subcutaneous fat. Double sided arrow demonstrates the thickness of the capsule. B) average thickness of the fibrous capsule at 2 weeks. C) Average total cellularity of the capsules. D) Representative picrosirius red images of the capsules. Total collagen (E) and area fraction of collagen (F). Scale bars=100 μm. Data compared with unpaired t-tests or Mann-Whitney tests as appropriate (see text). * p<0.05, ** p<0.01, *** p<0.001.

FIG. 3A-I shows estrogen receptors and proliferation. A) Representative histology of estrogen receptor α, quantified as cellular fraction (B) and total positive cells per mm (C). D) Representative histology of estrogen receptor β, quantified as cellular fraction (E) and total positive cells per mm (F). G) Representative histology of proliferative marker Ki-67, quantified as cellular fraction (H) and total positive cells per mm (I). Scale bars=50 μm. Data compared with unpaired t-tests or Mann-Whitney tests as appropriate (see text). * p<0.05, ** p<0.01, *** p<0.001.

FIG. 4A-F shows αSMA and CD31 expression. A) Representative histology of myofibroblast marker αSMA, quantified as cellular fraction (B) and total positive cells per mm (C). D) Representative histology of endothelial cell marker CD31, quantified as vessels per mm2 (E) and vessels per mm (F). Scale bars=50 μm. Data compared with unpaired t-tests or Mann-Whitney tests as appropriate (see text). * p<0.05, ** p<0.01, *** p<0.001.

FIG. 5A-I shows macrophage response. A) Representative histology of macrophage marker CD68, quantified as cellular fraction (B) and total positive cells per mm (C). Representative imaging of proinflammatory macrophage marker iNOS (D) and anti-inflammatory macrophage marker CD163 (E) also shown, with quantifications performed as cellular fraction (F and G) and as total cells per mm (H and I) for iNOS and CD163, respectively. Scale bars=50 μm. Data compared with unpaired t-tests or Mann-Whitney tests as appropriate (see text). * p<0.05, ** p<0.01, *** p<0.001.

FIG. 6 shows the effects of beige mouse model on capsule formation quantified data for all experimental groups in study including control, tamoxifen treated, and beige mice. Data for weight as well as all histological markers examined are included as presented in the main text. Data compared with unpaired t-tests or Mann-Whitney tests as appropriate (see text). * p<0.05, ** p<0.01, *** p<0.001.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Association” refers to a state wherein two items are physically connected together, so that to transport one item would necessarily transport some or all of the second item. For example, an implant may be associated with a composition, so that inserting the implant into a patient will necessarily insert into that patient some or all of a composition that has been associated with the implant. “Host”, “Person”, “Subject”, “Patient” and the like are used synonymously to refer to the living being into which a device of the present disclosure is implanted.

“Implanted” refers to having completely or partially placed a foreign object or device within a host. A device is partially implanted when some of the device reaches, or extends to the outside of, a host.

“Inhibit capsule”, “reduce capsular formation” and the like are used synonymously to refer to the action of agents or compositions which result in a statistically significant decrease in the formation of capsular formation that can be expected to occur in the absence of the agent or composition.

“Inhibitor” refers to an agent which prevents a biological process from occurring or slows the rate or degree of occurrence of a biological process.

“Derivative” or “metabolite” refers to a chemically or biologically modified version of a chemical compound that is structurally similar to a parent compound and (actually or theoretically) derivable from that parent compound/or from which the parent compound is derived. A metabolite may have different chemical or physical properties of the parent compound. For example, the derivative or metabolite may be more hydrophilic or it may have altered reactivity as compared to the parent compound. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds that simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

“Medical Device”, “Implant”, “Medical Device or Implant”, “implant/device” and the like are used synonymously to refer to any object that is designed to be placed partially or wholly within a patient's body for one or more therapeutic or prophylactic purposes such as for restoring physiological function, alleviating symptoms associated with disease, delivering therapeutic agents, and/or repairing or replacing or augmenting etc. damaged or diseased organs and tissues. While normally composed of biologically compatible synthetic materials (e.g., medical-grade stainless steel, titanium and other metals; polymers such as polyurethane, silicon, PLA, PLGA and other materials) that are exogenous, some medical devices and implants include materials derived from animals (e.g., “xenografts” such as whole animal organs; animal tissues such as heart valves; naturally occurring or chemically-modified molecules such as collagen, hyaluronic acid, proteins, carbohydrates and others), human donors (e.g., “allografts” such as whole organs; tissues such as bone grafts, skin grafts and others), or from the patients themselves (e.g., “autografts” such as saphenous vein grafts, skin grafts, tendon/ligament/muscle transplants). Representative medical devices of particular utility in the present disclosure include, but are not restricted to, vascular stents, gastrointestinal stents, tracheal/bronchial stents, genital-urinary stents, ENT stents, drug delivery balloons and catheters, hemodialysis access devices, vascular grafts, anastomotic connector devices, surgical sheets (e.g., films or meshes), soft tissue implants (such as breast implants, facial implants, tissue fillers, aesthetic implants and the like), implantable electrodes (cardiac pacemakers, neurostimulation devices), implantable sensors, drug delivery pumps, anti-adhesion barriers, and shunts.

“Release of an agent” refers to a statistically significant presence of the agent, or a subcomponent thereof, which has disassociated from the implant/device.

### Description of Invention

Capsular contracture is a response of the immune system to foreign materials in the human body. It can occur in context of the complications from breast implants and artificial joint prosthetics. The occurrence of capsular contraction follows the formation of capsules of tightly-woven collagen fibers, created by the immune response to the presence of foreign objects surgically installed to the human body, e.g. breast implants, artificial pacemakers, orthopedic prostheses; biological protection by isolation and toleration. Capsular contracture occurs when the collagen-fiber capsule shrinks, tightens and compresses the breast implant. It is a medical complication that can be painful and discomfortable, and can distort the aesthetics of the breast implant and the breast.

Moreover, because capsular contracture is a consequence of the immune system defending the patient's bodily integrity and health, it might reoccur, even after the requisite corrective surgery for the initial incidence. The degree of an incidence of capsular contracture is graded using the four-grade Baker scale:
Grade I—the breast is normally soft and appears natural in size and shape
Grade II—the breast is a little firm, but appears normal
Grade III—the breast is firm and appears abnormal
Grade IV—the breast is hard, painful to the touch, and appears abnormal

Disclosed herein is a method of treating, preventing or reducing formation of an abnormal capsule formation on or near a foreign object in a subject. These methods can help prevent capsule formation, particularly capsular contracture, from ever forming in the subject. These methods can also treat, or reduce the severity of, capsule formation in patients where a capsule or capsular contracture has already formed. When the capsular contracture has already formed, the methods disclosed herein can reduce a Grade IV contracture to a Grade I, II, or III, or significantly reduce it, or eliminate it completely. Likewise, it can reduce a Grade III contracture to a Grade I or II, or significantly reduce it or eliminate it completely. Similarly, it can reduce a Grade II contracture to a Grade I, or significantly reduce it or eliminate it completely. Similarly, it can reduce or eliminate a Grade I contracture. What is meant by “reduce” is discussed in more detail below.

Specifically, a method of treating or reducing the severity of capsule formation can comprise administering to the subject tamoxifen or a derivative, formulation, or metabolite thereof, wherein said tamoxifen or derivative, formulation, or metabolite thereof is administered in a localized form on or near the foreign object. The abnormal capsule formation can be a capsular contraction, or may be a capsule which is not classified on the Baker scale as being a capsular contracture yet.

Also disclosed herein is a surgical implant wherein said tamoxifen, or a metabolite, derivative, or formulation thereof is conjugated to the surgical implant. Such implants are discussed in more detail below.

As stated above, capsular contracture can be treated or prevented by the administration of tamoxifen or a derivative, formulation, or metabolite thereof. Tamoxifen, or a salt (also called a “pharmaceutically acceptable salt”) or derivative thereof, is an “anti-estrogen,” a selective estrogen-receptor modulator. Anti-estrogens such as tamoxifen have been shown to dramatically reduce the risk of breast cancer (Powles, T. J., Nat Rev Cancer, 2:787-794, 2002) and of breast cancer recurrence (Jordan, V. C., Nat Rev Drug Discov, 2:205-213, 2003). Tamoxifen is a partial agonist and exhibits both species and tissue specificity for inducing either an agonist or antagonist response. In both rats and humans, tamoxifen exhibits partial agonism, e.g., producing antagonist effects in the breast, but agonist effects in the vagina and endometrium. Long-term tamoxifen use has been associated with a reduced incidence of contralateral breast cancer (antagonist), a reduced incidence of primary breast cancer in high-risk women (antagonist), maintenance of bone density (agonist), and increased risk of endometrial carcinomas (agonist). The structure of tamoxifen is:

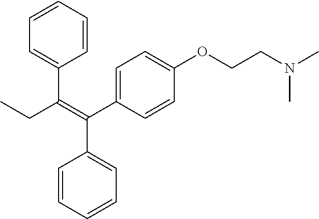 [SMILES: CC/C(=C(\c1ccccc1)c1ccc(OCCN(C)C)cc1)c1ccccc1]

Derivatives of tamoxifen are known in the art, see for example, U.S. Patent Application Publication No. 2016/0075726, U.S. Patent Application Publication No. 2006/0105041 and U.S. Patent Application Publication No. 2004/0138314, which are incorporated herein by reference. The biochemical mechanism of action of tamoxifen in the treatment of breast cancer is widely understood to involve two active metabolites, 4-hydroxy-N-desmethyl-tamoxifen (endoxifen) and Z-4-hydroxy-tamoxifen (4HT).

In some embodiments, said metabolites comprise any one of, or a combination of, tamoxifen, norendoxifen, endoxifen, 4′-Hydroxy Tamoxifen, Z-4-Hydroxy Tamoxifen, N-Desmethyl-4′-Hydroxy Tamoxifen, and N-Desmethyl Tamoxifen.

U.S. Pat. No. 5,219,549, incorporated herein by reference, discloses tamoxifen derivatives wherein the alkyl chain of the molecule is substituted with fluorine or iodine, such as fluorotamoxifen. The compound 4-hydroxy tamoxifen (afimoxifene), or 1-[4-(2-N-dimethylaminoethoxy)-phenyl]-1-(4-hydroxyphenyl)-2-phenylbut-1-(Z)-ene, constitutes an active metabolite of the well characterized anti-estrogen compound, tamoxifen. Both cis and trans isomers exist, either of which, alone or in combination, are useful. Methods for preparing 4-hydroxy tamoxifen are well known. For example, U.S. Pat. No. 4,919,937, incorporated herein by reference, discloses a synthesis derived methods that occurs in several stages: Stage 1—Reaction between 4-(β-dimethylaminoethoxy)-α-ethyldeoxybenzoin and p-(2-tetrahydropyranyloxy)phenylmagnesium bromide; Stage 2—Separately from stage 1, formation of 1-(4-hydroxyphenyl)-2-phenyl-1-butanone by hydroxylation of 1,2-diphenyl-1-butanone; Stage 3—Reaction between the products of stages 1 and 2 to form 1-(4-dimethylaminoethoxyphenyl)[4p-2-tetrahydropyranyloxy)phenyl]-2-phe-nylbutan-1-ol; Stage 4—Dehydration with methanol/hydrochloric acid produces 1-[p-(β-dimethylaminoethoxy)phenyl]-trans-1-(p-hydroxypheny-1)-2-pheny-1-but-1-ene=4-OH-tamoxifen, a mixture of cis and trans isomers; Stage 5—Separation of the cis and trans isomers by chromatography and crystallization to constant specific activity. Suitable doses are disclosed for example, in U.S. Pat. No. 7,485,623, which is incorporated herein by reference.

Also contemplated herein are other selective estrogen receptor modulators (SERMs) which, like tamoxifen, can be used to treat or prevent capsular contracture. Raloxifene ([6-hydroxy-2-(4-hydroxyphenyl)-benzothiophen-3-yl]-[4-[2-(1-piperidyl)ethoxy]phenyl]-methanone), shown below, is also of use in the methods disclosed herein. The structure of raloxifene is shown below:

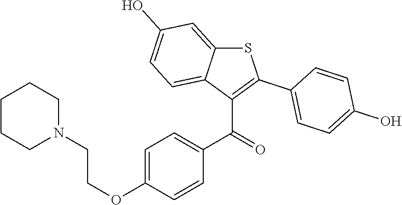 [SMILES: O=C(c1ccc(OCCN2CCCCC2)cc1)c1c(-c2ccc(O)cc2)sc2cc(O)ccc12]

Also contemplated is bazedoxifene (1-{4-[2-(Azepan-1-yl)ethoxy]benzyl}-2-(4-hydroxyphenyl)-3-methyl-1H-indol-5-ol), a third generation SERM developed by Pfizer; the structure is shown below:

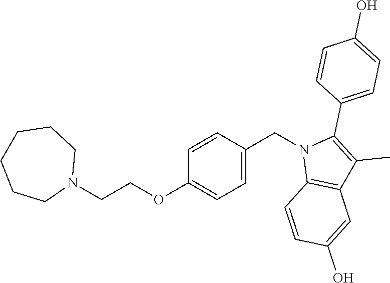 [SMILES: Cc1c(-c2ccc(O)cc2)n(Cc2ccc(OCCN3CCCCCC3)cc2)c2ccc(O)cc12]

Also contemplated is bazedoxifene, which has been approved for use in postmenopausal osteoporosis, and has been used for treatment of dyspareunia, breast cancer, and pancreatic cancer. Bazedoxifene is also of use in the methods disclosed herein. Further contemplated is arzoxifene (2-(4-Methoxyphenyl)-3-[4-(2-piperidin-1-ylethoxy)phenoxy]-1-benzothiophen-6-ol), a SERM that is a potent estrogen antagonist in mammary and uterine tissue while acting as an estrogen agonist to maintain bone density and lower serum cholesterol. Arzoxifene is also of use in the methods disclosed herein. The structure of arzoxifene is shown below:

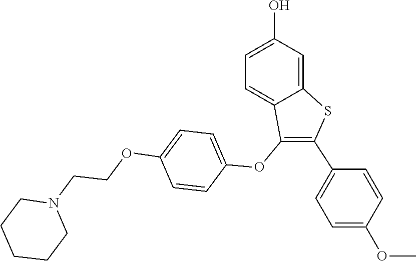 [SMILES: COc1ccc(-c2sc3cc(O)ccc3c2Oc2ccc(OCCN3CCCCC3)cc2)cc1]

Desmethylarzoxifene (2-(4-hydroxyphenyl)-3-[4-(2-piperidin-1-ylethoxy)phenoxy]-1-benzothiophen-6-ol) is also contemplated. Desmethylarzoxifene is also a SERM and is of use in the methods disclosed herein. Desmethylarzoxifene blocks estrogen-induced malignant transformation of human breast epithelial cell lines (Kastrati et al., PLOS One 6(11): e27876, 2011). The structure of desmethylarzoxifene is shown below:

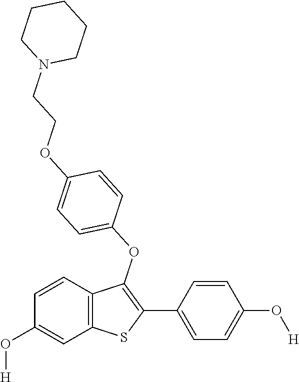 [SMILES: Oc1ccc(-c2sc3cc(O)ccc3c2Oc2ccc(OCCN3CCCCC3)cc2)cc1]

As discussed above, tamoxifen or a derivative, formulation, or metabolite thereof can reduce the size of an abnormal capsule formation. This reduction can be by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% as compared to a control, such as a subject who has not received tamoxifen or a derivative, formulation, or metabolite thereof.

For example, tamoxifen or a derivative, formulation, or metabolite thereof can reduce the duration of time until resolution of an abnormal capsule formation. This reduction in time can be by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% as compared to a control, such as a subject who has not received tamoxifen or a derivative, formulation, or metabolite thereof. Put another way, the reduction in time until resolution of capsule formation can be by 12, 24, 36, or 48 hours, or any amount in between, above, or below, or can be within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or can be within 1, 2, 3, 4, 5, 6, 7, 8, 9, or 10 years, or any amount below, above, or in-between these amounts.

Implantable or insertable medical devices (also referred to herein as implants) which can be constructed in accordance with the invention vary widely and include, for example, stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts), vascular access ports, dialysis ports, catheters (e.g., urological catheters or vascular catheters such as balloon catheters and various central venous catheters), guide wires, filters (e.g., vena cava filters and mesh filters for distil protection devices), embolization devices including cerebral aneurysm filler coils (including Guglielmi detachable coils and metal coils), cerebral spinal fluid shunt system, septal defect closure devices, drug depots that are adapted for placement in an artery for treatment of the portion of the artery distal to the device, myocardial plugs, pacemakers, leads including pacemaker leads, defibrillation leads, and coils, ventricular assist devices including left ventricular assist hearts and pumps, total artificial hearts, shunts, valves including heart valves and vascular valves, anastomosis clips and rings, cochlear implants, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, or other types of meshes, artificial ligaments, orthopedic prosthesis such as bone grafts, bone plates, fins and fusion devices, joint prostheses, such as artificial joint or limb implants, orthopedic fixation devices such as interference screws in the ankle, knee, and hand areas, tacks for ligament attachment and meniscal repair, rods and pins for fracture fixation, screws, discs, and plates for craniomaxillofacial repair or other types of repair, dental implants, or other devices that are implanted or inserted into the body.

The medical devices (or implants) of the present invention include, for example, implantable and insertable medical devices that are used for systemic diagnosis or treatment, as well as those that are used for the localized diagnosis or treatment of any mammalian tissue or organ. Non-limiting examples are tumors; organs including the heart, coronary and peripheral vascular system (referred to overall as “the vasculature”), the urogenital system, including kidneys, bladder, urethra, ureters, prostate, vagina, uterus and ovaries, eyes (such as eye implants, like artificial lenses), ears, spine, nervous system, lungs, trachea, esophagus, intestines, stomach, brain, liver and pancreas, skeletal muscle, smooth muscle, breast, dermal tissue, cartilage, tooth and bone.

Medical devices benefiting from the present invention thus include a variety of implantable and insertable medical devices including devices for insertion into and/or through a wide range of body lumens, for purposes of diagnosis, prevention, or treatment, several of which are recited above, including lumens of the cardiovascular system such as the heart and arteries (e.g., coronary, femoral, aorta, iliac, carotid and vertebro-basilar arteries) and veins. Examples include, but are not limited to, cardiac pacemakers, coronary stents, or implantable cardioverter defibrillators. Also contemplated are lumens of the genitourinary system such as the urethra (including prostatic urethra), bladder, ureters, vagina, uterus (such as intrauterine device), spermatic and fallopian tubes, the nasolacrimal duct, the eustachian tube, lumens of the respiratory tract such as the trachea, bronchi, nasal passages and sinuses, lumens of the gastrointestinal tract such as the esophagus, gut, duodenum, small intestine, large intestine, rectum, biliary and pancreatic duct systems, lumens of the lymphatic system, the major body cavities (peritoneal, pleural, pericardial) and so forth.

Also specifically contemplated herein are soft tissue implants. Examples include, but are not limited to, those used in the field of plastic surgery after breast amputations or in cosmetic surgery for breast augmentation. Further fields of application of these soft tissue implants are calf muscle prostheses or cheek, nose, gluteal muscle, testicular or brachial muscle implants.

Preferred subjects for the treatment, reduction, or prevention are vertebrate subjects, for example, humans, livestock and pets. Specifically contemplated are human patients who have or who are going to receive a surgical implant.

The tamoxifen or a derivative, formulation, or metabolite thereof can be administered in a localized form. This means that they can coat the outside of a porous or non-porous surface. In the case of a porous or soft surface, the tamoxifen or a derivative, formulation, or metabolite thereof can make up an outer layer of the device or implant. The tamoxifen or a derivative, formulation, or metabolite thereof is retained in the covering or coating of the foreign object, and in essence, provides for a topical application of the tamoxifen or a derivative, formulation, or metabolite thereof. The tamoxifen or a derivative, formulation, or metabolite thereof can be in the form of a gel or a slow-release formula. For example, U.S. Pat. No. 4,298,998 discloses that a coating is formed at a predetermined controlled distance from the surface of the implant, resulting in a coating at different locations. Also disclosed are medical grade gels as described in U.S. Pat. No. 4,944,749.

The tamoxifen or a derivative, formulation, or metabolite thereof is released to the surface of the foreign object and the body cavity in which the object is implanted. The drug or drugs mixture can be applied in a time-release fashion by adhering this mixture to a lattice or in a layered fashion that allows for controlled release from 1, 2, 3, 4, 5, 6, 7, 8, 9, 10, 11, 13, or 14 days, or 1, 2, 3, 4, 5, 6, 7, 8, 9, 10, 11, or 12 weeks, or 1, 2, 3, 4, 5, 6, 7, 8, 9, 10, 11, 12, 13, 14, 15, 16, 17, 18, 19, 20, 21, 22, 23, or 24, months, or 1, 2, 3, 4, 5, 6, 7, 8, 9, or 10 years or more. Further, tamoxifen or a derivative, formulation, or metabolite thereof can be applied, for example, to a smooth walled implant, textured wall implant or dissolvable textured implant. Further, the tamoxifen or a derivative, formulation, or metabolite thereof can be designed to have three dimensional properties to create a textured or smooth surface.

In another embodiment, the tamoxifen or a derivative, formulation, or metabolite thereof can be given in a localized form near the foreign object, but in a separate implant or delivery device. For example, a shunt or other separate implant can be used to release the tamoxifen or a derivative, formulation, or metabolite thereof.

In yet another embodiment, the tamoxifen or a derivative, formulation, or metabolite thereof can be administered to the subject via an injection near the site of the implant. The tamoxifen or a derivative, formulation, or metabolite thereof can be administered to the subject before, during, or after the foreign object has been implanted in the subject. For example, the tamoxifen or a derivative, formulation, or metabolite thereof can be administered before or after an implant is implanted. This can be done by injection near the site of the implant, or via a shunt or stent. The tamoxifen or a derivative, formulation, or metabolite thereof can also be administered at the same time that the implant is implanted. In this case, the tamoxifen or a derivative, formulation, or metabolite thereof can coat the surface, as described herein, or the tamoxifen or a derivative, formulation, or metabolite thereof can be administered locally via injection or other local form of delivery.

In accordance with the invention, the tamoxifen or a derivative, formulation, or metabolite thereof can be given simultaneously with one or more other drugs or formulations. These include, but are not limited to, antibiotics, anti-inflammatory agents, fibrosis-inhibiting agents, anti-scarring agents, leukotriene inhibitors or antagonists, cell growth inhibitors, calcium channel blockers, or other agents effective to treat various conditions associated with implant surgery and recovery. Other drugs include, but are not limited to, acyclovir, cephradine, malphalen, procaine, ephedrine, adriamycin, daunomycin, plumbagin, atropine, quinine, digoxin, quinidine, biologically active peptides, cephradine, cephalothin, cis-hydroxy-L-proline, melphalan, penicillin V, nicotinic acid, chemodeoxycholic acid, chlorambucil and anti-neoplastic agents such as paclitaxel, sirolimus, 5-flurouracil and the like. Other drugs include those that act as angiogenensis inhibitors or inhibit various growth factors such as epidermal growth factor, PDGF, VEGF, FGF (fibroblast growth factor) and the like. These drugs include anti-growth factor antibodies (neutrophilin-1) and growth factor receptor-specific inhibitors such as endostatin. As used herein, the term “drug” or “drugs” is used to include all types of therapeutic agents, whether small molecules or large molecules such as proteins, nucleic acids and the like. The drugs of the invention can be used alone or in combination.

The tamoxifen or a derivative, formulation, or metabolite thereof can be conjugated to the foreign object such as an implant. For example, the tamoxifen or a derivative, formulation, or metabolite thereof can be in a polymer layer. Examples include, but are not limited to, a semicrystalline polymer or an amorphous polymer, or both. Also contemplated is a hydrophilic polymer layer, where a covalent bond forms between the surface of the implant or device. Crosslinking can be used to form bonds. Tamoxifen could also be eluted from a mesh, fiber mat, or other drug delivery device adjacent to the implant.

The tamoxifen or derivative, formulation, or metabolite thereof can be applied to the surface of the implant by soaking, for example. Examples of implant delivery systems are generally described in U.S. Pat. No. 4,356,166 to Peterson et al. and U.S. Pat. No. 4,976,962 to Bichon et al. Yet another technology combines the advantages of covalent drug attachment with liposome formation where the active ingredient is attached to highly ordered lipid films (known as HARs) via a peptide linker. Further description can be found in WO 97/36616 and U.S. Pat. No. 5,851,536 to Yager et al.

Importantly, the tamoxifen or a derivative, formulation, or metabolite thereof can be given in an amount which differs from that given for the treatment of cancer, such as breast cancer. Typically, it is given at a dose from about 20-40 mg/day in a systemic form, such as a pill. Contemplated herein is local delivery of tamoxifen to a site at or near the site of the implant. The dosage can be given via injection or elution from a coating or a stent or shunt. The dosage can be 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, 0.05, 0.051, 0.052, 0.053, 0.054, 0.055, 0.056, 0.057, 0.058, 0.059, 0.06, 0.061, 0.062, 0.063, 0.064, 0.065, 0.066, 0.067, 0.068, 0.069, 0.07, 0.071, 0.072, 0.073, 0.074, 0.075, 0.076, 0.077, 0.078, 0.079, 0.08, 0.081, 0.082, 0.083, 0.084, 0.085, 0.086, 0.087, 0.088, 0.089, 0.09, 0.091, 0.092, 0.093, 0.094, 0.095, 0.096, 0.097, 0.098, 0.099, 0.1, 0.101, 0.102, 0.103, 0.104, 0.105, 0.106, 0.107, 0.108, 0.109, 0.11, 0.111, 0.112, 0.113, 0.114, 0.115, 0.116, 0.117, 0.118, 0.119, 0.12, 0.121, 0.122, 0.123, 0.124, 0.125, 0.126, 0.127, 0.128, 0.129, 0.13, 0.131, 0.132, 0.133, 0.134, 0.135, 0.136, 0.137, 0.138, 0.139, 0.14, 0.141, 0.142, 0.143, 0.144, 0.145, 0.146, 0.147, 0.148, 0.149, 0.15, 0.151, 0.152, 0.153, 0.154, 0.155, 0.156, 0.157, 0.158, 0.159, 0.16, 0.161, 0.162, 0.163, 0.164, 0.165, 0.166, 0.167, 0.168, 0.169, 0.17, 0.171, 0.172, 0.173, 0.174, 0.175, 0.176, 0.177, 0.178, 0.179, 0.18, 0.181, 0.182, 0.183, 0.184, 0.185, 0.186, 0.187, 0.188, 0.189, 0.19, 0.191, 0.192, 0.193, 0.194, 0.195, 0.196, 0.197, 0.198, 0.199, 0.2, 0.201, 0.202, 0.203, 0.204, 0.205, 0.206, 0.207, 0.208, 0.209, 0.21, 0.211, 0.212, 0.213, 0.214, 0.215, 0.216, 0.217, 0.218, 0.219, 0.22, 0.221, 0.222, 0.223, 0.224, 0.225, 0.226, 0.227, 0.228, 0.229, 0.23, 0.231, 0.232, 0.233, 0.234, 0.235, 0.236, 0.237, 0.238, 0.239, 0.24, 0.241, 0.242, 0.243, 0.244, 0.245, 0.246, 0.247, 0.248, 0.249, 0.25, 0.251, 0.252, 0.253, 0.254, 0.255, 0.256, 0.257, 0.258, 0.259, 0.26, 0.261, 0.262, 0.263, 0.264, 0.265, 0.266, 0.267, 0.268, 0.269, 0.27, 0.271, 0.272, 0.273, 0.274, 0.275, 0.276, 0.277, 0.278, 0.279, 0.28, 0.281, 0.282, 0.283, 0.284, 0.285, 0.286, 0.287, 0.288, 0.289, 0.29, 0.291, 0.292, 0.293, 0.294, 0.295, 0.296, 0.297, 0.298, 0.299, 0.3, 0.301, 0.302, 0.303, 0.304, 0.305, 0.306, 0.307, 0.308, 0.309, 0.31, 0.311, 0.312, 0.313, 0.314, 0.315, 0.316, 0.317, 0.318, 0.319, 0.32, 0.321, 0.322, 0.323, 0.324, 0.325, 0.326, 0.327, 0.328, 0.329, 0.33, 0.331, 0.332, 0.333, 0.334, 0.335, 0.336, 0.337, 0.338, 0.339, 0.34, 0.341, 0.342, 0.343, 0.344, 0.345, 0.346, 0.347, 0.348, 0.349, 0.35, 0.351, 0.352, 0.353, 0.354, 0.355, 0.356, 0.357, 0.358, 0.359, 0.36, 0.361, 0.362, 0.363, 0.364, 0.365, 0.366, 0.367, 0.368, 0.369, 0.37, 0.371, 0.372, 0.373, 0.374, 0.375, 0.376, 0.377, 0.378, 0.379, 0.38, 0.381, 0.382, 0.383, 0.384, 0.385, 0.386, 0.387, 0.388, 0.389, 0.39, 0.391, 0.392, 0.393, 0.394, 0.395, 0.396, 0.397, 0.398, 0.399, 0.4, 0.401, 0.402, 0.403, 0.404, 0.405, 0.406, 0.407, 0.408, 0.409, 0.41, 0.411, 0.412, 0.413, 0.414, 0.415, 0.416, 0.417, 0.418, 0.419, 0.42, 0.421, 0.422, 0.423, 0.424, 0.425, 0.426, 0.427, 0.428, 0.429, 0.43, 0.431, 0.432, 0.433, 0.434, 0.435, 0.436, 0.437, 0.438, 0.439, 0.44, 0.441, 0.442, 0.443, 0.444, 0.445, 0.446, 0.447, 0.448, 0.449, 0.45, 0.451, 0.452, 0.453, 0.454, 0.455, 0.456, 0.457, 0.458, 0.459, 0.46, 0.461, 0.462, 0.463, 0.464, 0.465, 0.466, 0.467, 0.468, 0.469, 0.47, 0.471, 0.472, 0.473, 0.474, 0.475, 0.476, 0.477, 0.478, 0.479, 0.48, 0.481, 0.482, 0.483, 0.484, 0.485, 0.486, 0.487, 0.488, 0.489, 0.49, 0.491, 0.492, 0.493, 0.494, 0.495, 0.496, 0.497, 0.498, 0.499, 0.5, 0.501, 0.502, 0.503, 0.504, 0.505, 0.506, 0.507, 0.508, 0.509, 0.51, 0.511, 0.512, 0.513, 0.514, 0.515, 0.516, 0.517, 0.518, 0.519, 0.52, 0.521, 0.522, 0.523, 0.524, 0.525, 0.526, 0.527, 0.528, 0.529, 0.53, 0.531, 0.532, 0.533, 0.534, 0.535, 0.536, 0.537, 0.538, 0.539, 0.54, 0.541, 0.542, 0.543, 0.544, 0.545, 0.546, 0.547, 0.548, 0.549, 0.55, 0.551, 0.552, 0.553, 0.554, 0.555, 0.556, 0.557, 0.558, 0.559, 0.56, 0.561, 0.562, 0.563, 0.564, 0.565, 0.566, 0.567, 0.568, 0.569, 0.57, 0.571, 0.572, 0.573, 0.574, 0.575, 0.576, 0.577, 0.578, 0.579, 0.58, 0.581, 0.582, 0.583, 0.584, 0.585, 0.586, 0.587, 0.588, 0.589, 0.59, 0.591, 0.592, 0.593, 0.594, 0.595, 0.596, 0.597, 0.598, 0.599, 0.6, 0.601, 0.602, 0.603, 0.604, 0.605, 0.606, 0.607, 0.608, 0.609, 0.61, 0.611, 0.612, 0.613, 0.614, 0.615, 0.616, 0.617, 0.618, 0.619, 0.62, 0.621, 0.622, 0.623, 0.624, 0.625, 0.626, 0.627, 0.628, 0.629, 0.63, 0.631, 0.632, 0.633, 0.634, 0.635, 0.636, 0.637, 0.638, 0.639, 0.64, 0.641, 0.642, 0.643, 0.644, 0.645, 0.646, 0.647, 0.648, 0.649, 0.65, 0.651, 0.652, 0.653, 0.654, 0.655, 0.656, 0.657, 0.658, 0.659, 0.66, 0.661, 0.662, 0.663, 0.664, 0.665, 0.666, 0.667, 0.668, 0.669, 0.67, 0.671, 0.672, 0.673, 0.674, 0.675, 0.676, 0.677, 0.678, 0.679, 0.68, 0.681, 0.682, 0.683, 0.684, 0.685, 0.686, 0.687, 0.688, 0.689, 0.69, 0.691, 0.692, 0.693, 0.694, 0.695, 0.696, 0.697, 0.698, 0.699, 0.7, 0.701, 0.702, 0.703, 0.704, 0.705, 0.706, 0.707, 0.708, 0.709, 0.71, 0.711, 0.712, 0.713, 0.714, 0.715, 0.716, 0.717, 0.718, 0.719, 0.72, 0.721, 0.722, 0.723, 0.724, 0.725, 0.726, 0.727, 0.728, 0.729, 0.73, 0.731, 0.732, 0.733, 0.734, 0.735, 0.736, 0.737, 0.738, 0.739, 0.74, 0.741, 0.742, 0.743, 0.744, 0.745, 0.746, 0.747, 0.748, 0.749, 0.75, 0.751, 0.752, 0.753, 0.754, 0.755, 0.756, 0.757, 0.758, 0.759, 0.76, 0.761, 0.762, 0.763, 0.764, 0.765, 0.766, 0.767, 0.768, 0.769, 0.77, 0.771, 0.772, 0.773, 0.774, 0.775, 0.776, 0.777, 0.778, 0.779, 0.78, 0.781, 0.782, 0.783, 0.784, 0.785, 0.786, 0.787, 0.788, 0.789, 0.79, 0.791, 0.792, 0.793, 0.794, 0.795, 0.796, 0.797, 0.798, 0.799, 0.8, 0.801, 0.802, 0.803, 0.804, 0.805, 0.806, 0.807, 0.808, 0.809, 0.81, 0.811, 0.812, 0.813, 0.814, 0.815, 0.816, 0.817, 0.818, 0.819, 0.82, 0.821, 0.822, 0.823, 0.824, 0.825, 0.826, 0.827, 0.828, 0.829, 0.83, 0.831, 0.832, 0.833, 0.834, 0.835, 0.836, 0.837, 0.838, 0.839, 0.84, 0.841, 0.842, 0.843, 0.844, 0.845, 0.846, 0.847, 0.848, 0.849, 0.85, 0.851, 0.852, 0.853, 0.854, 0.855, 0.856, 0.857, 0.858, 0.859, 0.86, 0.861, 0.862, 0.863, 0.864, 0.865, 0.866, 0.867, 0.868, 0.869, 0.87, 0.871, 0.872, 0.873, 0.874, 0.875, 0.876, 0.877, 0.878, 0.879, 0.88, 0.881, 0.882, 0.883, 0.884, 0.885, 0.886, 0.887, 0.888, 0.889, 0.89, 0.891, 0.892, 0.893, 0.894, 0.895, 0.896, 0.897, 0.898, 0.899, 0.9, 0.901, 0.902, 0.903, 0.904, 0.905, 0.906, 0.907, 0.908, 0.909, 0.91, 0.911, 0.912, 0.913, 0.914, 0.915, 0.916, 0.917, 0.918, 0.919, 0.92, 0.921, 0.922, 0.923, 0.924, 0.925, 0.926, 0.927, 0.928, 0.929, 0.93, 0.931, 0.932, 0.933, 0.934, 0.935, 0.936, 0.937, 0.938, 0.939, 0.94, 0.941, 0.942, 0.943, 0.944, 0.945, 0.946, 0.947, 0.948, 0.949, 0.95, 0.951, 0.952, 0.953, 0.954, 0.955, 0.956, 0.957, 0.958, 0.959, 0.96, 0.961, 0.962, 0.963, 0.964, 0.965, 0.966, 0.967, 0.968, 0.969, 0.97, 0.971, 0.972, 0.973, 0.974, 0.975, 0.976, 0.977, 0.978, 0.979, 0.98, 0.981, 0.982, 0.983, 0.984, 0.985, 0.986, 0.987, 0.988, 0.989, 0.99, 0.991, 0.992, 0.993, 0.994, 0.995, 0.996, 0.997, 0.998, or 0.999 mg/day, or 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3, 3.01, 3.02, 3.03, 3.04, 3.05, 3.06, 3.07, 3.08, 3.09, 3.1, 3.11, 3.12, 3.13, 3.14, 3.15, 3.16, 3.17, 3.18, 3.19, 3.2, 3.21, 3.22, 3.23, 3.24, 3.25, 3.26, 3.27, 3.28, 3.29, 3.3, 3.31, 3.32, 3.33, 3.34, 3.35, 3.36, 3.37, 3.38, 3.39, 3.4, 3.41, 3.42, 3.43, 3.44, 3.45, 3.46, 3.47, 3.48, 3.49, 3.5, 3.51, 3.52, 3.53, 3.54, 3.55, 3.56, 3.57, 3.58, 3.59, 3.6, 3.61, 3.62, 3.63, 3.64, 3.65, 3.66, 3.67, 3.68, 3.69, 3.7, 3.71, 3.72, 3.73, 3.74, 3.75, 3.76, 3.77, 3.78, 3.79, 3.8, 3.81, 3.82, 3.83, 3.84, 3.85, 3.86, 3.87, 3.88, 3.89, 3.9, 3.91, 3.92, 3.93, 3.94, 3.95, 3.96, 3.97, 3.98, 3.99, 4, 4.01, 4.02, 30 4.03, 4.04, 4.05, 4.06, 4.07, 4.08, 4.09, 4.1, 4.11, 4.12, 4.13, 4.14, 4.15, 4.16, 4.17, 4.18, 4.19, 4.2, 4.21, 4.22, 4.23, 4.24, 4.25, 4.26, 4.27, 4.28, 4.29, 4.3, 4.31, 4.32, 4.33, 4.34, 4.35, 4.36, 4.37, 4.38, 4.39, 4.4, 4.41, 4.42, 4.43, 4.44, 4.45, 4.46, 4.47, 4.48, 4.49, 4.5, 4.51, 4.52, 4.53, 4.54, 4.55, 4.56, 4.57, 4.58, 4.59, 4.6, 4.61, 4.62, 4.63, 4.64, 4.65, 4.66, 4.67, 4.68, 4.69, 4.7, 4.71, 4.72, 4.73, 4.74, 4.75, 4.76, 4.77, 4.78, 4.79, 4.8, 4.81, 4.82, 4.83, 4.84, 4.85, 4.86, 4.87, 4.88, 4.89, 4.9, 4.91, 4.92, 4.93, 4.94, 4.95, 4.96, 4.97, 4.98, 4.99, 5, 5.01, 5.02, 5.03, 5.04, 5.05, 5.06, 5.07, 5.08, 5.09, 5.1, 5.11, 5.12, 5.13, 5.14, 5.15, 5.16, 5.17, 5.18, 5.19, 5.2, 5.21, 5.22, 5.23, 5.24, 5.25, 5.26, 5.27, 5.28, 5.29, 5.3, 5.31, 5.32, 5.33, 5.34, 5.35, 5.36, 5.37, 5.38, 5.39, 5.4, 5.41, 5.42, 5.43, 5.44, 5.45, 5.46, 5.47, 5.48, 5.49, 5.5, 5.51, 5.52, 5.53, 5.54, 5.55, 5.56, 5.57, 5.58, 5.59, 5.6, 5.61, 5.62, 5.63, 5.64, 5.65, 5.66, 5.67, 5.68, 5.69, 5.7, 5.71, 5.72, 5.73, 5.74, 5.75, 5.76, 5.77, 5.78, 5.79, 5.8, 5.81, 5.82, 5.83, 5.84, 5.85, 5.86, 5.87, 5.88, 5.89, 5.9, 5.91, 5.92, 5.93, 5.94, 5.95, 5.96, 5.97, 5.98, 5.99, 6, 6.01, 6.02, 6.03, 6.04, 6.05, 6.06, 6.07, 6.08, 6.09, 6.1, 6.11, 6.12, 6.13, 6.14, 6.15, 6.16, 6.17, 6.18, 6.19, 6.2, 6.21, 6.22, 6.23, 6.24, 6.25, 6.26, 6.27, 6.28, 6.29, 6.3, 6.31, 6.32, 6.33, 6.34, 6.35, 6.36, 6.37, 6.38, 6.39, 6.4, 6.41, 6.42, 6.43, 6.44, 6.45, 6.46, 6.47, 6.48, 6.49, 6.5, 6.51, 6.52, 6.53, 6.54, 6.55, 6.56, 6.57, 6.58, 6.59, 6.6, 6.61, 6.62, 6.63, 6.64, 6.65, 6.66, 6.67, 6.68, 6.69, 6.7, 6.71, 6.72, 6.73, 6.74, 6.75, 6.76, 6.77, 6.78, 6.79, 6.8, 6.81, 6.82, 6.83, 6.84, 6.85, 6.86, 6.87, 6.88, 6.89, 6.9, 6.91, 6.92, 6.93, 6.94, 6.95, 6.96, 6.97, 6.98, 6.99, 7, 7.01, 7.02, 7.03, 7.04, 7.05, 7.06, 7.07, 7.08, 7.09, 7.1, 7.11, 7.12, 7.13, 7.14, 7.15, 7.16, 7.17, 7.18, 7.19, 7.2, 7.21, 7.22, 7.23, 7.24, 7.25, 7.26, 7.27, 7.28, 7.29, 7.3, 7.31, 7.32, 7.33, 7.34, 7.35, 7.36, 7.37, 7.38, 7.39, 7.4, 7.41, 7.42, 7.43, 7.44, 7.45, 7.46, 7.47, 7.48, 7.49, 7.5, 7.51, 7.52, 7.53, 7.54, 7.55, 7.56, 7.57, 7.58, 7.59, 7.6, 7.61, 7.62, 7.63, 7.64, 7.65, 7.66, 7.67, 7.68, 7.69, 7.7, 7.71, 7.72, 7.73, 7.74, 7.75, 7.76, 7.77, 7.78, 7.79, 7.8, 7.81, 7.82, 7.83, 7.84, 7.85, 7.86, 7.87, 7.88, 7.89, 7.9, 7.91, 7.92, 7.93, 7.94, 7.95, 7.96, 7.97, 7.98, 7.99, 8, 8.01, 8.02, 8.03, 8.04, 8.05, 8.06, 8.07, 8.08, 8.09, 8.1, 8.11, 8.12, 8.13, 8.14, 8.15, 8.16, 8.17, 8.18, 8.19, 8.2, 8.21, 8.22, 8.23, 8.24, 8.25, 8.26, 8.27, 8.28, 8.29, 8.3, 8.31, 8.32, 8.33, 8.34, 8.35, 8.36, 8.37, 8.38, 8.39, 8.4, 8.41, 8.42, 8.43, 8.44, 8.45, 8.46, 8.47, 8.48, 8.49, 8.5, 8.51, 8.52, 8.53, 8.54, 8.55, 8.56, 8.57, 8.58, 8.59, 8.6, 8.61, 8.62, 8.63, 8.64, 8.65, 8.66, 8.67, 8.68, 8.69, 8.7, 8.71, 8.72, 8.73, 8.74, 8.75, 8.76, 8.77, 8.78, 8.79, 8.8, 8.81, 8.82, 8.83, 8.84, 8.85, 8.86, 8.87, 8.88, 8.89, 8.9, 8.91, 8.92, 8.93, 8.94, 8.95, 8.96, 8.97, 8.98, 8.99, 9, 9.01, 9.02, 9.03, 9.04, 9.05, 9.06, 9.07, 9.08, 9.09, 9.1, 9.11, 9.12, 9.13, 9.14, 9.15, 9.16, 9.17, 9.18, 9.19, 9.2, 9.21, 9.22, 9.23, 9.24, 9.25, 9.26, 9.27, 9.28, 9.29, 9.3, 9.31, 9.32, 9.33, 9.34, 9.35, 9.36, 9.37, 9.38, 9.39, 9.4, 9.41, 9.42, 9.43, 9.44, 9.45, 9.46, 9.47, 9.48, 9.49, 9.5, 9.51, 9.52, 9.53, 9.54, 9.55, 9.56, 9.57, 9.58, 9.59, 9.6, 9.61, 9.62, 9.63, 9.64, 9.65, 9.66, 9.67, 9.68, 9.69, 9.7, 9.71, 9.72, 9.73, 9.74, 9.75, 9.76, 9.77, 9.78, 9.79, 9.8, 9.81, 9.82, 9.83, 9.84, 9.85, 9.86, 9.87, 9.88, 9.89, 9.9, 9.91, 9.92, 9.93, 9.94, 9.95, 9.96, 9.97, 9.98, 9.99, 10, 10.01, 10.02, 10.03, 10.04, 10.05, 10.06, 10.07, 10.08, 10.09, 10.1, 10.11, 10.12, 10.13, 10.14, 10.15, 10.16, 10.17, 10.18, 10.19, 10.2, 10.21, 10.22, 10.23, 10.24, 10.25, 10.26, 10.27, 10.28, 10.29, 10.3, 10.31, 10.32, 10.33, 10.34, 10.35, 10.36, 10.37, 10.38, 10.39, 10.4, 10.41, 10.42, 10.43, 10.44, 10.45, 10.46, 10.47, 10.48, 10.49, 10.5, 10.51, 10.52, 10.53, 10.54, 10.55, 10.56, 10.57, 10.58, 10.59, 10.6, 10.61, 10.62, 10.63, 10.64, 10.65, 10.66, 10.67, 10.68, 10.69, 10.7, 10.71, 10.72, 10.73, 10.74, 10.75, 10.76, 10.77, 10.78, 10.79, 10.8, 10.81, 10.82, 10.83, 10.84, 10.85, 10.86, 10.87, 10.88, 10.89, 10.9, 10.91, 10.92, 10.93, 10.94, 10.95, 10.96, 10.97, 10.98, 10.99, 11, 11.01, 11.02, 11.03, 11.04, 11.05, 11.06, 11.07, 11.08, 11.09, 11.1, 11.11, 11.12, 11.13, 11.14, 11.15, 11.16, 11.17, 11.18, 11.19, 11.2, 11.21, 11.22, 11.23, 11.24, 11.25, 11.26, 11.27, 11.28, 11.29, 11.3, 11.31, 11.32, 11.33, 11.34, 11.35, 11.36, 11.37, 11.38, 11.39, 11.4, 11.41, 11.42, 11.43, 11.44, 11.45, 11.46, 11.47, 11.48, 11.49, 11.5, 11.51, 11.52, 11.53, 11.54, 11.55, 11.56, 11.57, 11.58, 11.59, 11.6, 11.61, 11.62, 11.63, 11.64, 11.65, 11.66, 11.67, 11.68, 11.69, 11.7, 11.71, 11.72, 11.73, 11.74, 11.75, 11.76, 11.77, 11.78, 11.79, 11.8, 11.81, 11.82, 11.83, 11.84, 11.85, 11.86, 11.87, 11.88, 11.89, 11.9, 11.91, 11.92, 11.93, 11.94, 11.95, 11.96, 11.97, 11.98, 11.99, 12, 12.01, 12.02, 12.03, 12.04, 12.05, 12.06, 12.07, 12.08, 12.09, 12.1, 12.11, 12.12, 12.13, 12.14, 12.15, 12.16, 12.17, 12.18, 12.19, 12.2, 12.21, 12.22, 12.23, 12.24, 12.25, 12.26, 12.27, 12.28, 12.29, 12.3, 12.31, 12.32, 12.33, 12.34, 12.35, 12.36, 12.37, 12.38, 12.39, 12.4, 12.41, 12.42, 12.43, 12.44, 12.45, 12.46, 12.47, 12.48, 12.49, 12.5, 12.51, 12.52, 12.53, 12.54, 12.55, 12.56, 12.57, 12.58, 12.59, 12.6, 12.61, 12.62, 12.63, 12.64, 12.65, 12.66, 12.67, 12.68, 12.69, 12.7, 12.71, 12.72, 12.73, 12.74, 12.75, 12.76, 12.77, 12.78, 12.79, 12.8, 12.81, 12.82, 12.83, 12.84, 12.85, 12.86, 12.87, 12.88, 12.89, 12.9, 12.91, 12.92, 12.93, 12.94, 12.95, 12.96, 12.97, 12.98, 12.99, 13, 13.01, 13.02, 13.03, 13.04, 13.05, 13.06, 13.07, 13.08, 13.09, 13.1, 13.11, 13.12, 13.13, 13.14, 13.15, 13.16, 13.17, 13.18, 13.19, 13.2, 13.21, 13.22, 13.23, 13.24, 13.25, 13.26, 13.27, 13.28, 13.29, 13.3, 13.31, 13.32, 13.33, 13.34, 13.35, 13.36, 13.37, 13.38, 13.39, 13.4, 13.41, 13.42, 13.43, 13.44, 13.45, 13.46, 13.47, 13.48, 13.49, 13.5, 13.51, 13.52, 13.53, 13.54, 13.55, 13.56, 13.57, 13.58, 13.59, 13.6, 13.61, 13.62, 13.63, 13.64, 13.65, 13.66, 13.67, 13.68, 13.69, 13.7, 13.71, 13.72, 13.73, 13.74, 13.75, 13.76, 13.77, 13.78, 13.79, 13.8, 13.81, 13.82, 13.83, 13.84, 13.85, 13.86, 13.87, 13.88, 13.89, 13.9, 13.91, 13.92, 13.93, 13.94, 13.95, 13.96, 13.97, 13.98, 13.99, 14, 14.01, 14.02, 14.03, 14.04, 14.05, 14.06, 14.07, 14.08, 14.09, 14.1, 14.11, 14.12, 14.13, 14.14, 14.15, 14.16, 14.17, 14.18, 14.19, 14.2, 14.21, 14.22, 14.23, 14.24, 14.25, 14.26, 14.27, 14.28, 14.29, 14.3, 14.31, 14.32, 14.33, 14.34, 14.35, 14.36, 14.37, 14.38, 14.39, 14.4, 14.41, 14.42, 14.43, 14.44, 14.45, 14.46, 14.47, 14.48, 14.49, 14.5, 14.51, 14.52, 14.53, 14.54, 14.55, 14.56, 14.57, 14.58, 14.59, 14.6, 14.61, 14.62, 14.63, 14.64, 14.65, 14.66, 14.67, 14.68, 14.69, 14.7, 14.71, 14.72, 14.73, 14.74, 14.75, 14.76, 14.77, 14.78, 14.79, 14.8, 14.81, 14.82, 14.83, 14.84, 14.85, 14.86, 14.87, 14.88, 14.89, 14.9, 14.91, 14.92, 14.93, 14.94, 14.95, 14.96, 14.97, 14.98, 14.99, 15, 15.01, 15.02, 15.03, 15.04, 15.05, 15.06, 15.07, 15.08, 15.09, 15.1, 15.11, 15.12, 15.13, 15.14, 15.15, 15.16, 15.17, 15.18, 15.19, 15.2, 15.21, 15.22, 15.23, 15.24, 15.25, 15.26, 15.27, 15.28, 15.29, 15.3, 15.31, 15.32, 15.33, 15.34, 15.35, 15.36, 15.37, 15.38, 15.39, 15.4, 15.41, 15.42, 15.43, 15.44, 15.45, 15.46, 15.47, 15.48, 15.49, 15.5, 15.51, 15.52, 15.53, 15.54, 15.55, 15.56, 15.57, 15.58, 15.59, 15.6, 15.61, 15.62, 15.63, 15.64, 15.65, 15.66, 15.67, 15.68, 15.69, 15.7, 15.71, 15.72, 15.73, 15.74, 15.75, 15.76, 15.77, 15.78, 15.79, 15.8, 15.81, 15.82, 15.83, 15.84, 15.85, 15.86, 15.87, 15.88, 15.89, 15.9, 15.91, 15.92, 15.93, 15.94, 15.95, 15.96, 15.97, 15.98, 15.99, 16, 16.01, 16.02, 16.03, 16.04, 16.05, 16.06, 16.07, 16.08, 16.09, 16.1, 16.11, 16.12, 16.13, 16.14, 16.15, 16.16, 16.17, 16.18, 16.19, 16.2, 16.21, 16.22, 16.23, 16.24, 16.25, 16.26, 16.27, 16.28, 16.29, 16.3, 16.31, 16.32, 16.33, 16.34, 16.35, 16.36, 16.37, 16.38, 16.39, 16.4, 16.41, 16.42, 16.43, 16.44, 16.45, 16.46, 16.47, 16.48, 16.49, 16.5, 16.51, 16.52, 16.53, 16.54, 16.55, 16.56, 16.57, 16.58, 16.59, 16.6, 16.61, 16.62, 16.63, 16.64, 16.65, 16.66, 16.67, 16.68, 16.69, 16.7, 16.71, 16.72, 16.73, 16.74, 16.75, 16.76, 16.77, 16.78, 16.79, 16.8, 16.81, 16.82, 16.83, 16.84, 16.85, 16.86, 16.87, 16.88, 16.89, 16.9, 16.91, 16.92, 16.93, 16.94, 16.95, 16.96, 16.97, 16.98, 16.99, 17, 17.01, 17.02, 17.03, 17.04, 17.05, 17.06, 17.07, 17.08, 17.09, 17.1, 17.11, 17.12, 17.13, 17.14, 17.15, 17.16, 17.17, 17.18, 17.19, 17.2, 17.21, 17.22, 17.23, 17.24, 17.25, 17.26, 17.27, 17.28, 17.29, 17.3, 17.31, 17.32, 17.33, 17.34, 17.35, 17.36, 17.37, 17.38, 17.39, 17.4, 17.41, 17.42, 17.43, 17.44, 17.45, 17.46, 17.47, 17.48, 17.49, 17.5, 17.51, 17.52, 17.53, 17.54, 17.55, 17.56, 17.57, 17.58, 17.59, 17.6, 17.61, 17.62, 17.63, 17.64, 17.65, 17.66, 17.67, 17.68, 17.69, 17.7, 17.71, 17.72, 17.73, 17.74, 17.75, 17.76, 17.77, 17.78, 17.79, 17.8, 17.81, 17.82, 17.83, 17.84, 17.85, 17.86, 17.87, 17.88, 17.89, 17.9, 17.91, 17.92, 17.93, 17.94, 17.95, 17.96, 17.97, 17.98, 17.99, 18, 18.01, 18.02, 18.03, 18.04, 18.05, 18.06, 18.07, 18.08, 18.09, 18.1, 18.11, 18.12, 18.13, 18.14, 18.15, 18.16, 18.17, 18.18, 18.19, 18.2, 18.21, 18.22, 18.23, 18.24, 18.25, 18.26, 18.27, 18.28, 18.29, 18.3, 18.31, 18.32, 18.33, 18.34, 18.35, 18.36, 18.37, 18.38, 18.39, 18.4, 18.41, 18.42, 18.43, 18.44, 18.45, 18.46, 18.47, 18.48, 18.49, 18.5, 18.51, 18.52, 18.53, 18.54, 18.55, 18.56, 18.57, 18.58, 18.59, 18.6, 18.61, 18.62, 18.63, 18.64, 18.65, 18.66, 18.67, 18.68, 18.69, 18.7, 18.71, 18.72, 18.73, 18.74, 18.75, 18.76, 18.77, 18.78, 18.79, 18.8, 18.81, 18.82, 18.83, 18.84, 18.85, 18.86, 18.87, 18.88, 18.89, 18.9, 18.91, 18.92, 18.93, 18.94, 18.95, 18.96, 18.97, 18.98, 18.99, 19, 19.01, 19.02, 19.03, 19.04, 19.05, 19.06, 19.07, 19.08, 19.09, 19.1, 19.11, 19.12, 19.13, 19.14, 19.15, 19.16, 19.17, 19.18, 19.19, 19.2, 19.21, 19.22, 19.23, 19.24, 19.25, 19.26, 19.27, 19.28, 19.29, 19.3, 19.31, 19.32, 19.33, 19.34, 19.35, 19.36, 19.37, 19.38, 19.39, 19.4, 19.41, 19.42, 19.43, 19.44, 19.45, 19.46, 19.47, 19.48, 19.49, 19.5, 19.51, 19.52, 19.53, 19.54, 19.55, 19.56, 19.57, 19.58, 19.59, 19.6, 19.61, 19.62, 19.63, 19.64, 19.65, 19.66, 19.67, 19.68, 19.69, 19.7, 19.71, 19.72, 19.73, 19.74, 19.75, 19.76, 19.77, 19.78, 19.79, 19.8, 19.81, 19.82, 19.83, 19.84, 19.85, 19.86, 19.87, 19.88, 19.89, 19.9, 19.91, 19.92, 19.93, 19.94, 19.95, 19.96, 19.97, 19.98, or 19.99 mg/day, or any amount in between, above, or below.

The tamoxifen or derivative, formulation, or metabolite thereof may be given at the same dose given for cancer. For example, the dose can be 20, 20.01, 20.02, 20.03, 20.04, 20.05, 20.06, 20.07, 20.08, 20.09, 20.1, 20.11, 20.12, 20.13, 20.14, 20.15, 20.16, 20.17, 20.18, 20.19, 20.2, 20.21, 20.22, 20.23, 20.24, 20.25, 20.26, 20.27, 20.28, 20.29, 20.3, 20.31, 20.32, 20.33, 20.34, 20.35, 20.36, 20.37, 20.38, 20.39, 20.4, 20.41, 20.42, 20.43, 20.44, 20.45, 20.46, 20.47, 20.48, 20.49, 20.5, 20.51, 20.52, 20.53, 20.54, 20.55, 20.56, 20.57, 20.58, 20.59, 20.6, 20.61, 20.62, 20.63, 20.64, 20.65, 20.66, 20.67, 20.68, 20.69, 20.7, 20.71, 20.72, 20.73, 20.74, 20.75, 20.76, 20.77, 20.78, 20.79, 20.8, 20.81, 20.82, 20.83, 20.84, 20.85, 20.86, 20.87, 20.88, 20.89, 20.9, 20.91, 20.92, 20.93, 20.94, 20.95, 20.96, 20.97, 20.98, 20.99, 21, 21.01, 21.02, 21.03, 21.04, 21.05, 21.06, 21.07, 21.08, 21.09, 21.1, 21.11, 21.12, 21.13, 21.14, 21.15, 21.16, 21.17, 21.18, 21.19, 21.2, 21.21, 21.22, 21.23, 21.24, 21.25, 21.26, 21.27, 21.28, 21.29, 21.3, 21.31, 21.32, 21.33, 21.34, 21.35, 21.36, 21.37, 21.38, 21.39, 21.4, 21.41, 21.42, 21.43, 21.44, 21.45, 21.46, 21.47, 21.48, 21.49, 21.5, 21.51, 21.52, 21.53, 21.54, 21.55, 21.56, 21.57, 21.58, 21.59, 21.6, 21.61, 21.62, 21.63, 21.64, 21.65, 21.66, 21.67, 21.68, 21.69, 21.7, 21.71, 21.72, 21.73, 21.74, 21.75, 21.76, 21.77, 21.78, 21.79, 21.8, 21.81, 21.82, 21.83, 21.84, 21.85, 21.86, 21.87, 21.88, 21.89, 21.9, 21.91, 21.92, 21.93, 21.94, 21.95, 21.96, 21.97, 21.98, 21.99, 22, 22.01, 22.02, 22.03, 22.04, 22.05, 22.06, 22.07, 22.08, 22.09, 22.1, 22.11, 22.12, 22.13, 22.14, 22.15, 22.16, 22.17, 22.18, 22.19, 22.2, 22.21, 22.22, 22.23, 22.24, 22.25, 22.26, 22.27, 22.28, 22.29, 22.3, 22.31, 22.32, 22.33, 22.34, 22.35, 22.36, 22.37, 22.38, 22.39, 22.4, 22.41, 22.42, 22.43, 22.44, 22.45, 22.46, 22.47, 22.48, 22.49, 22.5, 22.51, 22.52, 22.53, 22.54, 22.55, 22.56, 22.57, 22.58, 22.59, 22.6, 22.61, 22.62, 22.63, 22.64, 22.65, 22.66, 22.67, 22.68, 22.69, 22.7, 22.71, 22.72, 22.73, 22.74, 22.75, 22.76, 22.77, 22.78, 22.79, 22.8, 22.81, 22.82, 22.83, 22.84, 22.85, 22.86, 22.87, 22.88, 22.89, 22.9, 22.91, 22.92, 22.93, 22.94, 22.95, 22.96, 22.97, 22.98, 22.99, 23, 23.01, 23.02, 23.03, 23.04, 23.05, 23.06, 23.07, 23.08, 23.09, 23.1, 23.11, 23.12, 23.13, 23.14, 23.15, 23.16, 23.17, 23.18, 23.19, 23.2, 23.21, 23.22, 23.23, 23.24, 23.25, 23.26, 23.27, 23.28, 23.29, 23.3, 23.31, 23.32, 23.33, 23.34, 23.35, 23.36, 23.37, 23.38, 23.39, 23.4, 23.41, 23.42, 23.43, 23.44, 23.45, 23.46, 23.47, 23.48, 23.49, 23.5, 23.51, 23.52, 23.53, 23.54, 23.55, 23.56, 23.57, 23.58, 23.59, 23.6, 23.61, 23.62, 23.63, 23.64, 23.65, 23.66, 23.67, 23.68, 23.69, 23.7, 23.71, 23.72, 23.73, 23.74, 23.75, 23.76, 23.77, 23.78, 23.79, 23.8, 23.81, 23.82, 23.83, 23.84, 23.85, 23.86, 23.87, 23.88, 23.89, 23.9, 23.91, 23.92, 23.93, 23.94, 23.95, 23.96, 23.97, 23.98, 23.99, 24, 24.01, 24.02, 24.03, 24.04, 24.05, 24.06, 24.07, 24.08, 24.09, 24.1, 24.11, 24.12, 24.13, 24.14, 24.15, 24.16, 24.17, 24.18, 24.19, 24.2, 24.21, 24.22, 24.23, 24.24, 24.25, 24.26, 24.27, 24.28, 24.29, 24.3, 24.31, 24.32, 24.33, 24.34, 24.35, 24.36, 24.37, 24.38, 24.39, 24.4, 24.41, 24.42, 24.43, 24.44, 24.45, 24.46, 24.47, 24.48, 24.49, 24.5, 24.51, 24.52, 24.53, 24.54, 24.55, 24.56, 24.57, 24.58, 24.59, 24.6, 24.61, 24.62, 24.63, 24.64, 24.65, 24.66, 24.67, 24.68, 24.69, 24.7, 24.71, 24.72, 24.73, 24.74, 24.75, 24.76, 24.77, 24.78, 24.79, 24.8, 24.81, 24.82, 24.83, 24.84, 24.85, 24.86, 24.87, 24.88, 24.89, 24.9, 24.91, 24.92, 24.93, 24.94, 24.95, 24.96, 24.97, 24.98, 24.99, 25, 25.01, 25.02, 25.03, 25.04, 25.05, 25.06, 25.07, 25.08, 25.09, 25.1, 25.11, 25.12, 25.13, 25.14, 25.15, 25.16, 25.17, 25.18, 25.19, 25.2, 25.21, 25.22, 25.23, 25.24, 25.25, 25.26, 25.27, 25.28, 25.29, 25.3, 25.31, 25.32, 25.33, 25.34, 25.35, 25.36, 25.37, 25.38, 25.39, 25.4, 25.41, 25.42, 25.43, 25.44, 25.45, 25.46, 25.47, 25.48, 25.49, 25.5, 25.51, 25.52, 25.53, 25.54, 25.55, 25.56, 25.57, 25.58, 25.59, 25.6, 25.61, 25.62, 25.63, 25.64, 25.65, 25.66, 25.67, 25.68, 25.69, 25.7, 25.71, 25.72, 25.73, 25.74, 25.75, 25.76, 25.77, 25.78, 25.79, 25.8, 25.81, 25.82, 25.83, 25.84, 25.85, 25.86, 25.87, 25.88, 25.89, 25.9, 25.91, 25.92, 25.93, 25.94, 25.95, 25.96, 25.97, 25.98, 25.99, 26, 26.01, 26.02, 26.03, 26.04, 26.05, 26.06, 26.07, 26.08, 26.09, 26.1, 26.11, 26.12, 26.13, 26.14, 26.15, 26.16, 26.17, 26.18, 26.19, 26.2, 26.21, 26.22, 26.23, 26.24, 26.25, 26.26, 26.27, 26.28, 26.29, 26.3, 26.31, 26.32, 26.33, 26.34, 26.35, 26.36, 26.37, 26.38, 26.39, 26.4, 26.41, 26.42, 26.43, 26.44, 26.45, 26.46, 26.47, 26.48, 26.49, 26.5, 26.51, 26.52, 26.53, 26.54, 26.55, 26.56, 26.57, 26.58, 26.59, 26.6, 26.61, 26.62, 26.63, 26.64, 26.65, 26.66, 26.67, 26.68, 26.69, 26.7, 26.71, 26.72, 26.73, 26.74, 26.75, 26.76, 26.77, 26.78, 26.79, 26.8, 26.81, 26.82, 26.83, 26.84, 26.85, 26.86, 26.87, 26.88, 26.89, 26.9, 26.91, 26.92, 26.93, 26.94, 26.95, 26.96, 26.97, 26.98, 26.99, 27, 27.01, 27.02, 27.03, 27.04, 27.05, 27.06, 27.07, 27.08, 27.09, 27.1, 27.11, 27.12, 27.13, 27.14, 27.15, 27.16, 27.17, 27.18, 27.19, 27.2, 27.21, 27.22, 27.23, 27.24, 27.25, 27.26, 27.27, 27.28, 27.29, 27.3, 27.31, 27.32, 27.33, 27.34, 27.35, 27.36, 27.37, 27.38, 27.39, 27.4, 27.41, 27.42, 27.43, 27.44, 27.45, 27.46, 27.47, 27.48, 27.49, 27.5, 27.51, 27.52, 27.53, 27.54, 27.55, 27.56, 27.57, 27.58, 27.59, 27.6, 27.61, 27.62, 27.63, 27.64, 27.65, 27.66, 27.67, 27.68, 27.69, 27.7, 27.71, 27.72, 27.73, 27.74, 27.75, 27.76, 27.77, 27.78, 27.79, 27.8, 27.81, 27.82, 27.83, 27.84, 27.85, 27.86, 27.87, 27.88, 27.89, 27.9, 27.91, 27.92, 27.93, 27.94, 27.95, 27.96, 27.97, 27.98, 27.99, 28, 28.01, 28.02, 28.03, 28.04, 28.05, 28.06, 28.07, 28.08, 28.09, 28.1, 28.11, 28.12, 28.13, 28.14, 28.15, 28.16, 28.17, 28.18, 28.19, 28.2, 28.21, 28.22, 28.23, 28.24, 28.25, 28.26, 28.27, 28.28, 28.29, 28.3, 28.31, 28.32, 28.33, 28.34, 28.35, 28.36, 28.37, 28.38, 28.39, 28.4, 28.41, 28.42, 28.43, 28.44, 28.45, 28.46, 28.47, 28.48, 28.49, 28.5, 28.51, 28.52, 28.53, 28.54, 28.55, 28.56, 28.57, 28.58, 28.59, 28.6, 28.61, 28.62, 28.63, 28.64, 28.65, 28.66, 28.67, 28.68, 28.69, 28.7, 28.71, 28.72, 28.73, 28.74, 28.75, 28.76, 28.77, 28.78, 28.79, 28.8, 28.81, 28.82, 28.83, 28.84, 28.85, 28.86, 28.87, 28.88, 28.89, 28.9, 28.91, 28.92, 28.93, 28.94, 28.95, 28.96, 28.97, 28.98, 28.99, 29, 29.01, 29.02, 29.03, 29.04, 29.05, 29.06, 29.07, 29.08, 29.09, 29.1, 29.11, 29.12, 29.13, 29.14, 29.15, 29.16, 29.17, 29.18, 29.19, 29.2, 29.21, 29.22, 29.23, 29.24, 29.25, 29.26, 29.27, 29.28, 29.29, 29.3, 29.31, 29.32, 29.33, 29.34, 29.35, 29.36, 29.37, 29.38, 29.39, 29.4, 29.41, 29.42, 29.43, 29.44, 29.45, 29.46, 29.47, 29.48, 29.49, 29.5, 29.51, 29.52, 29.53, 29.54, 29.55, 29.56, 29.57, 29.58, 29.59, 29.6, 29.61, 29.62, 29.63, 29.64, 29.65, 29.66, 29.67, 29.68, 29.69, 29.7, 29.71, 29.72, 29.73, 29.74, 29.75, 29.76, 29.77, 29.78, 29.79, 29.8, 29.81, 29.82, 29.83, 29.84, 29.85, 29.86, 29.87, 29.88, 29.89, 29.9, 29.91, 29.92, 29.93, 29.94, 29.95, 29.96, 29.97, 29.98, 29.99, 30, 30.01, 30.02, 30.03, 30.04, 30.05, 30.06, 30.07, 30.08, 30.09, 30.1, 30.11, 30.12, 30.13, 30.14, 30.15, 30.16, 30.17, 30.18, 30.19, 30.2, 30.21, 30.22, 30.23, 30.24, 30.25, 30.26, 30.27, 30.28, 30.29, 30.3, 30.31, 30.32, 30.33, 30.34, 30.35, 30.36, 30.37, 30.38, 30.39, 30.4, 30.41, 30.42, 30.43, 30.44, 30.45, 30.46, 30.47, 30.48, 30.49, 30.5, 30.51, 30.52, 30.53, 30.54, 30.55, 30.56, 30.57, 30.58, 30.59, 30.6, 30.61, 30.62, 30.63, 30.64, 30.65, 30.66, 30.67, 30.68, 30.69, 30.7, 30.71, 30.72, 30.73, 30.74, 30.75, 30.76, 30.77, 30.78, 30.79, 30.8, 30.81, 30.82, 30.83, 30.84, 30.85, 30.86, 30.87, 30.88, 30.89, 30.9, 30.91, 30.92, 30.93, 30.94, 30.95, 30.96, 30.97, 30.98, 30.99, 31, 31.01, 31.02, 31.03, 31.04, 31.05, 31.06, 31.07, 31.08, 31.09, 31.1, 31.11, 31.12, 31.13, 31.14, 31.15, 31.16, 31.17, 31.18, 31.19, 31.2, 31.21, 31.22, 31.23, 31.24, 31.25, 31.26, 31.27, 31.28, 31.29, 31.3, 31.31, 31.32, 31.33, 31.34, 31.35, 31.36, 31.37, 31.38, 31.39, 31.4, 31.41, 31.42, 31.43, 31.44, 31.45, 31.46, 31.47, 31.48, 31.49, 31.5, 31.51, 31.52, 31.53, 31.54, 31.55, 31.56, 31.57, 31.58, 31.59, 31.6, 31.61, 31.62, 31.63, 31.64, 31.65, 31.66, 31.67, 31.68, 31.69, 31.7, 31.71, 31.72, 31.73, 31.74, 31.75, 31.76, 31.77, 31.78, 31.79, 31.8, 31.81, 31.82, 31.83, 31.84, 31.85, 31.86, 31.87, 31.88, 31.89, 31.9, 31.91, 31.92, 31.93, 31.94, 31.95, 31.96, 31.97, 31.98, 31.99, 32, 32.01, 32.02, 32.03, 32.04, 32.05, 32.06, 32.07, 32.08, 32.09, 32.1, 32.11, 32.12, 32.13, 32.14, 32.15, 32.16, 32.17, 32.18, 32.19, 32.2, 32.21, 32.22, 32.23, 32.24, 32.25, 32.26, 32.27, 32.28, 32.29, 32.3, 32.31, 32.32, 32.33, 32.34, 32.35, 32.36, 32.37, 32.38, 32.39, 32.4, 32.41, 32.42, 32.43, 32.44, 32.45, 32.46, 32.47, 32.48, 32.49, 32.5, 32.51, 32.52, 32.53, 32.54, 32.55, 32.56, 32.57, 32.58, 32.59, 32.6, 32.61, 32.62, 32.63, 32.64, 32.65, 32.66, 32.67, 32.68, 32.69, 32.7, 32.71, 32.72, 32.73, 32.74, 32.75, 32.76, 32.77, 32.78, 32.79, 32.8, 32.81, 32.82, 32.83, 32.84, 32.85, 32.86, 32.87, 32.88, 32.89, 32.9, 32.91, 32.92, 32.93, 32.94, 32.95, 32.96, 32.97, 32.98, 32.99, 33, 33.01, 33.02, 33.03, 33.04, 33.05, 33.06, 33.07, 33.08, 33.09, 33.1, 33.11, 33.12, 33.13, 33.14, 33.15, 33.16, 33.17, 33.18, 33.19, 33.2, 33.21, 33.22, 33.23, 33.24, 33.25, 33.26, 33.27, 33.28, 33.29, 33.3, 33.31, 33.32, 33.33, 33.34, 33.35, 33.36, 33.37, 33.38, 33.39, 33.4, 33.41, 33.42, 33.43, 33.44, 33.45, 33.46, 33.47, 33.48, 33.49, 33.5, 33.51, 33.52, 33.53, 33.54, 33.55, 33.56, 33.57, 33.58, 33.59, 33.6, 33.61, 33.62, 33.63, 33.64, 33.65, 33.66, 33.67, 33.68, 33.69, 33.7, 33.71, 33.72, 33.73, 33.74, 33.75, 33.76, 33.77, 33.78, 33.79, 33.8, 33.81, 33.82, 33.83, 33.84, 33.85, 33.86, 33.87, 33.88, 33.89, 33.9, 33.91, 33.92, 33.93, 33.94, 33.95, 33.96, 33.97, 33.98, 33.99, 34, 34.01, 34.02, 34.03, 34.04, 34.05, 34.06, 34.07, 34.08, 34.09, 34.1, 34.11, 34.12, 34.13, 34.14, 34.15, 34.16, 34.17, 34.18, 34.19, 34.2, 34.21, 34.22, 34.23, 34.24, 34.25, 34.26, 34.27, 34.28, 34.29, 34.3, 34.31, 34.32, 34.33, 34.34, 34.35, 34.36, 34.37, 34.38, 34.39, 34.4, 34.41, 34.42, 34.43, 34.44, 34.45, 34.46, 34.47, 34.48, 34.49, 34.5, 34.51, 34.52, 34.53, 34.54, 34.55, 34.56, 34.57, 34.58, 34.59, 34.6, 34.61, 34.62, 34.63, 34.64, 34.65, 34.66, 34.67, 34.68, 34.69, 34.7, 34.71, 34.72, 34.73, 34.74, 34.75, 34.76, 34.77, 34.78, 34.79, 34.8, 34.81, 34.82, 34.83, 34.84, 34.85, 34.86, 34.87, 34.88, 34.89, 34.9, 34.91, 34.92, 34.93, 34.94, 34.95, 34.96, 34.97, 34.98, 34.99, 35, 35.01, 35.02, 35.03, 35.04, 35.05, 35.06, 35.07, 35.08, 35.09, 35.1, 35.11, 35.12, 35.13, 35.14, 35.15, 35.16, 35.17, 35.18, 35.19, 35.2, 35.21, 35.22, 35.23, 35.24, 35.25, 35.26, 35.27, 35.28, 35.29, 35.3, 35.31, 35.32, 35.33, 35.34, 35.35, 35.36, 35.37, 35.38, 35.39, 35.4, 35.41, 35.42, 35.43, 35.44, 35.45, 35.46, 35.47, 35.48, 35.49, 35.5, 35.51, 35.52, 35.53, 35.54, 35.55, 35.56, 35.57, 35.58, 35.59, 35.6, 35.61, 35.62, 35.63, 35.64, 35.65, 35.66, 35.67, 35.68, 35.69, 35.7, 35.71, 35.72, 35.73, 35.74, 35.75, 35.76, 35.77, 35.78, 35.79, 35.8, 35.81, 35.82, 35.83, 35.84, 35.85, 35.86, 35.87, 35.88, 35.89, 35.9, 35.91, 35.92, 35.93, 35.94, 35.95, 35.96, 35.97, 35.98, 35.99, 36, 36.01, 36.02, 36.03, 36.04, 36.05, 36.06, 36.07, 36.08, 36.09, 36.1, 36.11, 36.12, 36.13, 36.14, 36.15, 36.16, 36.17, 36.18, 36.19, 36.2, 36.21, 36.22, 36.23, 36.24, 36.25, 36.26, 36.27, 36.28, 36.29, 36.3, 36.31, 36.32, 36.33, 36.34, 36.35, 36.36, 36.37, 36.38, 36.39, 36.4, 36.41, 36.42, 36.43, 36.44, 36.45, 36.46, 36.47, 36.48, 36.49, 36.5, 36.51, 36.52, 36.53, 36.54, 36.55, 36.56, 36.57, 36.58, 36.59, 36.6, 36.61, 36.62, 36.63, 36.64, 36.65, 36.66, 36.67, 36.68, 36.69, 36.7, 36.71, 36.72, 36.73, 36.74, 36.75, 36.76, 36.77, 36.78, 36.79, 36.8, 36.81, 36.82, 36.83, 36.84, 36.85, 36.86, 36.87, 36.88, 36.89, 36.9, 36.91, 36.92, 36.93, 36.94, 36.95, 36.96, 36.97, 36.98, 36.99, 37, 37.01, 37.02, 37.03, 37.04, 37.05, 37.06, 37.07, 37.08, 37.09, 37.1, 37.11, 37.12, 37.13, 37.14, 37.15, 37.16, 37.17, 37.18, 37.19, 37.2, 37.21, 37.22, 37.23, 37.24, 37.25, 37.26, 37.27, 37.28, 37.29, 37.3, 37.31, 37.32, 37.33, 37.34, 37.35, 37.36, 37.37, 37.38, 37.39, 37.4, 37.41, 37.42, 37.43, 37.44, 37.45, 37.46, 37.47, 37.48, 37.49, 37.5, 37.51, 37.52, 37.53, 37.54, 37.55, 37.56, 37.57, 37.58, 37.59, 37.6, 37.61, 37.62, 37.63, 37.64, 37.65, 37.66, 37.67, 37.68, 37.69, 37.7, 37.71, 37.72, 37.73, 37.74, 37.75, 37.76, 37.77, 37.78, 37.79, 37.8, 37.81, 37.82, 37.83, 37.84, 37.85, 37.86, 37.87, 37.88, 37.89, 37.9, 37.91, 37.92, 37.93, 37.94, 37.95, 37.96, 37.97, 37.98, 37.99, 38, 38.01, 38.02, 38.03, 38.04, 38.05, 38.06, 38.07, 38.08, 38.09, 38.1, 38.11, 38.12, 38.13, 38.14, 38.15, 38.16, 38.17, 38.18, 38.19, 38.2, 38.21, 38.22, 38.23, 38.24, 38.25, 38.26, 38.27, 38.28, 38.29, 38.3, 38.31, 38.32, 38.33, 38.34, 38.35, 38.36, 38.37, 38.38, 38.39, 38.4, 38.41, 38.42, 38.43, 38.44, 38.45, 38.46, 38.47, 38.48, 38.49, 38.5, 38.51, 38.52, 38.53, 38.54, 38.55, 38.56, 38.57, 38.58, 38.59, 38.6, 38.61, 38.62, 38.63, 38.64, 38.65, 38.66, 38.67, 38.68, 38.69, 38.7, 38.71, 38.72, 38.73, 38.74, 38.75, 38.76, 38.77, 38.78, 38.79, 38.8, 38.81, 38.82, 38.83, 38.84, 38.85, 38.86, 38.87, 38.88, 38.89, 38.9, 38.91, 38.92, 38.93, 38.94, 38.95, 38.96, 38.97, 38.98, 38.99, 39, 39.01, 39.02, 39.03, 39.04, 39.05, 39.06, 39.07, 39.08, 39.09, 39.1, 39.11, 39.12, 39.13, 39.14, 39.15, 39.16, 39.17, 39.18, 39.19, 39.2, 39.21, 39.22, 39.23, 39.24, 39.25, 39.26, 39.27, 39.28, 39.29, 39.3, 39.31, 39.32, 39.33, 39.34, 39.35, 39.36, 39.37, 39.38, 39.39, 39.4, 39.41, 39.42, 39.43, 39.44, 39.45, 39.46, 39.47, 39.48, 39.49, 39.5, 39.51, 39.52, 39.53, 39.54, 39.55, 39.56, 39.57, 39.58, 39.59, 39.6, 39.61, 39.62, 39.63, 39.64, 39.65, 39.66, 39.67, 39.68, 39.69, 39.7, 39.71, 39.72, 39.73, 39.74, 39.75, 39.76, 39.77, 39.78, 39.79, 39.8, 39.81, 39.82, 39.83, 39.84, 39.85, 39.86, 39.87, 39.88, 39.89, 39.9, 39.91, 39.92, 39.93, 39.94, 39.95, 39.96, 39.97, 39.98, 39.99, or 40 mg/day, or any amount lower, higher, or in between.

The tamoxifen, or a derivative, formulation, or metabolite thereof can also be given in an amount greater than an amount to treat or prevent cancer. For example, it can be given at a dose of 40.01, 40.02, 40.03, 40.04, 40.05, 40.06, 40.07, 40.08, 40.09, 40.1, 40.11, 40.12, 40.13, 40.14, 40.15, 40.16, 40.17, 40.18, 40.19, 40.2, 40.21, 40.22, 40.23, 40.24, 40.25, 40.26, 40.27, 40.28, 40.29, 40.3, 40.31, 40.32, 40.33, 40.34, 40.35, 40.36, 40.37, 40.38, 40.39, 40.4, 40.41, 40.42, 40.43, 40.44, 40.45, 40.46, 40.47, 40.48, 40.49, 40.5, 40.51, 40.52, 40.53, 40.54, 40.55, 40.56, 40.57, 40.58, 40.59, 40.6, 40.61, 40.62, 40.63, 40.64, 40.65, 40.66, 40.67, 40.68, 40.69, 40.7, 40.71, 40.72, 40.73, 40.74, 40.75, 40.76, 40.77, 40.78, 40.79, 40.8, 40.81, 40.82, 40.83, 40.84, 40.85, 40.86, 40.87, 40.88, 40.89, 40.9, 40.91, 40.92, 40.93, 40.94, 40.95, 40.96, 40.97, 40.98, 40.99, 41, 41.01, 41.02, 41.03, 41.04, 41.05, 41.06, 41.07, 41.08, 41.09, 41.1, 41.11, 41.12, 41.13, 41.14, 41.15, 41.16, 41.17, 41.18, 41.19, 41.2, 41.21, 41.22, 41.23, 41.24, 41.25, 41.26, 41.27, 41.28, 41.29, 41.3, 41.31, 41.32, 41.33, 41.34, 41.35, 41.36, 41.37, 41.38, 41.39, 41.4, 41.41, 41.42, 41.43, 41.44, 41.45, 41.46, 41.47, 41.48, 41.49, 41.5, 41.51, 41.52, 41.53, 41.54, 41.55, 41.56, 41.57, 41.58, 41.59, 41.6, 41.61, 41.62, 41.63, 41.64, 41.65, 41.66, 41.67, 41.68, 41.69, 41.7, 41.71, 41.72, 41.73, 41.74, 41.75, 41.76, 41.77, 41.78, 41.79, 41.8, 41.81, 41.82, 41.83, 41.84, 41.85, 41.86, 41.87, 41.88, 41.89, 41.9, 41.91, 41.92, 41.93, 41.94, 41.95, 41.96, 41.97, 41.98, 41.99, 42, 42.01, 42.02, 42.03, 42.04, 42.05, 42.06, 42.07, 42.08, 42.09, 42.1, 42.11, 42.12, 42.13, 42.14, 42.15, 42.16, 42.17, 42.18, 42.19, 42.2, 42.21, 42.22, 42.23, 42.24, 42.25, 42.26, 42.27, 42.28, 42.29, 42.3, 42.31, 42.32, 42.33, 42.34, 42.35, 42.36, 42.37, 42.38, 42.39, 42.4, 42.41, 42.42, 42.43, 42.44, 42.45, 42.46, 42.47, 42.48, 42.49, 42.5, 42.51, 42.52, 42.53, 42.54, 42.55, 42.56, 42.57, 42.58, 42.59, 42.6, 42.61, 42.62, 42.63, 42.64, 42.65, 42.66, 42.67, 42.68, 42.69, 42.7, 42.71, 42.72, 42.73, 42.74, 42.75, 42.76, 42.77, 42.78, 42.79, 42.8, 42.81, 42.82, 42.83, 42.84, 42.85, 42.86, 42.87, 42.88, 42.89, 42.9, 42.91, 42.92, 42.93, 42.94, 42.95, 42.96, 42.97, 42.98, 42.99, 43, 43.01, 43.02, 43.03, 43.04, 43.05, 43.06, 43.07, 43.08, 43.09, 43.1, 43.11, 43.12, 43.13, 43.14, 43.15, 43.16, 43.17, 43.18, 43.19, 43.2, 43.21, 43.22, 43.23, 43.24, 43.25, 43.26, 43.27, 43.28, 43.29, 43.3, 43.31, 43.32, 43.33, 43.34, 43.35, 43.36, 43.37, 43.38, 43.39, 43.4, 43.41, 43.42, 43.43, 43.44, 43.45, 43.46, 43.47, 43.48, 43.49, 43.5, 43.51, 43.52, 43.53, 43.54, 43.55, 43.56, 43.57, 43.58, 43.59, 43.6, 43.61, 43.62, 43.63, 43.64, 43.65, 43.66, 43.67, 43.68, 43.69, 43.7, 43.71, 43.72, 43.73, 43.74, 43.75, 43.76, 43.77, 43.78, 43.79, 43.8, 43.81, 43.82, 43.83, 43.84, 43.85, 43.86, 43.87, 43.88, 43.89, 43.9, 43.91, 43.92, 43.93, 43.94, 43.95, 43.96, 43.97, 43.98, 43.99, 44, 44.01, 44.02, 44.03, 44.04, 44.05, 44.06, 44.07, 44.08, 44.09, 44.1, 44.11, 44.12, 44.13, 44.14, 44.15, 44.16, 44.17, 44.18, 44.19, 44.2, 44.21, 44.22, 44.23, 44.24, 44.25, 44.26, 44.27, 44.28, 44.29, 44.3, 44.31, 44.32, 44.33, 44.34, 44.35, 44.36, 44.37, 44.38, 44.39, 44.4, 44.41, 44.42, 44.43, 44.44, 44.45, 44.46, 44.47, 44.48, 44.49, 44.5, 44.51, 44.52, 44.53, 44.54, 44.55, 44.56, 44.57, 44.58, 44.59, 44.6, 44.61, 44.62, 44.63, 44.64, 44.65, 44.66, 44.67, 44.68, 44.69, 44.7, 44.71, 44.72, 44.73, 44.74, 44.75, 44.76, 44.77, 44.78, 44.79, 44.8, 44.81, 44.82, 44.83, 44.84, 44.85, 44.86, 44.87, 44.88, 44.89, 44.9, 44.91, 44.92, 44.93, 44.94, 44.95, 44.96, 44.97, 44.98, 44.99, 45, 45.01, 45.02, 45.03, 45.04, 45.05, 45.06, 45.07, 45.08, 45.09, 45.1, 45.11, 45.12, 45.13, 45.14, 45.15, 45.16, 45.17, 45.18, 45.19, 45.2, 45.21, 45.22, 45.23, 45.24, 45.25, 45.26, 45.27, 45.28, 45.29, 45.3, 45.31, 45.32, 45.33, 45.34, 45.35, 45.36, 45.37, 45.38, 45.39, 45.4, 45.41, 45.42, 45.43, 45.44, 45.45, 45.46, 45.47, 45.48, 45.49, 45.5, 45.51, 45.52, 45.53, 45.54, 45.55, 45.56, 45.57, 45.58, 45.59, 45.6, 45.61, 45.62, 45.63, 45.64, 45.65, 45.66, 45.67, 45.68, 45.69, 45.7, 45.71, 45.72, 45.73, 45.74, 45.75, 45.76, 45.77, 45.78, 45.79, 45.8, 45.81, 45.82, 45.83, 45.84, 45.85, 45.86, 45.87, 45.88, 45.89, 45.9, 45.91, 45.92, 45.93, 45.94, 45.95, 45.96, 45.97, 45.98, 45.99, 46, 46.01, 46.02, 46.03, 46.04, 46.05, 46.06, 46.07, 46.08, 46.09, 46.1, 46.11, 46.12, 46.13, 46.14, 46.15, 46.16, 46.17, 46.18, 46.19, 46.2, 46.21, 46.22, 46.23, 46.24, 46.25, 46.26, 46.27, 46.28, 46.29, 46.3, 46.31, 46.32, 46.33, 46.34, 46.35, 46.36, 46.37, 46.38, 46.39, 46.4, 46.41, 46.42, 46.43, 46.44, 46.45, 46.46, 46.47, 46.48, 46.49, 46.5, 46.51, 46.52, 46.53, 46.54, 46.55, 46.56, 46.57, 46.58, 46.59, 46.6, 46.61, 46.62, 46.63, 46.64, 46.65, 46.66, 46.67, 46.68, 46.69, 46.7, 46.71, 46.72, 46.73, 46.74, 46.75, 46.76, 46.77, 46.78, 46.79, 46.8, 46.81, 46.82, 46.83, 46.84, 46.85, 46.86, 46.87, 46.88, 46.89, 46.9, 46.91, 46.92, 46.93, 46.94, 46.95, 46.96, 46.97, 46.98, 46.99, 47, 47.01, 47.02, 47.03, 47.04, 47.05, 47.06, 47.07, 47.08, 47.09, 47.1, 47.11, 47.12, 47.13, 47.14, 47.15, 47.16, 47.17, 47.18, 47.19, 47.2, 47.21, 47.22, 47.23, 47.24, 47.25, 47.26, 47.27, 47.28, 47.29, 47.3, 47.31, 47.32, 47.33, 47.34, 47.35, 47.36, 47.37, 47.38, 47.39, 47.4, 47.41, 47.42, 47.43, 47.44, 47.45, 47.46, 47.47, 47.48, 47.49, 47.5, 47.51, 47.52, 47.53, 47.54, 47.55, 47.56, 47.57, 47.58, 47.59, 47.6, 47.61, 47.62, 47.63, 47.64, 47.65, 47.66, 47.67, 47.68, 47.69, 47.7, 47.71, 47.72, 47.73, 47.74, 47.75, 47.76, 47.77, 47.78, 47.79, 47.8, 47.81, 47.82, 47.83, 47.84, 47.85, 47.86, 47.87, 47.88, 47.89, 47.9, 47.91, 47.92, 47.93, 47.94, 47.95, 47.96, 47.97, 47.98, 47.99, 48, 48.01, 48.02, 48.03, 48.04, 48.05, 48.06, 48.07, 48.08, 48.09, 48.1, 48.11, 48.12, 48.13, 48.14, 48.15, 48.16, 48.17, 48.18, 48.19, 48.2, 48.21, 48.22, 48.23, 48.24, 48.25, 48.26, 48.27, 48.28, 48.29, 48.3, 48.31, 48.32, 48.33, 48.34, 48.35, 48.36, 48.37, 48.38, 48.39, 48.4, 48.41, 48.42, 48.43, 48.44, 48.45, 48.46, 48.47, 48.48, 48.49, 48.5, 48.51, 48.52, 48.53, 48.54, 48.55, 48.56, 48.57, 48.58, 48.59, 48.6, 48.61, 48.62, 48.63, 48.64, 48.65, 48.66, 48.67, 48.68, 48.69, 48.7, 48.71, 48.72, 48.73, 48.74, 48.75, 48.76, 48.77, 48.78, 48.79, 48.8, 48.81, 48.82, 48.83, 48.84, 48.85, 48.86, 48.87, 48.88, 48.89, 48.9, 48.91, 48.92, 48.93, 48.94, 48.95, 48.96, 48.97, 48.98, 48.99, 49, 49.01, 49.02, 49.03, 49.04, 49.05, 49.06, 49.07, 49.08, 49.09, 49.1, 49.11, 49.12, 49.13, 49.14, 49.15, 49.16, 49.17, 49.18, 49.19, 49.2, 49.21, 49.22, 49.23, 49.24, 49.25, 49.26, 49.27, 49.28, 49.29, 49.3, 49.31, 49.32, 49.33, 49.34, 49.35, 49.36, 49.37, 49.38, 49.39, 49.4, 49.41, 49.42, 49.43, 49.44, 49.45, 49.46, 49.47, 49.48, 49.49, 49.5, 49.51, 49.52, 49.53, 49.54, 49.55, 49.56, 49.57, 49.58, 49.59, 49.6, 49.61, 49.62, 49.63, 49.64, 49.65, 49.66, 49.67, 49.68, 49.69, 49.7, 49.71, 49.72, 49.73, 49.74, 49.75, 49.76, 49.77, 49.78, 49.79, 49.8, 49.81, 49.82, 49.83, 49.84, 49.85, 49.86, 49.87, 49.88, 49.89, 49.9, 49.91, 49.92, 49.93, 49.94, 49.95, 49.96, 49.97, 49.98, 49.99, 50, 50.01, 50.02, 50.03, 50.04, 50.05, 50.06, 50.07, 50.08, 50.09, 50.1, 50.11, 50.12, 50.13, 50.14, 50.15, 50.16, 50.17, 50.18, 50.19, 50.2, 50.21, 50.22, 50.23, 50.24, 50.25, 50.26, 50.27, 50.28, 50.29, 50.3, 50.31, 50.32, 50.33, 50.34, 50.35, 50.36, 50.37, 50.38, 50.39, 50.4, 50.41, 50.42, 50.43, 50.44, 50.45, 50.46, 50.47, 50.48, 50.49, 50.5, 50.51, 50.52, 50.53, 50.54, 50.55, 50.56, 50.57, 50.58, 50.59, 50.6, 50.61, 50.62, 50.63, 50.64, 50.65, 50.66, 50.67, 50.68, 50.69, 50.7, 50.71, 50.72, 50.73, 50.74, 50.75, 50.76, 50.77, 50.78, 50.79, 50.8, 50.81, 50.82, 50.83, 50.84, 50.85, 50.86, 50.87, 50.88, 50.89, 50.9, 50.91, 50.92, 50.93, 50.94, 50.95, 50.96, 50.97, 50.98, 50.99, 51, 51.01, 51.02, 51.03, 51.04, 51.05, 51.06, 51.07, 51.08, 51.09, 51.1, 51.11, 51.12, 51.13, 51.14, 51.15, 51.16, 51.17, 51.18, 51.19, 51.2, 51.21, 51.22, 51.23, 51.24, 51.25, 51.26, 51.27, 51.28, 51.29, 51.3, 51.31, 51.32, 51.33, 51.34, 51.35, 51.36, 51.37, 51.38, 51.39, 51.4, 51.41, 51.42, 51.43, 51.44, 51.45, 51.46, 51.47, 51.48, 51.49, 51.5, 51.51, 51.52, 51.53, 51.54, 51.55, 51.56, 51.57, 51.58, 51.59, 51.6, 51.61, 51.62, 51.63, 51.64, 51.65, 51.66, 51.67, 51.68, 51.69, 51.7, 51.71, 51.72, 51.73, 51.74, 51.75, 51.76, 51.77, 51.78, 51.79, 51.8, 51.81, 51.82, 51.83, 51.84, 51.85, 51.86, 51.87, 51.88, 51.89, 51.9, 51.91, 51.92, 51.93, 51.94, 51.95, 51.96, 51.97, 51.98, 51.99, 52, 52.01, 52.02, 52.03, 52.04, 52.05, 52.06, 52.07, 52.08, 52.09, 52.1, 52.11, 52.12, 52.13, 52.14, 52.15, 52.16, 52.17, 52.18, 52.19, 52.2, 52.21, 52.22, 52.23, 52.24, 52.25, 52.26, 52.27, 52.28, 52.29, 52.3, 52.31, 52.32, 52.33, 52.34, 52.35, 52.36, 52.37, 52.38, 52.39, 52.4, 52.41, 52.42, 52.43, 52.44, 52.45, 52.46, 52.47, 52.48, 52.49, 52.5, 52.51, 52.52, 52.53, 52.54, 52.55, 52.56, 52.57, 52.58, 52.59, 52.6, 52.61, 52.62, 52.63, 52.64, 52.65, 52.66, 52.67, 52.68, 52.69, 52.7, 52.71, 52.72, 52.73, 52.74, 52.75, 52.76, 52.77, 52.78, 52.79, 52.8, 52.81, 52.82, 52.83, 52.84, 52.85, 52.86, 52.87, 52.88, 52.89, 52.9, 52.91, 52.92, 52.93, 52.94, 52.95, 52.96, 52.97, 52.98, 52.99, 53, 53.01, 53.02, 53.03, 53.04, 53.05, 53.06, 53.07, 53.08, 53.09, 53.1, 53.11, 53.12, 53.13, 53.14, 53.15, 53.16, 53.17, 53.18, 53.19, 53.2, 53.21, 53.22, 53.23, 53.24, 53.25, 53.26, 53.27, 53.28, 53.29, 53.3, 53.31, 53.32, 53.33, 53.34, 53.35, 53.36, 53.37, 53.38, 53.39, 53.4, 53.41, 53.42, 53.43, 53.44, 53.45, 53.46, 53.47, 53.48, 53.49, 53.5, 53.51, 53.52, 53.53, 53.54, 53.55, 53.56, 53.57, 53.58, 53.59, 53.6, 53.61, 53.62, 53.63, 53.64, 53.65, 53.66, 53.67, 53.68, 53.69, 53.7, 53.71, 53.72, 53.73, 53.74, 53.75, 53.76, 53.77, 53.78, 53.79, 53.8, 53.81, 53.82, 53.83, 53.84, 53.85, 53.86, 53.87, 53.88, 53.89, 53.9, 53.91, 53.92, 53.93, 53.94, 53.95, 53.96, 53.97, 53.98, 53.99, 54, 54.01, 54.02, 54.03, 54.04, 54.05, 54.06, 54.07, 54.08, 54.09, 54.1, 54.11, 54.12, 54.13, 54.14, 54.15, 54.16, 54.17, 54.18, 54.19, 54.2, 54.21, 54.22, 54.23, 54.24, 54.25, 54.26, 54.27, 54.28, 54.29, 54.3, 54.31, 54.32, 54.33, 54.34, 54.35, 54.36, 54.37, 54.38, 54.39, 54.4, 54.41, 54.42, 54.43, 54.44, 54.45, 54.46, 54.47, 54.48, 54.49, 54.5, 54.51, 54.52, 54.53, 54.54, 54.55, 54.56, 54.57, 54.58, 54.59, 54.6, 54.61, 54.62, 54.63, 54.64, 54.65, 54.66, 54.67, 54.68, 54.69, 54.7, 54.71, 54.72, 54.73, 54.74, 54.75, 54.76, 54.77, 54.78, 54.79, 54.8, 54.81, 54.82, 54.83, 54.84, 54.85, 54.86, 54.87, 54.88, 54.89, 54.9, 54.91, 54.92, 54.93, 54.94, 54.95, 54.96, 54.97, 54.98, 54.99, 55, 55.01, 55.02, 55.03, 55.04, 55.05, 55.06, 55.07, 55.08, 55.09, 55.1, 55.11, 55.12, 55.13, 55.14, 55.15, 55.16, 55.17, 55.18, 55.19, 55.2, 55.21, 55.22, 55.23, 55.24, 55.25, 55.26, 55.27, 55.28, 55.29, 55.3, 55.31, 55.32, 55.33, 55.34, 55.35, 55.36, 55.37, 55.38, 55.39, 55.4, 55.41, 55.42, 55.43, 55.44, 55.45, 55.46, 55.47, 55.48, 55.49, 55.5, 55.51, 55.52, 55.53, 55.54, 55.55, 55.56, 55.57, 55.58, 55.59, 55.6, 55.61, 55.62, 55.63, 55.64, 55.65, 55.66, 55.67, 55.68, 55.69, 55.7, 55.71, 55.72, 55.73, 55.74, 55.75, 55.76, 55.77, 55.78, 55.79, 55.8, 55.81, 55.82, 55.83, 55.84, 55.85, 55.86, 55.87, 55.88, 55.89, 55.9, 55.91, 55.92, 55.93, 55.94, 55.95, 55.96, 55.97, 55.98, 55.99, 56, 56.01, 56.02, 56.03, 56.04, 56.05, 56.06, 56.07, 56.08, 56.09, 56.1, 56.11, 56.12, 56.13, 56.14, 56.15, 56.16, 56.17, 56.18, 56.19, 56.2, 56.21, 56.22, 56.23, 56.24, 56.25, 56.26, 56.27, 56.28, 56.29, 56.3, 56.31, 56.32, 56.33, 56.34, 56.35, 56.36, 56.37, 56.38, 56.39, 56.4, 56.41, 56.42, 56.43, 56.44, 56.45, 56.46, 56.47, 56.48, 56.49, 56.5, 56.51, 56.52, 56.53, 56.54, 56.55, 56.56, 56.57, 56.58, 56.59, 56.6, 56.61, 56.62, 56.63, 56.64, 56.65, 56.66, 56.67, 56.68, 56.69, 56.7, 56.71, 56.72, 56.73, 56.74, 56.75, 56.76, 56.77, 56.78, 56.79, 56.8, 56.81, 56.82, 56.83, 56.84, 56.85, 56.86, 56.87, 56.88, 56.89, 56.9, 56.91, 56.92, 56.93, 56.94, 56.95, 56.96, 56.97, 56.98, 56.99, 57, 57.01, 57.02, 57.03, 57.04, 57.05, 57.06, 57.07, 57.08, 57.09, 57.1, 57.11, 57.12, 57.13, 57.14, 57.15, 57.16, 57.17, 57.18, 57.19, 57.2, 57.21, 57.22, 57.23, 57.24, 57.25, 57.26, 57.27, 57.28, 57.29, 57.3, 57.31, 57.32, 57.33, 57.34, 57.35, 57.36, 57.37, 57.38, 57.39, 57.4, 57.41, 57.42, 57.43, 57.44, 57.45, 57.46, 57.47, 57.48, 57.49, 57.5, 57.51, 57.52, 57.53, 57.54, 57.55, 57.56, 57.57, 57.58, 57.59, 57.6, 57.61, 57.62, 57.63, 57.64, 57.65, 57.66, 57.67, 57.68, 57.69, 57.7, 57.71, 57.72, 57.73, 57.74, 57.75, 57.76, 57.77, 57.78, 57.79, 57.8, 57.81, 57.82, 57.83, 57.84, 57.85, 57.86, 57.87, 57.88, 57.89, 57.9, 57.91, 57.92, 57.93, 57.94, 57.95, 57.96, 57.97, 57.98, 57.99, 58, 58.01, 58.02, 58.03, 58.04, 58.05, 58.06, 58.07, 58.08, 58.09, 58.1, 58.11, 58.12, 58.13, 58.14, 58.15, 58.16, 58.17, 58.18, 58.19, 58.2, 58.21, 58.22, 58.23, 58.24, 58.25, 58.26, 58.27, 58.28, 58.29, 58.3, 58.31, 58.32, 58.33, 58.34, 58.35, 58.36, 58.37, 58.38, 58.39, 58.4, 58.41, 58.42, 58.43, 58.44, 58.45, 58.46, 58.47, 58.48, 58.49, 58.5, 58.51, 58.52, 58.53, 58.54, 58.55, 58.56, 58.57, 58.58, 58.59, 58.6, 58.61, 58.62, 58.63, 58.64, 58.65, 58.66, 58.67, 58.68, 58.69, 58.7, 58.71, 58.72, 58.73, 58.74, 58.75, 58.76, 58.77, 58.78, 58.79, 58.8, 58.81, 58.82, 58.83, 58.84, 58.85, 58.86, 58.87, 58.88, 58.89, 58.9, 58.91, 58.92, 58.93, 58.94, 58.95, 58.96, 58.97, 58.98, 58.99, 59, 59.01, 59.02, 59.03, 59.04, 59.05, 59.06, 59.07, 59.08, 59.09, 59.1, 59.11, 59.12, 59.13, 59.14, 59.15, 59.16, 59.17, 59.18, 59.19, 59.2, 59.21, 59.22, 59.23, 59.24, 59.25, 59.26, 59.27, 59.28, 59.29, 59.3, 59.31, 59.32, 59.33, 59.34, 59.35, 59.36, 59.37, 59.38, 59.39, 59.4, 59.41, 59.42, 59.43, 59.44, 59.45, 59.46, 59.47, 59.48, 59.49, 59.5, 59.51, 59.52, 59.53, 59.54, 59.55, 59.56, 59.57, 59.58, 59.59, 59.6, 59.61, 59.62, 59.63, 59.64, 59.65, 59.66, 59.67, 59.68, 59.69, 59.7, 59.71, 59.72, 59.73, 59.74, 59.75, 59.76, 59.77, 59.78, 59.79, 59.8, 59.81, 59.82, 59.83, 59.84, 59.85, 59.86, 59.87, 59.88, 59.89, 59.9, 59.91, 59.92, 59.93, 59.94, 59.95, 59.96, 59.97, 59.98, 59.99, 60, 60.01, 60.02, 60.03, 60.04, 60.05, 60.06, 60.07, 60.08, 60.09, 60.1, 60.11, 60.12, 60.13, 60.14, 60.15, 60.16, 60.17, 60.18, 60.19, 60.2, 60.21, 60.22, 60.23, 60.24, 60.25, 60.26, 60.27, 60.28, 60.29, 60.3, 60.31, 60.32, 60.33, 60.34, 60.35, 60.36, 60.37, 60.38, 60.39, 60.4, 60.41, 60.42, 60.43, 60.44, 60.45, 60.46, 60.47, 60.48, 60.49, 60.5, 60.51, 60.52, 60.53, 60.54, 60.55, 60.56, 60.57, 60.58, 60.59, 60.6, 60.61, 60.62, 60.63, 60.64, 60.65, 60.66, 60.67, 60.68, 60.69, 60.7, 60.71, 60.72, 60.73, 60.74, 60.75, 60.76, 60.77, 60.78, 60.79, 60.8, 60.81, 60.82, 60.83, 60.84, 60.85, 60.86, 60.87, 60.88, 60.89, 60.9, 60.91, 60.92, 60.93, 60.94, 60.95, 60.96, 60.97, 60.98, 60.99, 61, 61.01, 61.02, 61.03, 61.04, 61.05, 61.06, 61.07, 61.08, 61.09, 61.1, 61.11, 61.12, 61.13, 61.14, 61.15, 61.16, 61.17, 61.18, 61.19, 61.2, 61.21, 61.22, 61.23, 61.24, 61.25, 61.26, 61.27, 61.28, 61.29, 61.3, 61.31, 61.32, 61.33, 61.34, 61.35, 61.36, 61.37, 61.38, 61.39, 61.4, 61.41, 61.42, 61.43, 61.44, 61.45, 61.46, 61.47, 61.48, 61.49, 61.5, 61.51, 61.52, 61.53, 61.54, 61.55, 61.56, 61.57, 61.58, 61.59, 61.6, 61.61, 61.62, 61.63, 61.64, 61.65, 61.66, 61.67, 61.68, 61.69, 61.7, 61.71, 61.72, 61.73, 61.74, 61.75, 61.76, 61.77, 61.78, 61.79, 61.8, 61.81, 61.82, 61.83, 61.84, 61.85, 61.86, 61.87, 61.88, 61.89, 61.9, 61.91, 61.92, 61.93, 61.94, 61.95, 61.96, 61.97, 61.98, 61.99, 62, 62.01, 62.02, 62.03, 62.04, 62.05, 62.06, 62.07, 62.08, 62.09, 62.1, 62.11, 62.12, 62.13, 62.14, 62.15, 62.16, 62.17, 62.18, 62.19, 62.2, 62.21, 62.22, 62.23, 62.24, 62.25, 62.26, 62.27, 62.28, 62.29, 62.3, 62.31, 62.32, 62.33, 62.34, 62.35, 62.36, 62.37, 62.38, 62.39, 62.4, 62.41, 62.42, 62.43, 62.44, 62.45, 62.46, 62.47, 62.48, 62.49, 62.5, 62.51, 62.52, 62.53, 62.54, 62.55, 62.56, 62.57, 62.58, 62.59, 62.6, 62.61, 62.62, 62.63, 62.64, 62.65, 62.66, 62.67, 62.68, 62.69, 62.7, 62.71, 62.72, 62.73, 62.74, 62.75, 62.76, 62.77, 62.78, 62.79, 62.8, 62.81, 62.82, 62.83, 62.84, 62.85, 62.86, 62.87, 62.88, 62.89, 62.9, 62.91, 62.92, 62.93, 62.94, 62.95, 62.96, 62.97, 62.98, 62.99, 63, 63.01, 63.02, 63.03, 63.04, 63.05, 63.06, 63.07, 63.08, 63.09, 63.1, 63.11, 63.12, 63.13, 63.14, 63.15, 63.16, 63.17, 63.18, 63.19, 63.2, 63.21, 63.22, 63.23, 63.24, 63.25, 63.26, 63.27, 63.28, 63.29, 63.3, 63.31, 63.32, 63.33, 63.34, 63.35, 63.36, 63.37, 63.38, 63.39, 63.4, 63.41, 63.42, 63.43, 63.44, 63.45, 63.46, 63.47, 63.48, 63.49, 63.5, 63.51, 63.52, 63.53, 63.54, 63.55, 63.56, 63.57, 63.58, 63.59, 63.6, 63.61, 63.62, 63.63, 63.64, 63.65, 63.66, 63.67, 63.68, 63.69, 63.7, 63.71, 63.72, 63.73, 63.74, 63.75, 63.76, 63.77, 63.78, 63.79, 63.8, 63.81, 63.82, 63.83, 63.84, 63.85, 63.86, 63.87, 63.88, 63.89, 63.9, 63.91, 63.92, 63.93, 63.94, 63.95, 63.96, 63.97, 63.98, 63.99, 64, 64.01, 64.02, 64.03, 64.04, 64.05, 64.06, 64.07, 64.08, 64.09, 64.1, 64.11, 64.12, 64.13, 64.14, 64.15, 64.16, 64.17, 64.18, 64.19, 64.2, 64.21, 64.22, 64.23, 64.24, 64.25, 64.26, 64.27, 64.28, 64.29, 64.3, 64.31, 64.32, 64.33, 64.34, 64.35, 64.36, 64.37, 64.38, 64.39, 64.4, 64.41, 64.42, 64.43, 64.44, 64.45, 64.46, 64.47, 64.48, 64.49, 64.5, 64.51, 64.52, 64.53, 64.54, 64.55, 64.56, 64.57, 64.58, 64.59, 64.6, 64.61, 64.62, 64.63, 64.64, 64.65, 64.66, 64.67, 64.68, 64.69, 64.7, 64.71, 64.72, 64.73, 64.74, 64.75, 64.76, 64.77, 64.78, 64.79, 64.8, 64.81, 64.82, 64.83, 64.84, 64.85, 64.86, 64.87, 64.88, 64.89, 64.9, 64.91, 64.92, 64.93, 64.94, 64.95, 64.96, 64.97, 64.98, 64.99, 65, 65.01, 65.02, 65.03, 65.04, 65.05, 65.06, 65.07, 65.08, 65.09, 65.1, 65.11, 65.12, 65.13, 65.14, 65.15, 65.16, 65.17, 65.18, 65.19, 65.2, 65.21, 65.22, 65.23, 65.24, 65.25, 65.26, 65.27, 65.28, 65.29, 65.3, 65.31, 65.32, 65.33, 65.34, 65.35, 65.36, 65.37, 65.38, 65.39, 65.4, 65.41, 65.42, 65.43, 65.44, 65.45, 65.46, 65.47, 65.48, 65.49, 65.5, 65.51, 65.52, 65.53, 65.54, 65.55, 65.56, 65.57, 65.58, 65.59, 65.6, 65.61, 65.62, 65.63, 65.64, 65.65, 65.66, 65.67, 65.68, 65.69, 65.7, 65.71, 65.72, 65.73, 65.74, 65.75, 65.76, 65.77, 65.78, 65.79, 65.8, 65.81, 65.82, 65.83, 65.84, 65.85, 65.86, 65.87, 65.88, 65.89, 65.9, 65.91, 65.92, 65.93, 65.94, 65.95, 65.96, 65.97, 65.98, 65.99, 66, 66.01, 66.02, 66.03, 66.04, 66.05, 66.06, 66.07, 66.08, 66.09, 66.1, 66.11, 66.12, 66.13, 66.14, 66.15, 66.16, 66.17, 66.18, 66.19, 66.2, 66.21, 66.22, 66.23, 66.24, 66.25, 66.26, 66.27, 66.28, 66.29, 66.3, 66.31, 66.32, 66.33, 66.34, 66.35, 66.36, 66.37, 66.38, 66.39, 66.4, 66.41, 66.42, 66.43, 66.44, 66.45, 66.46, 66.47, 66.48, 66.49, 66.5, 66.51, 66.52, 66.53, 66.54, 66.55, 66.56, 66.57, 66.58, 66.59, 66.6, 66.61, 66.62, 66.63, 66.64, 66.65, 66.66, 66.67, 66.68, 66.69, 66.7, 66.71, 66.72, 66.73, 66.74, 66.75, 66.76, 66.77, 66.78, 66.79, 66.8, 66.81, 66.82, 66.83, 66.84, 66.85, 66.86, 66.87, 66.88, 66.89, 66.9, 66.91, 66.92, 66.93, 66.94, 66.95, 66.96, 66.97, 66.98, 66.99, 67, 67.01, 67.02, 67.03, 67.04, 67.05, 67.06, 67.07, 67.08, 67.09, 67.1, 67.11, 67.12, 67.13, 67.14, 67.15, 67.16, 67.17, 67.18, 67.19, 67.2, 67.21, 67.22, 67.23, 67.24, 67.25, 67.26, 67.27, 67.28, 67.29, 67.3, 67.31, 67.32, 67.33, 67.34, 67.35, 67.36, 67.37, 67.38, 67.39, 67.4, 67.41, 67.42, 67.43, 67.44, 67.45, 67.46, 67.47, 67.48, 67.49, 67.5, 67.51, 67.52, 67.53, 67.54, 67.55, 67.56, 67.57, 67.58, 67.59, 67.6, 67.61, 67.62, 67.63, 67.64, 67.65, 67.66, 67.67, 67.68, 67.69, 67.7, 67.71, 67.72, 67.73, 67.74, 67.75, 67.76, 67.77, 67.78, 67.79, 67.8, 67.81, 67.82, 67.83, 67.84, 67.85, 67.86, 67.87, 67.88, 67.89, 67.9, 67.91, 67.92, 67.93, 67.94, 67.95, 67.96, 67.97, 67.98, 67.99, 68, 68.01, 68.02, 68.03, 68.04, 68.05, 68.06, 68.07, 68.08, 68.09, 68.1, 68.11, 68.12, 68.13, 68.14, 68.15, 68.16, 68.17, 68.18, 68.19, 68.2, 68.21, 68.22, 68.23, 68.24, 68.25, 68.26, 68.27, 68.28, 68.29, 68.3, 68.31, 68.32, 68.33, 68.34, 68.35, 68.36, 68.37, 68.38, 68.39, 68.4, 68.41, 68.42, 68.43, 68.44, 68.45, 68.46, 68.47, 68.48, 68.49, 68.5, 68.51, 68.52, 68.53, 68.54, 68.55, 68.56, 68.57, 68.58, 68.59, 68.6, 68.61, 68.62, 68.63, 68.64, 68.65, 68.66, 68.67, 68.68, 68.69, 68.7, 68.71, 68.72, 68.73, 68.74, 68.75, 68.76, 68.77, 68.78, 68.79, 68.8, 68.81, 68.82, 68.83, 68.84, 68.85, 68.86, 68.87, 68.88, 68.89, 68.9, 68.91, 68.92, 68.93, 68.94, 68.95, 68.96, 68.97, 68.98, 68.99, 69, 69.01, 69.02, 69.03, 69.04, 69.05, 69.06, 69.07, 69.08, 69.09, 69.1, 69.11, 69.12, 69.13, 69.14, 69.15, 69.16, 69.17, 69.18, 69.19, 69.2, 69.21, 69.22, 69.23, 69.24, 69.25, 69.26, 69.27, 69.28, 69.29, 69.3, 69.31, 69.32, 69.33, 69.34, 69.35, 69.36, 69.37, 69.38, 69.39, 69.4, 69.41, 69.42, 69.43, 69.44, 69.45, 69.46, 69.47, 69.48, 69.49, 69.5, 69.51, 69.52, 69.53, 69.54, 69.55, 69.56, 69.57, 69.58, 69.59, 69.6, 69.61, 69.62, 69.63, 69.64, 69.65, 69.66, 69.67, 69.68, 69.69, 69.7, 69.71, 69.72, 69.73, 69.74, 69.75, 69.76, 69.77, 69.78, 69.79, 69.8, 69.81, 69.82, 69.83, 69.84, 69.85, 69.86, 69.87, 69.88, 69.89, 69.9, 69.91, 69.92, 69.93, 69.94, 69.95, 69.96, 69.97, 69.98, 69.99, 70, 70.01, 70.02, 70.03, 70.04, 70.05, 70.06, 70.07, 70.08, 70.09, 70.1, 70.11, 70.12, 70.13, 70.14, 70.15, 70.16, 70.17, 70.18, 70.19, 70.2, 70.21, 70.22, 70.23, 70.24, 70.25, 70.26, 70.27, 70.28, 70.29, 70.3, 70.31, 70.32, 70.33, 70.34, 70.35, 70.36, 70.37, 70.38, 70.39, 70.4, 70.41, 70.42, 70.43, 70.44, 70.45, 70.46, 70.47, 70.48, 70.49, 70.5, 70.51, 70.52, 70.53, 70.54, 70.55, 70.56, 70.57, 70.58, 70.59, 70.6, 70.61, 70.62, 70.63, 70.64, 70.65, 70.66, 70.67, 70.68, 70.69, 70.7, 70.71, 70.72, 70.73, 70.74, 70.75, 70.76, 70.77, 70.78, 70.79, 70.8, 70.81, 70.82, 70.83, 70.84, 70.85, 70.86, 70.87, 70.88, 70.89, 70.9, 70.91, 70.92, 70.93, 70.94, 70.95, 70.96, 70.97, 70.98, 70.99, 71, 71.01, 71.02, 71.03, 71.04, 71.05, 71.06, 71.07, 71.08, 71.09, 71.1, 71.11, 71.12, 71.13, 71.14, 71.15, 71.16, 71.17, 71.18, 71.19, 71.2, 71.21, 71.22, 71.23, 71.24, 71.25, 71.26, 71.27, 71.28, 71.29, 71.3, 71.31, 71.32, 71.33, 71.34, 71.35, 71.36, 71.37, 71.38, 71.39, 71.4, 71.41, 71.42, 71.43, 71.44, 71.45, 71.46, 71.47, 71.48, 71.49, 71.5, 71.51, 71.52, 71.53, 71.54, 71.55, 71.56, 71.57, 71.58, 71.59, 71.6, 71.61, 71.62, 71.63, 71.64, 71.65, 71.66, 71.67, 71.68, 71.69, 71.7, 71.71, 71.72, 71.73, 71.74, 71.75, 71.76, 71.77, 71.78, 71.79, 71.8, 71.81, 71.82, 71.83, 71.84, 71.85, 71.86, 71.87, 71.88, 71.89, 71.9, 71.91, 71.92, 71.93, 71.94, 71.95, 71.96, 71.97, 71.98, 71.99, 72, 72.01, 72.02, 72.03, 72.04, 72.05, 72.06, 72.07, 72.08, 72.09, 72.1, 72.11, 72.12, 72.13, 72.14, 72.15, 72.16, 72.17, 72.18, 72.19, 72.2, 72.21, 72.22, 72.23, 72.24, 72.25, 72.26, 72.27, 72.28, 72.29, 72.3, 72.31, 72.32, 72.33, 72.34, 72.35, 72.36, 72.37, 72.38, 72.39, 72.4, 72.41, 72.42, 72.43, 72.44, 72.45, 72.46, 72.47, 72.48, 72.49, 72.5, 72.51, 72.52, 72.53, 72.54, 72.55, 72.56, 72.57, 72.58, 72.59, 72.6, 72.61, 72.62, 72.63, 72.64, 72.65, 72.66, 72.67, 72.68, 72.69, 72.7, 72.71, 72.72, 72.73, 72.74, 72.75, 72.76, 72.77, 72.78, 72.79, 72.8, 72.81, 72.82, 72.83, 72.84, 72.85, 72.86, 72.87, 72.88, 72.89, 72.9, 72.91, 72.92, 72.93, 72.94, 72.95, 72.96, 72.97, 72.98, 72.99, 73, 73.01, 73.02, 73.03, 73.04, 73.05, 73.06, 73.07, 73.08, 73.09, 73.1, 73.11, 73.12, 73.13, 73.14, 73.15, 73.16, 73.17, 73.18, 73.19, 73.2, 73.21, 73.22, 73.23, 73.24, 73.25, 73.26, 73.27, 73.28, 73.29, 73.3, 73.31, 73.32, 73.33, 73.34, 73.35, 73.36, 73.37, 73.38, 73.39, 73.4, 73.41, 73.42, 73.43, 73.44, 73.45, 73.46, 73.47, 73.48, 73.49, 73.5, 73.51, 73.52, 73.53, 73.54, 73.55, 73.56, 73.57, 73.58, 73.59, 73.6, 73.61, 73.62, 73.63, 73.64, 73.65, 73.66, 73.67, 73.68, 73.69, 73.7, 73.71, 73.72, 73.73, 73.74, 73.75, 73.76, 73.77, 73.78, 73.79, 73.8, 73.81, 73.82, 73.83, 73.84, 73.85, 73.86, 73.87, 73.88, 73.89, 73.9, 73.91, 73.92, 73.93, 73.94, 73.95, 73.96, 73.97, 73.98, 73.99, 74, 74.01, 74.02, 74.03, 74.04, 74.05, 74.06, 74.07, 74.08, 74.09, 74.1, 74.11, 74.12, 74.13, 74.14, 74.15, 74.16, 74.17, 74.18, 74.19, 74.2, 74.21, 74.22, 74.23, 74.24, 74.25, 74.26, 74.27, 74.28, 74.29, 74.3, 74.31, 74.32, 74.33, 74.34, 74.35, 74.36, 74.37, 74.38, 74.39, 74.4, 74.41, 74.42, 74.43, 74.44, 74.45, 74.46, 74.47, 74.48, 74.49, 74.5, 74.51, 74.52, 74.53, 74.54, 74.55, 74.56, 74.57, 74.58, 74.59, 74.6, 74.61, 74.62, 74.63, 74.64, 74.65, 74.66, 74.67, 74.68, 74.69, 74.7, 74.71, 74.72, 74.73, 74.74, 74.75, 74.76, 74.77, 74.78, 74.79, 74.8, 74.81, 74.82, 74.83, 74.84, 74.85, 74.86, 74.87, 74.88, 74.89, 74.9, 74.91, 74.92, 74.93, 74.94, 74.95, 74.96, 74.97, 74.98, 74.99, 75, 75.01, 75.02, 75.03, 75.04, 75.05, 75.06, 75.07, 75.08, 75.09, 75.1, 75.11, 75.12, 75.13, 75.14, 75.15, 75.16, 75.17, 75.18, 75.19, 75.2, 75.21, 75.22, 75.23, 75.24, 75.25, 75.26, 75.27, 75.28, 75.29, 75.3, 75.31, 75.32, 75.33, 75.34, 75.35, 75.36, 75.37, 75.38, 75.39, 75.4, 75.41, 75.42, 75.43, 75.44, 75.45, 75.46, 75.47, 75.48, 75.49, 75.5, 75.51, 75.52, 75.53, 75.54, 75.55, 75.56, 75.57, 75.58, 75.59, 75.6, 75.61, 75.62, 75.63, 75.64, 75.65, 75.66, 75.67, 75.68, 75.69, 75.7, 75.71, 75.72, 75.73, 75.74, 75.75, 75.76, 75.77, 75.78, 75.79, 75.8, 75.81, 75.82, 75.83, 75.84, 75.85, 75.86, 75.87, 75.88, 75.89, 75.9, 75.91, 75.92, 75.93, 75.94, 75.95, 75.96, 75.97, 75.98, 75.99, 76, 76.01, 76.02, 76.03, 76.04, 76.05, 76.06, 76.07, 76.08, 76.09, 76.1, 76.11, 76.12, 76.13, 76.14, 76.15, 76.16, 76.17, 76.18, 76.19, 76.2, 76.21, 76.22, 76.23, 76.24, 76.25, 76.26, 76.27, 76.28, 76.29, 76.3, 76.31, 76.32, 76.33, 76.34, 76.35, 76.36, 76.37, 76.38, 76.39, 76.4, 76.41, 76.42, 76.43, 76.44, 76.45, 76.46, 76.47, 76.48, 76.49, 76.5, 76.51, 76.52, 76.53, 76.54, 76.55, 76.56, 76.57, 76.58, 76.59, 76.6, 76.61, 76.62, 76.63, 76.64, 76.65, 76.66, 76.67, 76.68, 76.69, 76.7, 76.71, 76.72, 76.73, 76.74, 76.75, 76.76, 76.77, 76.78, 76.79, 76.8, 76.81, 76.82, 76.83, 76.84, 76.85, 76.86, 76.87, 76.88, 76.89, 76.9, 76.91, 76.92, 76.93, 76.94, 76.95, 76.96, 76.97, 76.98, 76.99, 77, 77.01, 77.02, 77.03, 77.04, 77.05, 77.06, 77.07, 77.08, 77.09, 77.1, 77.11, 77.12, 77.13, 77.14, 77.15, 77.16, 77.17, 77.18, 77.19, 77.2, 77.21, 77.22, 77.23, 77.24, 77.25, 77.26, 77.27, 77.28, 77.29, 77.3, 77.31, 77.32, 77.33, 77.34, 77.35, 77.36, 77.37, 77.38, 77.39, 77.4, 77.41, 77.42, 77.43, 77.44, 77.45, 77.46, 77.47, 77.48, 77.49, 77.5, 77.51, 77.52, 77.53, 77.54, 77.55, 77.56, 77.57, 77.58, 77.59, 77.6, 77.61, 77.62, 77.63, 77.64, 77.65, 77.66, 77.67, 77.68, 77.69, 77.7, 77.71, 77.72, 77.73, 77.74, 77.75, 77.76, 77.77, 77.78, 77.79, 77.8, 77.81, 77.82, 77.83, 77.84, 77.85, 77.86, 77.87, 77.88, 77.89, 77.9, 77.91, 77.92, 77.93, 77.94, 77.95, 77.96, 77.97, 77.98, 77.99, 78, 78.01, 78.02, 78.03, 78.04, 78.05, 78.06, 78.07, 78.08, 78.09, 78.1, 78.11, 78.12, 78.13, 78.14, 78.15, 78.16, 78.17, 78.18, 78.19, 78.2, 78.21, 78.22, 78.23, 78.24, 78.25, 78.26, 78.27, 78.28, 78.29, 78.3, 78.31, 78.32, 78.33, 78.34, 78.35, 78.36, 78.37, 78.38, 78.39, 78.4, 78.41, 78.42, 78.43, 78.44, 78.45, 78.46, 78.47, 78.48, 78.49, 78.5, 78.51, 78.52, 78.53, 78.54, 78.55, 78.56, 78.57, 78.58, 78.59, 78.6, 78.61, 78.62, 78.63, 78.64, 78.65, 78.66, 78.67, 78.68, 78.69, 78.7, 78.71, 78.72, 78.73, 78.74, 78.75, 78.76, 78.77, 78.78, 78.79, 78.8, 78.81, 78.82, 78.83, 78.84, 78.85, 78.86, 78.87, 78.88, 78.89, 78.9, 78.91, 78.92, 78.93, 78.94, 78.95, 78.96, 78.97, 78.98, 78.99, 79, 79.01, 79.02, 79.03, 79.04, 79.05, 79.06, 79.07, 79.08, 79.09, 79.1, 79.11, 79.12, 79.13, 79.14, 79.15, 79.16, 79.17, 79.18, 79.19, 79.2, 79.21, 79.22, 79.23, 79.24, 79.25, 79.26, 79.27, 79.28, 79.29, 79.3, 79.31, 79.32, 79.33, 79.34, 79.35, 79.36, 79.37, 79.38, 79.39, 79.4, 79.41, 79.42, 79.43, 79.44, 79.45, 79.46, 79.47, 79.48, 79.49, 79.5, 79.51, 79.52, 79.53, 79.54, 79.55, 79.56, 79.57, 79.58, 79.59, 79.6, 79.61, 79.62, 79.63, 79.64, 79.65, 79.66, 79.67, 79.68, 79.69, 79.7, 79.71, 79.72, 79.73, 79.74, 79.75, 79.76, 79.77, 79.78, 79.79, 79.8, 79.81, 79.82, 79.83, 79.84, 79.85, 79.86, 79.87, 79.88, 79.89, 79.9, 79.91, 79.92, 79.93, 79.94, 79.95, 79.96, 79.97, 79.98, 79.99, 80, 80.01, 80.02, 80.03, 80.04, 80.05, 80.06, 80.07, 80.08, 80.09, 80.1, 80.11, 80.12, 80.13, 80.14, 80.15, 80.16, 80.17, 80.18, 80.19, 80.2, 80.21, 80.22, 80.23, 80.24, 80.25, 80.26, 80.27, 80.28, 80.29, 80.3, 80.31, 80.32, 80.33, 80.34, 80.35, 80.36, 80.37, 80.38, 80.39, 80.4, 80.41, 80.42, 80.43, 80.44, 80.45, 80.46, 80.47, 80.48, 80.49, 80.5, 80.51, 80.52, 80.53, 80.54, 80.55, 80.56, 80.57, 80.58, 80.59, 80.6, 80.61, 80.62, 80.63, 80.64, 80.65, 80.66, 80.67, 80.68, 80.69, 80.7, 80.71, 80.72, 80.73, 80.74, 80.75, 80.76, 80.77, 80.78, 80.79, 80.8, 80.81, 80.82, 80.83, 80.84, 80.85, 80.86, 80.87, 80.88, 80.89, 80.9, 80.91, 80.92, 80.93, 80.94, 80.95, 80.96, 80.97, 80.98, 80.99, 81, 81.01, 81.02, 81.03, 81.04, 81.05, 81.06, 81.07, 81.08, 81.09, 81.1, 81.11, 81.12, 81.13, 81.14, 81.15, 81.16, 81.17, 81.18, 81.19, 81.2, 81.21, 81.22, 81.23, 81.24, 81.25, 81.26, 81.27, 81.28, 81.29, 81.3, 81.31, 81.32, 81.33, 81.34, 81.35, 81.36, 81.37, 81.38, 81.39, 81.4, 81.41, 81.42, 81.43, 81.44, 81.45, 81.46, 81.47, 81.48, 81.49, 81.5, 81.51, 81.52, 81.53, 81.54, 81.55, 81.56, 81.57, 81.58, 81.59, 81.6, 81.61, 81.62, 81.63, 81.64, 81.65, 81.66, 81.67, 81.68, 81.69, 81.7, 81.71, 81.72, 81.73, 81.74, 81.75, 81.76, 81.77, 81.78, 81.79, 81.8, 81.81, 81.82, 81.83, 81.84, 81.85, 81.86, 81.87, 81.88, 81.89, 81.9, 81.91, 81.92, 81.93, 81.94, 81.95, 81.96, 81.97, 81.98, 81.99, 82, 82.01, 82.02, 82.03, 82.04, 82.05, 82.06, 82.07, 82.08, 82.09, 82.1, 82.11, 82.12, 82.13, 82.14, 82.15, 82.16, 82.17, 82.18, 82.19, 82.2, 82.21, 82.22, 82.23, 82.24, 82.25, 82.26, 82.27, 82.28, 82.29, 82.3, 82.31, 82.32, 82.33, 82.34, 82.35, 82.36, 82.37, 82.38, 82.39, 82.4, 82.41, 82.42, 82.43, 82.44, 82.45, 82.46, 82.47, 82.48, 82.49, 82.5, 82.51, 82.52, 82.53, 82.54, 82.55, 82.56, 82.57, 82.58, 82.59, 82.6, 82.61, 82.62, 82.63, 82.64, 82.65, 82.66, 82.67, 82.68, 82.69, 82.7, 82.71, 82.72, 82.73, 82.74, 82.75, 82.76, 82.77, 82.78, 82.79, 82.8, 82.81, 82.82, 82.83, 82.84, 82.85, 82.86, 82.87, 82.88, 82.89, 82.9, 82.91, 82.92, 82.93, 82.94, 82.95, 82.96, 82.97, 82.98, 82.99, 83, 83.01, 83.02, 83.03, 83.04, 83.05, 83.06, 83.07, 83.08, 83.09, 83.1, 83.11, 83.12, 83.13, 83.14, 83.15, 83.16, 83.17, 83.18, 83.19, 83.2, 83.21, 83.22, 83.23, 83.24, 83.25, 83.26, 83.27, 83.28, 83.29, 83.3, 83.31, 83.32, 83.33, 83.34, 83.35, 83.36, 83.37, 83.38, 83.39, 83.4, 83.41, 83.42, 83.43, 83.44, 83.45, 83.46, 83.47, 83.48, 83.49, 83.5, 83.51, 83.52, 83.53, 83.54, 83.55, 83.56, 83.57, 83.58, 83.59, 83.6, 83.61, 83.62, 83.63, 83.64, 83.65, 83.66, 83.67, 83.68, 83.69, 83.7, 83.71, 83.72, 83.73, 83.74, 83.75, 83.76, 83.77, 83.78, 83.79, 83.8, 83.81, 83.82, 83.83, 83.84, 83.85, 83.86, 83.87, 83.88, 83.89, 83.9, 83.91, 83.92, 83.93, 83.94, 83.95, 83.96, 83.97, 83.98, 83.99, 84, 84.01, 84.02, 84.03, 84.04, 84.05, 84.06, 84.07, 84.08, 84.09, 84.1, 84.11, 84.12, 84.13, 84.14, 84.15, 84.16, 84.17, 84.18, 84.19, 84.2, 84.21, 84.22, 84.23, 84.24, 84.25, 84.26, 84.27, 84.28, 84.29, 84.3, 84.31, 84.32, 84.33, 84.34, 84.35, 84.36, 84.37, 84.38, 84.39, 84.4, 84.41, 84.42, 84.43, 84.44, 84.45, 84.46, 84.47, 84.48, 84.49, 84.5, 84.51, 84.52, 84.53, 84.54, 84.55, 84.56, 84.57, 84.58, 84.59, 84.6, 84.61, 84.62, 84.63, 84.64, 84.65, 84.66, 84.67, 84.68, 84.69, 84.7, 84.71, 84.72, 84.73, 84.74, 84.75, 84.76, 84.77, 84.78, 84.79, 84.8, 84.81, 84.82, 84.83, 84.84, 84.85, 84.86, 84.87, 84.88, 84.89, 84.9, 84.91, 84.92, 84.93, 84.94, 84.95, 84.96, 84.97, 84.98, 84.99, 85, 85.01, 85.02, 85.03, 85.04, 85.05, 85.06, 85.07, 85.08, 85.09, 85.1, 85.11, 85.12, 85.13, 85.14, 85.15, 85.16, 85.17, 85.18, 85.19, 85.2, 85.21, 85.22, 85.23, 85.24, 85.25, 85.26, 85.27, 85.28, 85.29, 85.3, 85.31, 85.32, 85.33, 85.34, 85.35, 85.36, 85.37, 85.38, 85.39, 85.4, 85.41, 85.42, 85.43, 85.44, 85.45, 85.46, 85.47, 85.48, 85.49, 85.5, 85.51, 85.52, 85.53, 85.54, 85.55, 85.56, 85.57, 85.58, 85.59, 85.6, 85.61, 85.62, 85.63, 85.64, 85.65, 85.66, 85.67, 85.68, 85.69, 85.7, 85.71, 85.72, 85.73, 85.74, 85.75, 85.76, 85.77, 85.78, 85.79, 85.8, 85.81, 85.82, 85.83, 85.84, 85.85, 85.86, 85.87, 85.88, 85.89, 85.9, 85.91, 85.92, 85.93, 85.94, 85.95, 85.96, 85.97, 85.98, 85.99, 86, 86.01, 86.02, 86.03, 86.04, 86.05, 86.06, 86.07, 86.08, 86.09, 86.1, 86.11, 86.12, 86.13, 86.14, 86.15, 86.16, 86.17, 86.18, 86.19, 86.2, 86.21, 86.22, 86.23, 86.24, 86.25, 86.26, 86.27, 86.28, 86.29, 86.3, 86.31, 86.32, 86.33, 86.34, 86.35, 86.36, 86.37, 86.38, 86.39, 86.4, 86.41, 86.42, 86.43, 86.44, 86.45, 86.46, 86.47, 86.48, 86.49, 86.5, 86.51, 86.52, 86.53, 86.54, 86.55, 86.56, 86.57, 86.58, 86.59, 86.6, 86.61, 86.62, 86.63, 86.64, 86.65, 86.66, 86.67, 86.68, 86.69, 86.7, 86.71, 86.72, 86.73, 86.74, 86.75, 86.76, 86.77, 86.78, 86.79, 86.8, 86.81, 86.82, 86.83, 86.84, 86.85, 86.86, 86.87, 86.88, 86.89, 86.9, 86.91, 86.92, 86.93, 86.94, 86.95, 86.96, 86.97, 86.98, 86.99, 87, 87.01, 87.02, 87.03, 87.04, 87.05, 87.06, 87.07, 87.08, 87.09, 87.1, 87.11, 87.12, 87.13, 87.14, 87.15, 87.16, 87.17, 87.18, 87.19, 87.2, 87.21, 87.22, 87.23, 87.24, 87.25, 87.26, 87.27, 87.28, 87.29, 87.3, 87.31, 87.32, 87.33, 87.34, 87.35, 87.36, 87.37, 87.38, 87.39, 87.4, 87.41, 87.42, 87.43, 87.44, 87.45, 87.46, 87.47, 87.48, 87.49, 87.5, 87.51, 87.52, 87.53, 87.54, 87.55, 87.56, 87.57, 87.58, 87.59, 87.6, 87.61, 87.62, 87.63, 87.64, 87.65, 87.66, 87.67, 87.68, 87.69, 87.7, 87.71, 87.72, 87.73, 87.74, 87.75, 87.76, 87.77, 87.78, 87.79, 87.8, 87.81, 87.82, 87.83, 87.84, 87.85, 87.86, 87.87, 87.88, 87.89, 87.9, 87.91, 87.92, 87.93, 87.94, 87.95, 87.96, 87.97, 87.98, 87.99, 88, 88.01, 88.02, 88.03, 88.04, 88.05, 88.06, 88.07, 88.08, 88.09, 88.1, 88.11, 88.12, 88.13, 88.14, 88.15, 88.16, 88.17, 88.18, 88.19, 88.2, 88.21, 88.22, 88.23, 88.24, 88.25, 88.26, 88.27, 88.28, 88.29, 88.3, 88.31, 88.32, 88.33, 88.34, 88.35, 88.36, 88.37, 88.38, 88.39, 88.4, 88.41, 88.42, 88.43, 88.44, 88.45, 88.46, 88.47, 88.48, 88.49, 88.5, 88.51, 88.52, 88.53, 88.54, 88.55, 88.56, 88.57, 88.58, 88.59, 88.6, 88.61, 88.62, 88.63, 88.64, 88.65, 88.66, 88.67, 88.68, 88.69, 88.7, 88.71, 88.72, 88.73, 88.74, 88.75, 88.76, 88.77, 88.78, 88.79, 88.8, 88.81, 88.82, 88.83, 88.84, 88.85, 88.86, 88.87, 88.88, 88.89, 88.9, 88.91, 88.92, 88.93, 88.94, 88.95, 88.96, 88.97, 88.98, 88.99, 89, 89.01, 89.02, 89.03, 89.04, 89.05, 89.06, 89.07, 89.08, 89.09, 89.1, 89.11, 89.12, 89.13, 89.14, 89.15, 89.16, 89.17, 89.18, 89.19, 89.2, 89.21, 89.22, 89.23, 89.24, 89.25, 89.26, 89.27, 89.28, 89.29, 89.3, 89.31, 89.32, 89.33, 89.34, 89.35, 89.36, 89.37, 89.38, 89.39, 89.4, 89.41, 89.42, 89.43, 89.44, 89.45, 89.46, 89.47, 89.48, 89.49, 89.5, 89.51, 89.52, 89.53, 89.54, 89.55, 89.56, 89.57, 89.58, 89.59, 89.6, 89.61, 89.62, 89.63, 89.64, 89.65, 89.66, 89.67, 89.68, 89.69, 89.7, 89.71, 89.72, 89.73, 89.74, 89.75, 89.76, 89.77, 89.78, 89.79, 89.8, 89.81, 89.82, 89.83, 89.84, 89.85, 89.86, 89.87, 89.88, 89.89, 89.9, 89.91, 89.92, 89.93, 89.94, 89.95, 89.96, 89.97, 89.98, 89.99, 90, 90.01, 90.02, 90.03, 90.04, 90.05, 90.06, 90.07, 90.08, 90.09, 90.1, 90.11, 90.12, 90.13, 90.14, 90.15, 90.16, 90.17, 90.18, 90.19, 90.2, 90.21, 90.22, 90.23, 90.24, 90.25, 90.26, 90.27, 90.28, 90.29, 90.3, 90.31, 90.32, 90.33, 90.34, 90.35, 90.36, 90.37, 90.38, 90.39, 90.4, 90.41, 90.42, 90.43, 90.44, 90.45, 90.46, 90.47, 90.48, 90.49, 90.5, 90.51, 90.52, 90.53, 90.54, 90.55, 90.56, 90.57, 90.58, 90.59, 90.6, 90.61, 90.62, 90.63, 90.64, 90.65, 90.66, 90.67, 90.68, 90.69, 90.7, 90.71, 90.72, 90.73, 90.74, 90.75, 90.76, 90.77, 90.78, 90.79, 90.8, 90.81, 90.82, 90.83, 90.84, 90.85, 90.86, 90.87, 90.88, 90.89, 90.9, 90.91, 90.92, 90.93, 90.94, 90.95, 90.96, 90.97, 90.98, 90.99, 91, 91.01, 91.02, 91.03, 91.04, 91.05, 91.06, 91.07, 91.08, 91.09, 91.1, 91.11, 91.12, 91.13, 91.14, 91.15, 91.16, 91.17, 91.18, 91.19, 91.2, 91.21, 91.22, 91.23, 91.24, 91.25, 91.26, 91.27, 91.28, 91.29, 91.3, 91.31, 91.32, 91.33, 91.34, 91.35, 91.36, 91.37, 91.38, 91.39, 91.4, 91.41, 91.42, 91.43, 91.44, 91.45, 91.46, 91.47, 91.48, 91.49, 91.5, 91.51, 91.52, 91.53, 91.54, 91.55, 91.56, 91.57, 91.58, 91.59, 91.6, 91.61, 91.62, 91.63, 91.64, 91.65, 91.66, 91.67, 91.68, 91.69, 91.7, 91.71, 91.72, 91.73, 91.74, 91.75, 91.76, 91.77, 91.78, 91.79, 91.8, 91.81, 91.82, 91.83, 91.84, 91.85, 91.86, 91.87, 91.88, 91.89, 91.9, 91.91, 91.92, 91.93, 91.94, 91.95, 91.96, 91.97, 91.98, 91.99, 92, 92.01, 92.02, 92.03, 92.04, 92.05, 92.06, 92.07, 92.08, 92.09, 92.1, 92.11, 92.12, 92.13, 92.14, 92.15, 92.16, 92.17, 92.18, 92.19, 92.2, 92.21, 92.22, 92.23, 92.24, 92.25, 92.26, 92.27, 92.28, 92.29, 92.3, 92.31, 92.32, 92.33, 92.34, 92.35, 92.36, 92.37, 92.38, 92.39, 92.4, 92.41, 92.42, 92.43, 92.44, 92.45, 92.46, 92.47, 92.48, 92.49, 92.5, 92.51, 92.52, 92.53, 92.54, 92.55, 92.56, 92.57, 92.58, 92.59, 92.6, 92.61, 92.62, 92.63, 92.64, 92.65, 92.66, 92.67, 92.68, 92.69, 92.7, 92.71, 92.72, 92.73, 92.74, 92.75, 92.76, 92.77, 92.78, 92.79, 92.8, 92.81, 92.82, 92.83, 92.84, 92.85, 92.86, 92.87, 92.88, 92.89, 92.9, 92.91, 92.92, 92.93, 92.94, 92.95, 92.96, 92.97, 92.98, 92.99, 93, 93.01, 93.02, 93.03, 93.04, 93.05, 93.06, 93.07, 93.08, 93.09, 93.1, 93.11, 93.12, 93.13, 93.14, 93.15, 93.16, 93.17, 93.18, 93.19, 93.2, 93.21, 93.22, 93.23, 93.24, 93.25, 93.26, 93.27, 93.28, 93.29, 93.3, 93.31, 93.32, 93.33, 93.34, 93.35, 93.36, 93.37, 93.38, 93.39, 93.4, 93.41, 93.42, 93.43, 93.44, 93.45, 93.46, 93.47, 93.48, 93.49, 93.5, 93.51, 93.52, 93.53, 93.54, 93.55, 93.56, 93.57, 93.58, 93.59, 93.6, 93.61, 93.62, 93.63, 93.64, 93.65, 93.66, 93.67, 93.68, 93.69, 93.7, 93.71, 93.72, 93.73, 93.74, 93.75, 93.76, 93.77, 93.78, 93.79, 93.8, 93.81, 93.82, 93.83, 93.84, 93.85, 93.86, 93.87, 93.88, 93.89, 93.9, 93.91, 93.92, 93.93, 93.94, 93.95, 93.96, 93.97, 93.98, 93.99, 94, 94.01, 94.02, 94.03, 94.04, 94.05, 94.06, 94.07, 94.08, 94.09, 94.1, 94.11, 94.12, 94.13, 94.14, 94.15, 94.16, 94.17, 94.18, 94.19, 94.2, 94.21, 94.22, 94.23, 94.24, 94.25, 94.26, 94.27, 94.28, 94.29, 94.3, 94.31, 94.32, 94.33, 94.34, 94.35, 94.36, 94.37, 94.38, 94.39, 94.4, 94.41, 94.42, 94.43, 94.44, 94.45, 94.46, 94.47, 94.48, 94.49, 94.5, 94.51, 94.52, 94.53, 94.54, 94.55, 94.56, 94.57, 94.58, 94.59, 94.6, 94.61, 94.62, 94.63, 94.64, 94.65, 94.66, 94.67, 94.68, 94.69, 94.7, 94.71, 94.72, 94.73, 94.74, 94.75, 94.76, 94.77, 94.78, 94.79, 94.8, 94.81, 94.82, 94.83, 94.84, 94.85, 94.86, 94.87, 94.88, 94.89, 94.9, 94.91, 94.92, 94.93, 94.94, 94.95, 94.96, 94.97, 94.98, 94.99, 95, 95.01, 95.02, 95.03, 95.04, 95.05, 95.06, 95.07, 95.08, 95.09, 95.1, 95.11, 95.12, 95.13, 95.14, 95.15, 95.16, 95.17, 95.18, 95.19, 95.2, 95.21, 95.22, 95.23, 95.24, 95.25, 95.26, 95.27, 95.28, 95.29, 95.3, 95.31, 95.32, 95.33, 95.34, 95.35, 95.36, 95.37, 95.38, 95.39, 95.4, 95.41, 95.42, 95.43, 95.44, 95.45, 95.46, 95.47, 95.48, 95.49, 95.5, 95.51, 95.52, 95.53, 95.54, 95.55, 95.56, 95.57, 95.58, 95.59, 95.6, 95.61, 95.62, 95.63, 95.64, 95.65, 95.66, 95.67, 95.68, 95.69, 95.7, 95.71, 95.72, 95.73, 95.74, 95.75, 95.76, 95.77, 95.78, 95.79, 95.8, 95.81, 95.82, 95.83, 95.84, 95.85, 95.86, 95.87, 95.88, 95.89, 95.9, 95.91, 95.92, 95.93, 95.94, 95.95, 95.96, 95.97, 95.98, 95.99, 96, 96.01, 96.02, 96.03, 96.04, 96.05, 96.06, 96.07, 96.08, 96.09, 96.1, 96.11, 96.12, 96.13, 96.14, 96.15, 96.16, 96.17, 96.18, 96.19, 96.2, 96.21, 96.22, 96.23, 96.24, 96.25, 96.26, 96.27, 96.28, 96.29, 96.3, 96.31, 96.32, 96.33, 96.34, 96.35, 96.36, 96.37, 96.38, 96.39, 96.4, 96.41, 96.42, 96.43, 96.44, 96.45, 96.46, 96.47, 96.48, 96.49, 96.5, 96.51, 96.52, 96.53, 96.54, 96.55, 96.56, 96.57, 96.58, 96.59, 96.6, 96.61, 96.62, 96.63, 96.64, 96.65, 96.66, 96.67, 96.68, 96.69, 96.7, 96.71, 96.72, 96.73, 96.74, 96.75, 96.76, 96.77, 96.78, 96.79, 96.8, 96.81, 96.82, 96.83, 96.84, 96.85, 96.86, 96.87, 96.88, 96.89, 96.9, 96.91, 96.92, 96.93, 96.94, 96.95, 96.96, 96.97, 96.98, 96.99, 97, 97.01, 97.02, 97.03, 97.04, 97.05, 97.06, 97.07, 97.08, 97.09, 97.1, 97.11, 97.12, 97.13, 97.14, 97.15, 97.16, 97.17, 97.18, 97.19, 97.2, 97.21, 97.22, 97.23, 97.24, 97.25, 97.26, 97.27, 97.28, 97.29, 97.3, 97.31, 97.32, 97.33, 97.34, 97.35, 97.36, 97.37, 97.38, 97.39, 97.4, 97.41, 97.42, 97.43, 97.44, 97.45, 97.46, 97.47, 97.48, 97.49, 97.5, 97.51, 97.52, 97.53, 97.54, 97.55, 97.56, 97.57, 97.58, 97.59, 97.6, 97.61, 97.62, 97.63, 97.64, 97.65, 97.66, 97.67, 97.68, 97.69, 97.7, 97.71, 97.72, 97.73, 97.74, 97.75, 97.76, 97.77, 97.78, 97.79, 97.8, 97.81, 97.82, 97.83, 97.84, 97.85, 97.86, 97.87, 97.88, 97.89, 97.9, 97.91, 97.92, 97.93, 97.94, 97.95, 97.96, 97.97, 97.98, 97.99, 98, 98.01, 98.02, 98.03, 98.04, 98.05, 98.06, 98.07, 98.08, 98.09, 98.1, 98.11, 98.12, 98.13, 98.14, 98.15, 98.16, 98.17, 98.18, 98.19, 98.2, 98.21, 98.22, 98.23, 98.24, 98.25, 98.26, 98.27, 98.28, 98.29, 98.3, 98.31, 98.32, 98.33, 98.34, 98.35, 98.36, 98.37, 98.38, 98.39, 98.4, 98.41, 98.42, 98.43, 98.44, 98.45, 98.46, 98.47, 98.48, 98.49, 98.5, 98.51, 98.52, 98.53, 98.54, 98.55, 98.56, 98.57, 98.58, 98.59, 98.6, 98.61, 98.62, 98.63, 98.64, 98.65, 98.66, 98.67, 98.68, 98.69, 98.7, 98.71, 98.72, 98.73, 98.74, 98.75, 98.76, 98.77, 98.78, 98.79, 98.8, 98.81, 98.82, 98.83, 98.84, 98.85, 98.86, 98.87, 98.88, 98.89, 98.9, 98.91, 98.92, 98.93, 98.94, 98.95, 98.96, 98.97, 98.98, 98.99, 99, 99.01, 99.02, 99.03, 99.04, 99.05, 99.06, 99.07, 99.08, 99.09, 99.1, 99.11, 99.12, 99.13, 99.14, 99.15, 99.16, 99.17, 99.18, 99.19, 99.2, 99.21, 99.22, 99.23, 99.24, 99.25, 99.26, 99.27, 99.28, 99.29, 99.3, 99.31, 99.32, 99.33, 99.34, 99.35, 99.36, 99.37, 99.38, 99.39, 99.4, 99.41, 99.42, 99.43, 99.44, 99.45, 99.46, 99.47, 99.48, 99.49, 99.5, 99.51, 99.52, 99.53, 99.54, 99.55, 99.56, 99.57, 99.58, 99.59, 99.6, 99.61, 99.62, 99.63, 99.64, 99.65, 99.66, 99.67, 99.68, 99.69, 99.7, 99.71, 99.72, 99.73, 99.74, 99.75, 99.76, 99.77, 99.78, 99.79, 99.8, 99.81, 99.82, 99.83, 99.84, 99.85, 99.86, 99.87, 99.88, 99.89, 99.9, 99.91, 99.92, 99.93, 99.94, 99.95, 99.96, 99.97, 99.98, 99.99, or 100 mg/day, or any amount below, above, or in between these values.

In some embodiments, the capsule can be in physical contact with the foreign object. In other examples, the abnormal capsule can be within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 centimeters of the foreign object.

### EXAMPLES

### Example 1: Tamoxifen Reduces Breast Implant Capsule Formation in a Mouse Model

### Methods

### Animal Care and Surgical Implantation

All procedures and experiments involving the use of animals in this study were done in accordance with relevant guidelines and regulations with approval from the Abigail Wexner Research Institute (AWRI) at Nationwide Children's Hospital Institutional Animal Care and Use Committee (IACUC) (Protocol AR12-00075).

Eight-week-old C57BL/6 or Beige (C57BL/6J-Lyst^{bg-J}/J) female mice (Jackson Labs, Bar Harbor, ME, USA) (N=10/group) were treated with daily tamoxifen or vehicle injections into the intraperitoneal cavity 1 week before surgery. Tamoxifen injections consisted of 75 mg/kg tamoxifen citrate (Sigma, St. Louis MO, USA) in peanut oil (Sigma, St Louis MO, USA) with 10% ethanol (Sigma, St. Louis MO, USA) to promote dissolution of tamoxifen, while control injections consisted of only peanut oil and ethanol. Daily injections continued until the day of explantation, two weeks after implantation.

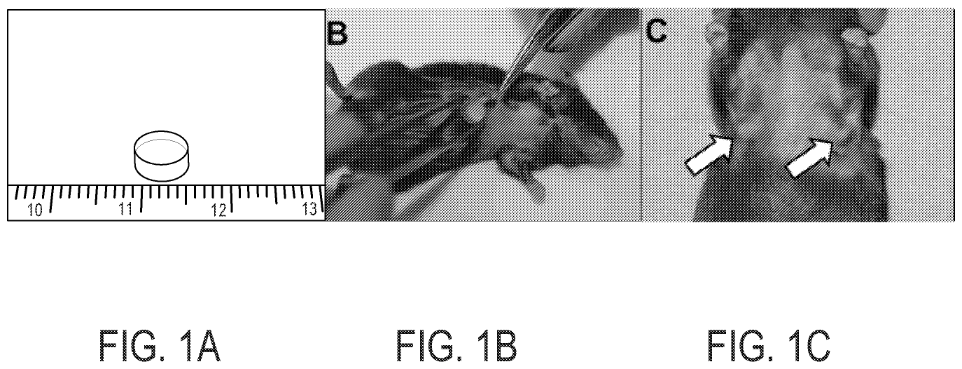

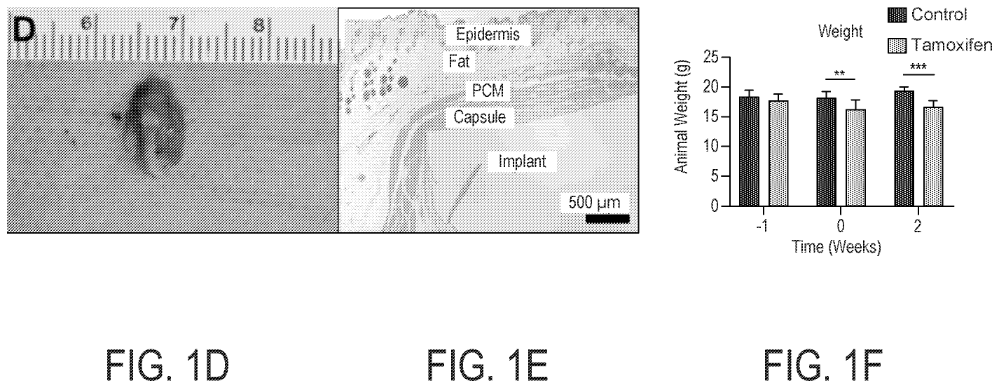

Silicone prostheses were cut from a solid sheet of medical grade silicone (Goodfellow, Coraopolis, PA, USA) using a 6 mm biopsy punch (FIG. 1A). Prostheses were sterilized using 70% ethanol.

Mice were anesthetized with a standard ketamine/xylazine cocktail. Fur was clipped and skin was prepped with betadine followed by alcohol pads. Silicone prostheses were implanted in the submammary plane, bilaterally, giving a total of 20 implants per experimental group (FIG. 1B). Skin incisions were re-approximated with 6-0 nylon suture (FIG. 1C).

After 2 weeks, mice were euthanized and the silicone prostheses were removed en bloc with all surrounding tissue, including the overlying skin for orientation (FIG. 1D). Some animals accessed their incision causing implant exposure. All implant exposures occurred within 48 hours after implantation. These samples were excluded from further analysis owing to the potential confounding nature of bacterial contamination on capsule formation.

### Histology

Explanted silicone pieces and surrounding capsules were fixed overnight in formaldehyde at 4° C. and transitioned to 70% ethanol. Samples were then paraffin embedded and sectioned at 4 μm. Samples were stained with H&E (FIG. 1E) or Picro-Sirius Red. Immunohistochemistry sections were deparaffinized, rehydrated and blocked for endogenous peroxidase activity (3% H2O2 in H2O) and nonspecific background staining (3% normal goat serum in Background Sniper, BioCare Medical, CA, USA). Antigen retrieval was performed with citrate buffer (pH 6.0) or Tris-EDTA (pH 9.0) in a pressure cooker for 10 minutes and slides were incubated for 30 minutes at room temperature with primary antibodies. Primary antibodies (all from Abcam, Cambridge, UK) included α-SMA (ab124964), CD31 (ab28364), CD68 (ab125212), iNOS (ab15323), CD163 (ab182422), Ki67 (ab15580), ER-α (ab271827), and ER-β (ab3576). Primary antibody binding was detected by subsequent incubation with species appropriate biotinylated IgG (Vector Laboratories, CA, USA), followed by streptavidin-horse radish peroxidase (Vector Laboratories, CA, USA) and chromogenic development with 3,3-diaminobenzidine (Vector Laboratories, CA, USA). Tissue sections were counterstained with Gill's hematoxylin (Vector Laboratories, CA, USA), slides were dehydrated and a cover slip was placed. Slides were imaged on a Zeiss Axio Observer Z1 inverted microscope (Zeiss, Germany) and quantified using ImageJ (NIH, USA).

Histology was quantified from H&E slides within the fibrous capsule on the side oriented with overlying skin to maintain consistency and remove potential variability by dissection at explantation. Due to the substantial differences in total capsular area, cellular histological stains were quantified using two methodologies, both as a percent of total cells within the capsule to determine differences in make-up of the capsular tissue, and as cells/mm of implant surface to determine differences in total amount of each cell type present.

### Statistics

All statistical analysis was performed in Prism 8.0 (GraphPad, San Diego, CA, USA). Data was evaluated for outliers using the Grubb's test. Control and tamoxifen-treated groups were compared with unpaired t-tests, except in cases where the equal variance assumption was violated; when the nonparametric Mann-Whitney test was utilized. A p-value of 0.05 was considered significant.

### Results

### Surgery and Follow-Up

After exclusion of implants that were exposed after surgery, 8/20 implants were available for the control group and 15/20 for the tamoxifen-treated group for further analysis. While control and tamoxifen-treated groups had similar weights at the start of the injection period (18.2±1.2 control vs 17.6±1.2 tamoxifen gram body weight, p=0.279 unpaired t-test), the tamoxifen-treated animals had a lower body weight one week later at the date of surgery (18.2±1.0 control vs 16.2±1.6 tamoxifen gram body weight, p<0.01 unpaired t-test) and at the two-week explant date (19.4±0.5 control vs 16.6±1.0 tamoxifen gram body weight, p<0.001 unpaired t-test; FIG. 1F).

### Capsule Thickness and Cellular Infiltration

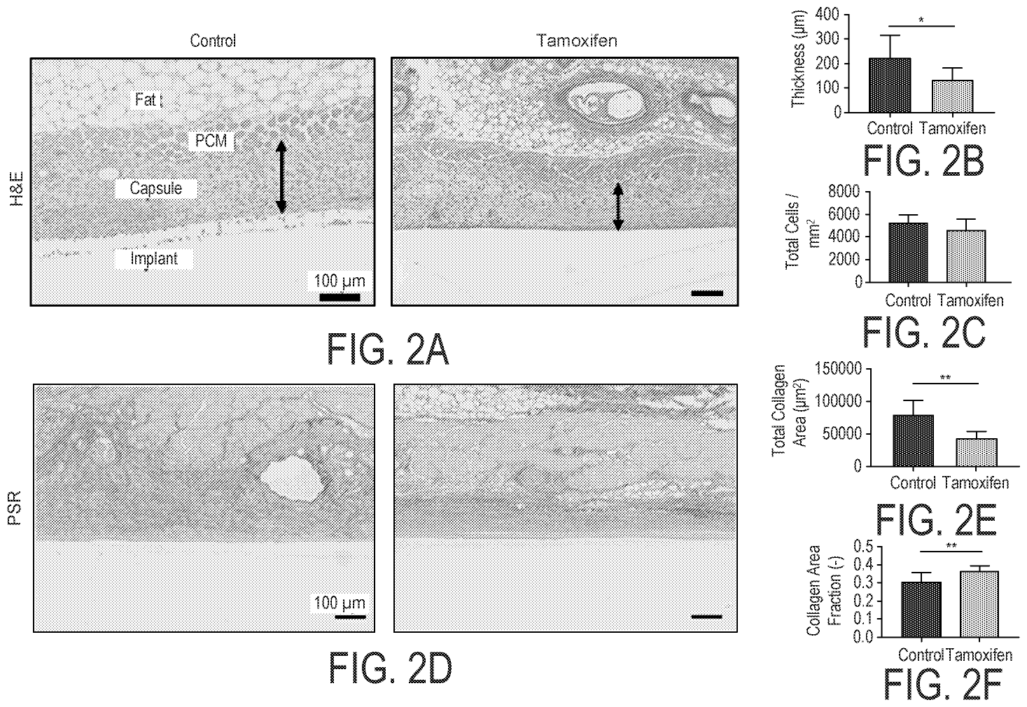

Capsules were identified by histology as the plane of tissue formed between the implant surface and the panniculus carnosus muscle (FIG. 2A). Implant capsules were significantly thinner in the tamoxifen-treated cohort (222±89 μm control vs 132±49 μm tamoxifen, p=0.0159 Mann-Whitney test; FIG. 2B), demonstrating a 59% reduction in capsule thickness. Cellularity of the capsules was found to be similar (3634±398 cells/mm² control vs 3624±608 cells/mm² tamoxifen, p=0.9684 unpaired t-test; FIG. 2C).

### Collagen Deposition

Total collagen surrounding the implants (FIG. 2D) was significantly lower in the tamoxifen treated animals compared to the untreated controls (79312±22991 μm² control vs 43598±10044 μm² tamoxifen, p=0.001 Mann-Whitney test; FIG. 2E). However, due to the differences in thickness between the capsules, there was a significantly higher area percent of collagen in the tamoxifen treated group (30.4±5.8% control vs 36.2±3.1% tamoxifen, p=0.023 Mann-Whitney test; FIG. 2F).

### Estrogen Receptors

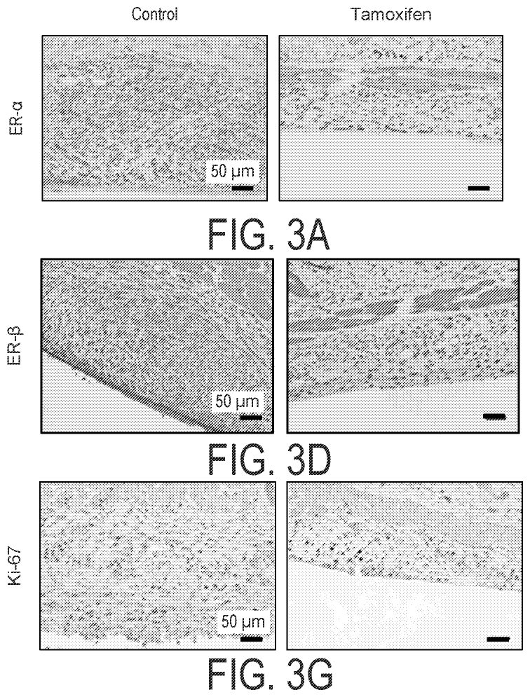

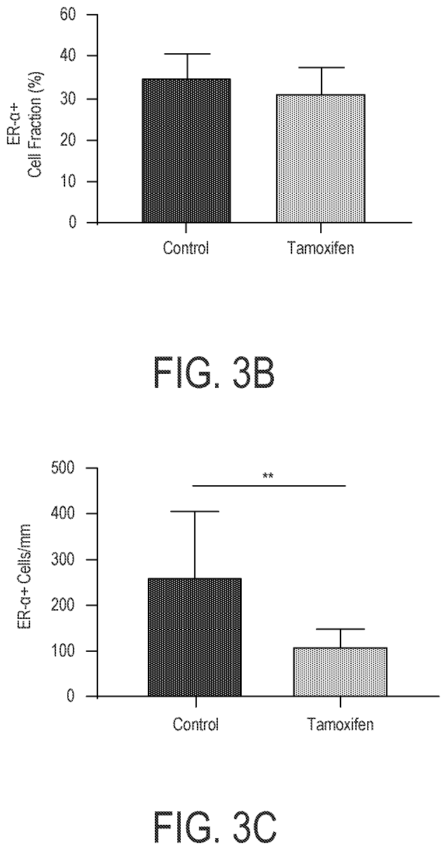

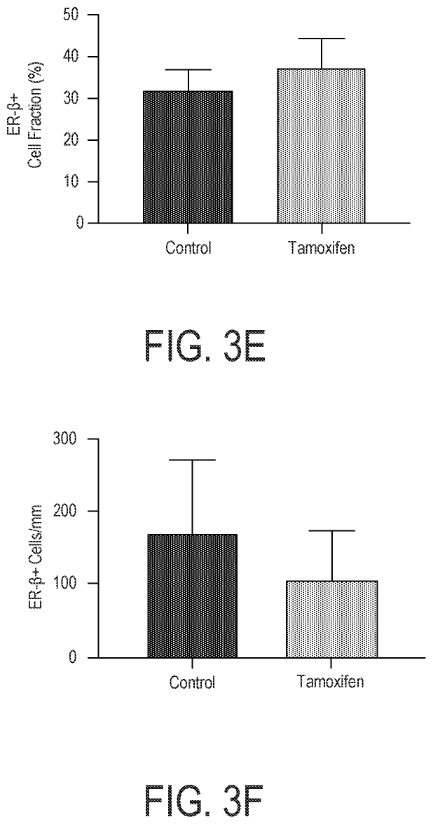

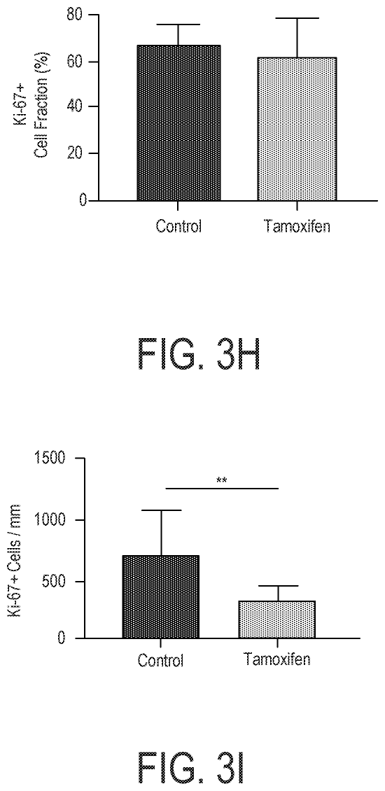

Estrogen receptor α (ER-α) and estrogen receptor β (ER-β) were investigated, as tamoxifen is an estrogen analog that functions primarily through these targets. ER-α expression (FIG. 3A) as a fraction of total cells was not affected by tamoxifen treatment (34.7±5.8% control vs 31.0±6.3% tamoxifen, p=0.184 unpaired t-test; FIG. 3B), but was lowered as a total cells/mm of capsular tissue (256.5±151.2 cells/mm control vs 107.0±39.8 cells/mm tamoxifen, p=0.003 Mann-Whitney test; FIG. 3C). ER-β expression (FIG. 3D) was not affected as measured by cellular fraction (31.6±5.0% control vs 36.8±7.5% tamoxifen, p=0.092 unpaired t-test; FIG. 3E) nor total cells/mm (169.3±102.9 cells/mm control vs 106.2±69.2 cells/mm tamoxifen, p=0.094 unpaired t-test; FIG. 3F).

### Cellular Proliferation

Ki-67 was used as a marker of cellular proliferation (FIG. 3G). The cellular fraction of Ki-67+ cells was similar between groups (67.5±9.0% control vs 62.4±16.9% tamoxifen, p=0.440 unpaired t-test; FIG. 3H); however there were significantly fewer Ki-67+ cells/mm in the tamoxifen treated mice (704±366 cells/mm control vs 324±125 cells/mm tamoxifen, p=0.003 Mann-Whitney test; FIG. 3I).

### Myofibroblasts

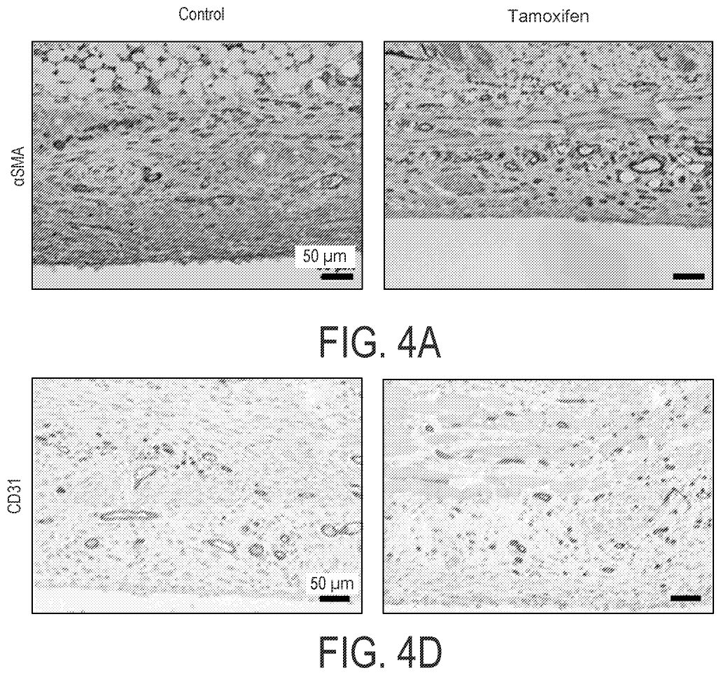

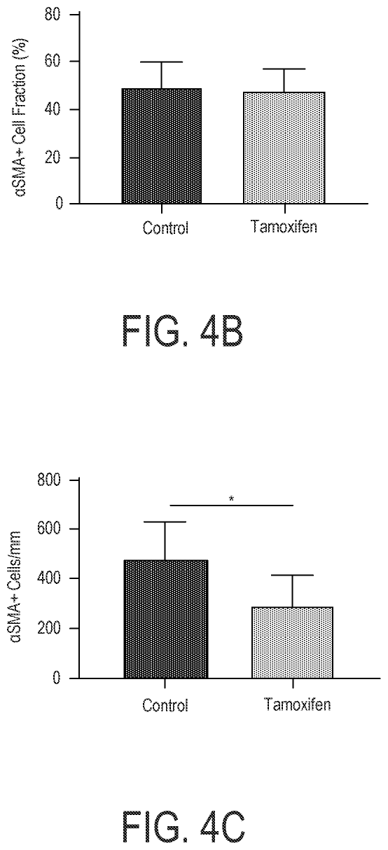

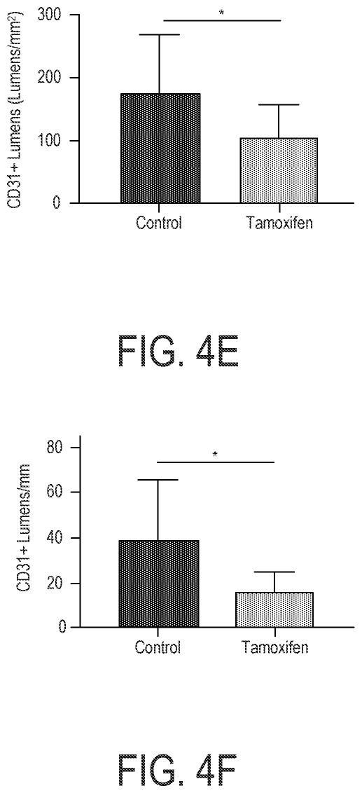

Alpha smooth muscle actin (αSMA) was utilized as a marker of myofibroblasts within the implant capsule (FIG. 4A). Although a similar percentage of total cells αSMA+ (49.6±10.6% control vs 47.9±9.5% tamoxifen, p=0.690 unpaired t-test; FIG. 4B), the linear density of αSMA+cells was significantly reduced in tamoxifen treated animals relative to controls (477±156 cells/mm control vs 295±121 cells/mm tamoxifen, p=0.005 unpaired t-test; FIG. 4C).

### Neovascularization

CD31, a marker of endothelial cells, was used to identify the formation of blood vessels within the capsule (FIG. 4D). Tamoxifen treated mice were found to have significantly fewer vessels per mm² than control animals (173.9±96.1 vessels/mm² control vs 106.3±51.8 vessels/mm² tamoxifen, p=0.043 unpaired t-test; FIG. 4E) and fewer total CD31+vessels (39.1±26.7 vessels/mm control vs 15.9±9.7 vessels/mm, p=0.040 Mann-Whitney test; FIG. 4F).

### Macrophages

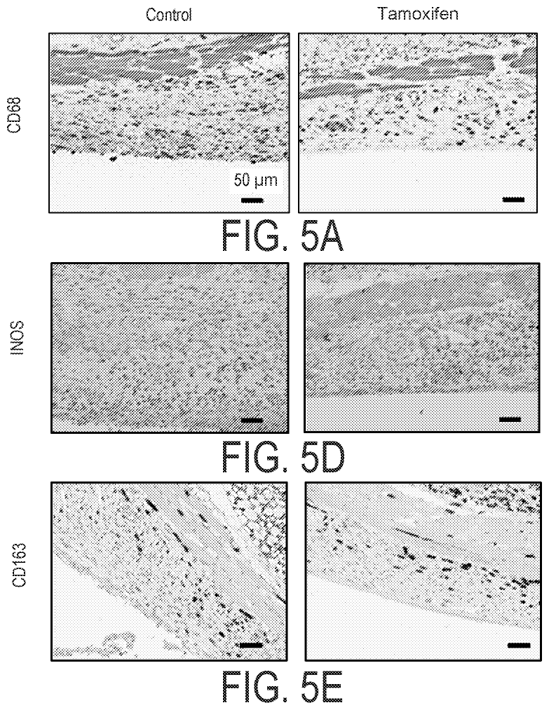

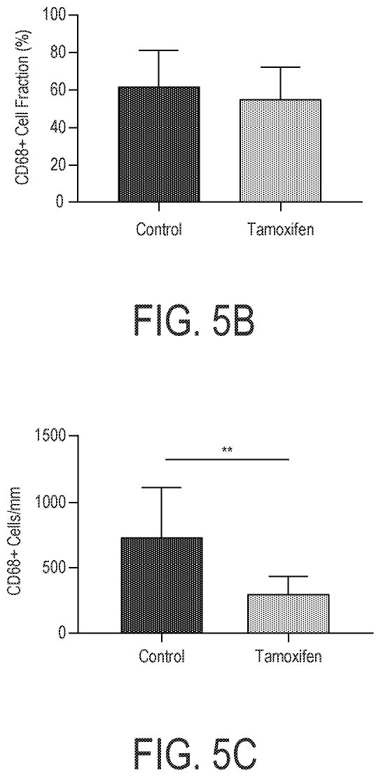

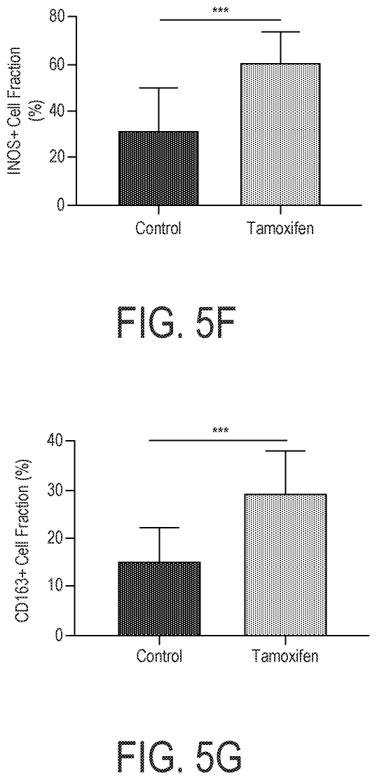

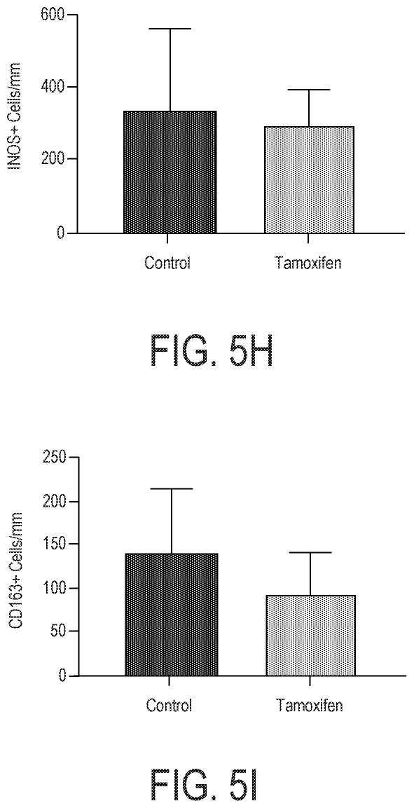

Macrophage infiltration of the capsule was investigated using CD68 (FIG. 5A). There was not a difference in cellular fraction of CD68 (62.1±19.1% control vs 55.9±16.0% tamoxifen, p=0.419 unpaired t-test; FIG. 5B). The linear density of CD68+ cells surrounding the tamoxifen implants was lower than the control implants (718.9±391.2 cells/mm control vs 303.3±136.5 cells/mm, p=0.011 Mann-Whitney test; FIG. 5C).

Pro-inflammatory and anti-inflammatory macrophages were labeled with iNOS (FIG. 5D) and CD163 (FIG. 5E), respectively. The cellular fractions of both iNOS and CD163 were higher in tamoxifen treated animals (iNOS 31.6±18.1% control vs 60.6±12.6% tamoxifen, p<0.001 unpaired t-test; FIG. 5F and CD163 14.7±7.2% control vs 29.1±8.4% tamoxifen, p<0.001 unpaired t-test; FIG. 5G). There were no differences seen in the total number of cells with positivity for iNOS or CD163 between groups (iNOS 336.1±226.3 cells/mm control vs 290.6±104.2 cells/mm tamoxifen, p>0.999 Mann-Whitney test; FIG. 5H, and CD163 136.6±76.4 cells/mm control vs 94.1±45.9 cells/mm tamoxifen, p=0.108 unpaired t-test; FIG. 5I).

### Effect of Beige Mutation on Capsular Formation

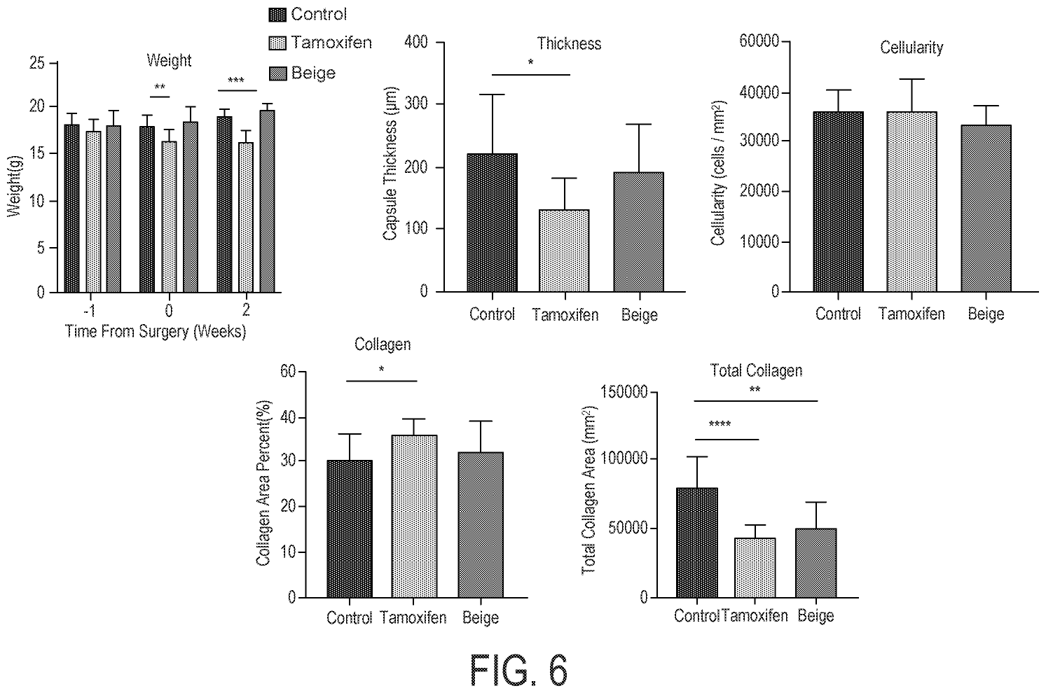

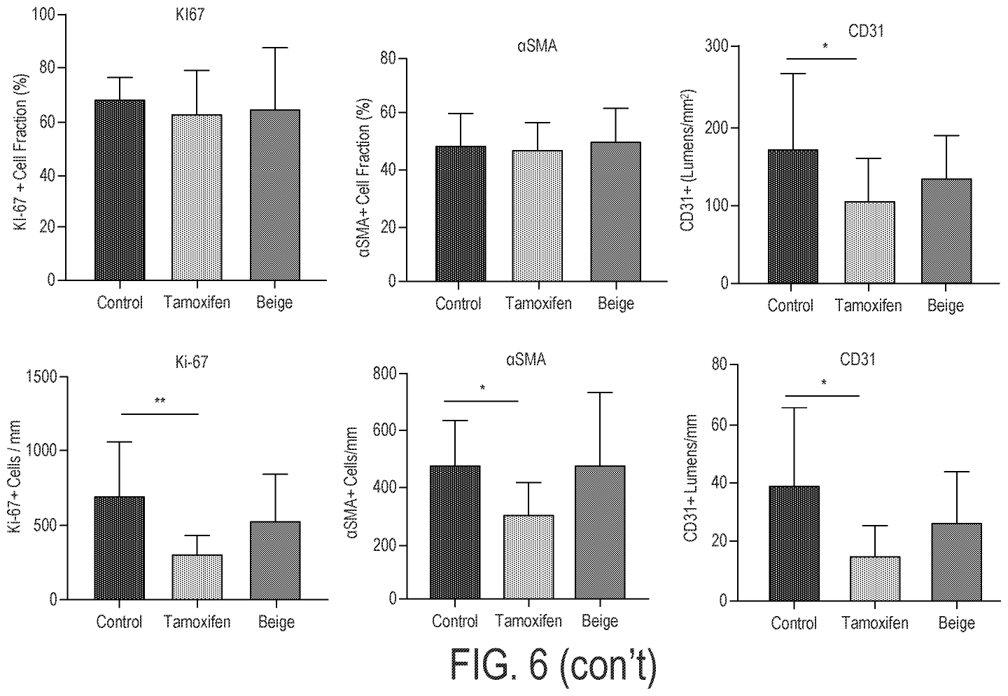

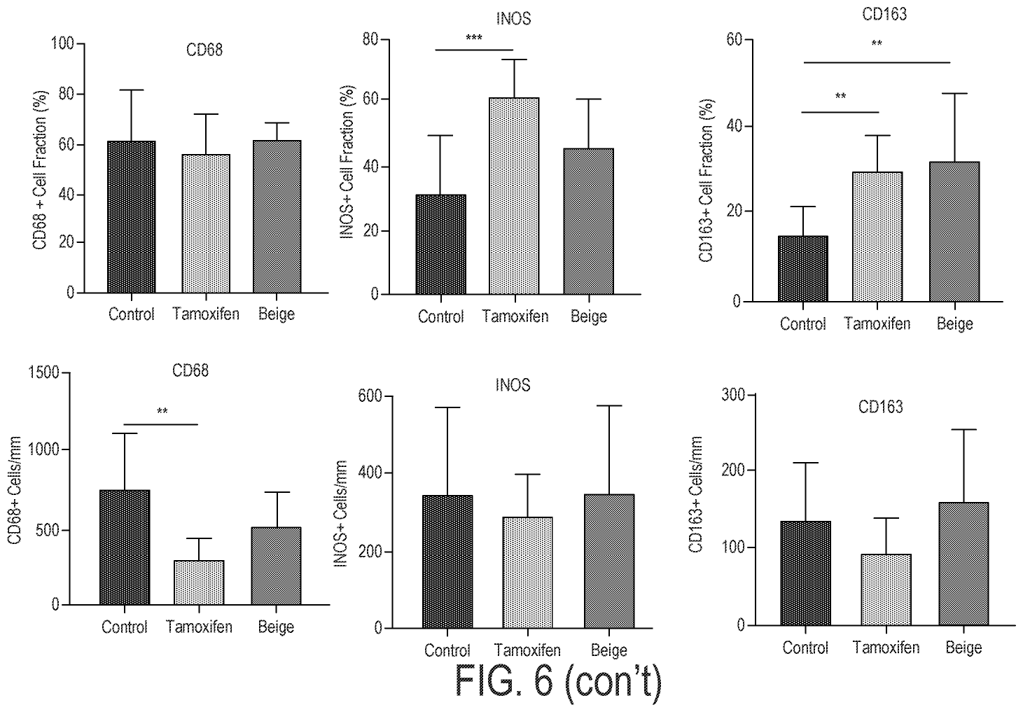

The effects of innate immunodeficiency was compared to tamoxifen treatment to delineate potential contributions of immune modulation versus anti-proliferative effects of tamoxifen. The beige mouse was used as a model of innate immunodeficiency (Hibino 2015; Zbinden 2020; Spritz 1998). Beige mice were similarly treated with vehicle injections identical to control animals. Similar to the control and tamoxifen-treated groups, 10 out of 20 beige implants were removed from study due to implant exposure over the first 48 hours post-implantation. Beige mice had comparable capsule thickness to control mice (222±95 μm control vs 190±78 μm beige, p=0.436 unpaired t-test.). Most histological markers did not exhibit differences between control and beige animals; however there was a decreased amount of collagen deposition in beige animals compared to controls (79312±22991 μm² control vs 51400±16951 μm² beige, p=0.016 Mann-Whitney test; FIG. 6). Interestingly, there were no statistical differences between the beige animals and the tamoxifen treated animals in any histologic markers. Overall, the beige model appeared to have a minor trend toward decreasing fibrous capsule formation, but to a lesser extent than tamoxifen treatment.

### Discussion

This study demonstrates the potential utility of tamoxifen for reducing fibrous capsule formation in a mouse model of silicone breast implantation. Total fibrous capsule thickness and collagen deposition were significantly reduced in tamoxifen treated mice relative to controls. Due to differences in total tissue formed around implants of identical size, comparison of total cells encasing the implant as well as the average composition of the deposited tissue for each marker studied was crucial for understanding the noted histological differences.

As disclosed herein, tamoxifen behaved as a potent anti-proliferative or anti-cellular-infiltrative agent to reduce capsule formation around a foreign body, and this effect appeared to be more contributory than immunomodulation. Tamoxifen is a known anti-proliferative agent in estrogen receptor positive breast cancer, in other tumors, such as desmoid tumors, and fibrosing conditions (Gragnani 2010; Bardon 1984; Hansmann 2004). In rats, tamoxifen has been shown to decrease fibroblast viability, proliferation, epidural fibrosis and dural thickness following laminectomy (Wang 2018; Ozturk 2018). Tamoxifen also acts as an anti-fibrotic treatment for patients with idiopathic retroperitoneal fibrosis (Ergun 2005; van der Bilt 2016). Additionally, tamoxifen has a potent anti-angiogenic effect (Bogush 2012; Helmestam 2012). These findings are relevant because higher Baker grades of capsular contracture are associated with greater levels of angiogenesis (Segreto 2018).

While the total numbers of CD68+cells were decreased in tamoxifen treated mice, total amounts of both pro-inflammatory iNOS+and anti-inflammatory CD163+ macrophages were not affected. This difference can be explained by the promiscuity of the CD68 cellular marker, which stains not only macrophages, but also fibroblasts (Gottfried 2008; Inoue 2005). Indeed, surprisingly, myofibroblasts, denoted by αSMA, were decreased in tamoxifen treatment, and can explain the differences in CD68 staining. These results show that tamoxifen modulates the fibrous capsule formation by directly inhibiting the infiltration of fibroblastic cells and total deposition of collagen, without a significant effect on the inflammatory cell response. This is further supported by the fact that there was little modulation of capsule formation when the same technique was applied to a mouse model of innate immunodeficiency, the beige mouse.

In this study, tamoxifen treatment was shown to have an effect on ER-α expression, but not on ER-β expression. Tamoxifen's primary target is ER-α, with additional relatively low interaction with ER-β. Tamoxifen has mechanisms of action both directly through the hormonal estrogen receptors, as well as non-hormonal pathways. It is important to recognize that the hormonal effects of estrogen, as well as estrogen analogs such as tamoxifen, are tissue specific. Non-hormonal mechanisms of action of tamoxifen are believed to act through the TGF-β pathway. These mechanisms of action are important considerations in the context of these surprising findings. Importantly, systemic treatment with tamoxifen and its associated side effects can be avoided through the use of local release of tamoxifen and its active metabolite endoxifen, which has shown 30- to 100-fold greater potency than tamoxifen when delivered orally or through IV injections in mice (Reid 2014).

The compositions, devices, systems, and methods of the appended claims are not limited in scope by the specific compositions, devices, systems, and methods described herein, which are intended as illustrations of a few aspects of the claims. Any compositions, devices, systems, and methods that are functionally equivalent are intended to fall within the scope of the claims. Various modifications of the compositions, devices, systems, and methods in addition to those shown and described herein are intended to fall within the scope of the appended claims. Further, while only certain representative compositions, devices, systems, and method steps disclosed herein are specifically described, other combinations of the compositions, devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

Unless defined otherwise, all technical and scientific terms used herein have the same meanings as commonly understood by one of skill in the art to which the disclosed invention belongs. Publications cited herein and the materials for which they are cited are specifically incorporated by reference.

### REFERENCES

1. Bui JM, Perry T, Ren CD, Nofrey B, Teitelbaum S, Van Epps DE. Histological characterization of human breast implant capsules. Aesthetic Plast Surg. 2015;39(3):306-15.
2. Anderson JM, Rodriguez A, Chang DT. Foreign body reaction to biomaterials. Semin Immunol. 2008;20(2):86-100.
3. Segreto F, Carotti S, Marangi GF, Tosi D, Zingariello M, Pendolino AL, et al. The role of angiogenesis, inflammation and estrogen receptors in breast implant capsules development and remodeling. J Plast Reconstr Aesthet Surg. 2018;71(5):637-43.
4. Adams WP, Haydon MS, Raniere J, Trott S, Marques M, Feliciano M, et al. A rabbit model for capsular contracture: development and clinical implications. Plast Reconstr Surg. 2006;117(4):1214-9; discussion 20-1.
5. Shin BH, Kim BH, Kim S, Lee K, Choy YB, Heo CY. Silicone breast implant modification review: overcoming capsular contracture. Biomater Res. 2018;22:37.
6. Spear SL, Baker JL. Classification of capsular contracture after prosthetic breast reconstruction. Plast Reconstr Surg. 1995;96(5):1119-23; discussion 24.
7. Wan D, Rohrich RJ. Revisiting the Management of Capsular Contracture in Breast Augmentation: A Systematic Review. Plast Reconstr Surg. 2016;137(3):826-41.
8. Mempin M, Hu H, Chowdhury D, Deva A, Vickery K. The A, B and C's of Silicone Breast Implants: Anaplastic Large Cell Lymphoma, Biofilm and Capsular Contracture. Materials (Basel). 2018;11(12).
9. Haran O, Bracha G, Tiosano A, Menes T, Madah E, Gur E, et al. Postirradiation Capsular Contracture in Implant-Based Breast Reconstruction: Management and Outcome. Plast Reconstr Surg. 2021;147(1):11-9.
10. Headon H, Kasem A, Mokbel K. Capsular Contracture after Breast Augmentation: An Update for Clinical Practice. Arch Plast Surg. 2015;42(5):532-43.
11. Fischer S, Hirche C, Reichenberger MA, Kiefer J, Diehm Y, Mukundan S, et al. Silicone Implants with Smooth Surfaces Induce Thinner but Denser Fibrotic Capsules Compared to Those with Textured Surfaces in a Rodent Model. PLoS One. 2015;10(7):e0132131.
12. Coleman DJ, Foo IT, Sharpe DT. Textured or smooth implants for breast augmentation? A prospective controlled trial. Br J Plast Surg. 1991;44(6):444-8.
13. Hwang MJ, Brown H, Murrin R, Momtahan N, Sterne GD. Breast implant-associated anaplastic large cell lymphoma: a case report and literature review. Aesthetic Plast Surg. 2015;39(3):391-5.
14. Chen VW, Hoang D, Clancy S. Breast Implant-Associated Bilateral B-Cell Lymphoma. Aesthet Surg J. 2020;40(2):NP52-NP8.
15. Ajdic D, Zoghbi Y, Gerth D, Panthaki ZJ, Thaller S. The Relationship of Bacterial Biofilms and Capsular Contracture in Breast Implants. Aesthet Surg J. 2016;36(3):297-309.
16. Persichetti P, Segreto F, Carotti S, Marangi GF, Tosi D, Morini S. Oestrogen receptor-alpha and -beta expression in breast implant capsules: experimental findings and clinical correlates. J Plast Reconstr Aesthet Surg. 2014;67(3):308-15.
17. Bae HS, Son HY, Lee JP, Chang H, Park JU. The Role of Periostin in Capsule Formation on Silicone Implants. Biomed Res Int. 2018;2018:3167037.
18. Kelley BP, Valero V, Yi M, Kronowitz SJ. Tamoxifen increases the risk of microvascular flap complications in patients undergoing microvascular breast reconstruction. Plast Reconstr Surg. 2012;129(2):305-14.
19. Mousavi SR, Raaiszadeh M, Aminseresht M, Behjoo S. Evaluating tamoxifen effect in the prevention of hypertrophic scars following surgical incisions. Dermatol Surg. 2010;36(5):665-9.
20. Payne WG, Ko F, Anspaugh S, Wheeler CK, Wright TE, Robson MC. Down-regulating causes of fibrosis with tamoxifen: a possible cellular/molecular approach to treat rhinophyma. Ann Plast Surg. 2006;56(3):301-5.
21. Gragnani A, Warde M, Furtado F, Ferreira LM. Topical tamoxifen therapy in hypertrophic scars or keloids in burns. Arch Dermatol Res. 2010;302(1):1-4.
22. Delle H, Rocha JR, Cavaglieri RC, Vieira JM, Malheiros DM, Noronha IL. Antifibrotic effect of tamoxifen in a model of progressive renal disease. J Am Soc Nephrol. 2012;23(1):37-48.
23. Hibino N, Mejias D, Pietris N, Dean E, Yi T, Best C, et al. The innate immune system contributes to tissue-engineered vascular graft performance. FASEB J. 2015;29(6):2431-8.
24. Zbinden JC, Blum KM, Berman AG, Ramachandra AB, Szafron JM, Kerr KE, et al. Effects of Braiding Parameters on Tissue Engineered Vascular Graft Development. Adv Healthc Mater. 2020;9(24):e2001093.
25. Spritz RA. Genetic defects in Chediak-Higashi syndrome and the beige mouse. J Clin Immunol. 1998;18(2):97-105.
26. Bardon S, Vignon F, Derocq D, Rochefort H. The antiproliferative effect of tamoxifen in breast cancer cells: mediation by the estrogen receptor. Mol Cell Endocrinol. 1984;35(2-3):89-96.
27. Kinzbrunner B, Ritter S, Domingo J, Rosenthal CJ. Remission of rapidly growing desmoid tumors after tamoxifen therapy. Cancer. 1983;52(12):2201-4.
28. Hansmann A, Adolph C, Vogel T, Unger A, Moeslein G. High-dose tamoxifen and sulindac as first-line treatment for desmoid tumors. Cancer. 2004;100(3):612-20.
29. Wang S, Li X, Yan L, Nie Q, Dai J, Chen H, et al. Tamoxifen inhibits fibroblast proliferation and prevents epidural fibrosis by regulating the AKT pathway in rats. Biochem Biophys Res Commun. 2018;497(4):937-42.
30. Ozturk Y, Bozkurt I, Yaman ME, Guvenc Y, Tolunay T, Bayram P, et al. Histopathologic Analysis of Tamoxifen on Epidural Fibrosis. World Neurosurg. 2018;111:e941-e8.
31. Ergun I, Keven K, Canbakan B, Ekmekci Y, Erbay B. Tamoxifen in the treatment of idiopathic retroperitoneal fibrosis. Int Urol Nephrol. 2005;37(2):341-3.
32. van der Bilt FE, Hendriksz TR, van der Meijden WA, Brilman LG, van Bommel EF. Outcome in patients with idiopathic retroperitoneal fibrosis treated with corticosteroid or tamoxifen monotherapy. Clin Kidney J. 2016;9(2):184-91.
33. Bogush T, Dudko E, Bogush E, Polotsky B, Tjulandin S, Davydov M. Tamoxifen non-estrogen receptor mediated molecular targets. Oncol Rev. 2012;6(2):e15.
34. Helmestam M, Andersson H, Stavreus-Evers A, Brittebo E, Olovsson M. Tamoxifen modulates cell migration and expression of angiogenesis-related genes in human endometrial endothelial cells. Am J Pathol. 2012;180(6):2527-35.
35. Gottfried E, Kunz-Schughart LA, Weber A, Rehli M, Peuker A, Müller A, et al. Expression of CD68 in non-myeloid cell types. Scand J Immunol. 2008;67(5):453-63.
36. Inoue T, Plieth D, Venkov CD, Xu C, Neilson EG. Antibodies against macrophages that overlap in specificity with fibroblasts. Kidney Int. 2005;67(6):2488-93.
37. Gruvberger-Saal SK, Bendahl PO, Saal LH, Laakso M, Hegardt C, Edén P, et al. Estrogen receptor beta expression is associated with tamoxifen response in ERalpha-negative breast carcinoma. Clin Cancer Res. 2007;13(7):1987-94.
38. Xue M, Zhang K, Mu K, Xu J, Yang H, Liu Y, et al. Regulation of estrogen signaling and breast cancer proliferation by an ubiquitin ligase TRIM56. Oncogenesis. 2019;8(5):30.
39. Bentzen SM, Skoczylas JZ, Overgaard M, Overgaard J. Radiotherapy-related lung fibrosis enhanced by tamoxifen. J Natl Cancer Inst. 1996;88(13):918-22.
40. Nishino M, Hayakawa K, Nakamura Y, Morimoto T, Mukaihara S. Effects of tamoxifen on hepatic fat content and the development of hepatic steatosis in patients with breast cancer: high frequency of involvement and rapid reversal after completion of tamoxifen therapy. AJR Am J Roentgenol. 2003;180(1):129-34.
41. Chen H, Tritton TR, Kenny N, Absher M, Chiu JF. Tamoxifen induces TGF-beta 1 activity and apoptosis of human MCF-7 breast cancer cells in vitro. J Cell Biochem. 1996;61(1):9-17.
42. Varras M, Polyzos D, Akrivis C. Effects of tamoxifen on the human female genital tract: review of the literature. Eur J Gynaecol Oncol. 2003;24(3-4):258-68.
43. Shehata M, van Amerongen R, Zeeman AL, Giraddi RR, Stingl J. The influence of tamoxifen on normal mouse mammary gland homeostasis. Breast Cancer Res. 2014;16(4):411.
44. Reid JM, Goetz MP, Buhrow SA, Walden C, Safgren SL, Kuffel MJ, et al. Pharmacokinetics of endoxifen and tamoxifen in female mice: implications for comparative in vivo activity studies. Cancer Chemother Pharmacol. 2014;74(6):1271-8.
45. Ng MF. Cachexia - an intrinsic factor in wound healing. Int Wound J. 2010;7(2):107-13.
46. Hunt BJ. Hemostasis at Extremes of Body Weight. Semin Thromb Hemost. 2018;44(7):632-9.
47. Blum K, Roby L, Zbinden J, Chang Y, Mirhaidari G, Reinhardt J, et al. Sex and Tamoxifen Confound Murine Experimental Studies in Cardiovascular Tissue Engineering. Nat Sci Rep; Forthcoming2021.
48. Moon DJ, Deva AK. Adverse Events Associated with Breast Implants: The Role of Bacterial Infection and Biofilm. Clin Plast Surg. 2021;48(1):101-8.
49. Grada A, Mervis J, Falanga V. Research Techniques Made Simple: Animal Models of Wound Healing. J Invest Dermatol. 2018;138(10):2095-105.e1.

1. A method for treating or preventing capsular contracture or capsule formation in a subject with an implant, the method comprising administering to the subject tamoxifen or a derivative, formulation, or metabolite thereof in a localized form.

2. The method of claim 1, wherein said implant is a breast implant; artificial joint or limb; cardiac pacemaker; coronary stent; implantable cardioverter defibrillator; cerebral spinal fluid shunt system; cochlear implant or ear tube; surgical mesh implant; screws, rods, or artificial discs; intrauterine device; artificial eye lens.

3. The method of claim 1, wherein the derivative, formulation, or metabolite of tamoxifen is endoxifen, N-desmethyl-tamoxifen, or Z-4-hydroxy-tamoxifen.

4. The method of claim 1, wherein the tamoxifen, or a derivative, formulation or metabolite thereof is conjugated to the implant, or the implant is soaked in the tamoxifen or associated metabolite, derivative, or formulation thereof.

5. The method of claim 1, wherein the tamoxifen, or a derivative, formulation or metabolite thereof is administered locally near the implant.

6. The method of claim 5, wherein the tamoxifen, or a derivative, formulation or metabolite thereof is administered in a separate device from the implant.

7. The method of claim 1, wherein the tamoxifen, or a derivative, formulation or metabolite thereof is administered in a gel form.

8. The method of claim 1, wherein the tamoxifen, or a derivative, formulation or metabolite thereof is provided in slow-release form.

9. The method of claim 1, wherein the tamoxifen, or a derivative, formulation or metabolite thereof is provided in an amount which is less than the amount given to treat or prevent breast cancer.

10. The method of claim 1, wherein the tamoxifen, or a derivative, formulation or metabolite thereof is provided in an amount which is greater than the amount given to treat or prevent breast cancer.

11. The method of claim 1, wherein, in addition to tamoxifen, or a derivative, formulation or metabolite thereof, the subject is given another drug, formulation, or method of treatment simultaneously.

12. The method of claim 1, wherein the tamoxifen, metabolite, derivative, or formulation thereof can be used with an adjuvant, carrier, or formulation for delivery.

13. A surgical implant, wherein said implant is conjugated to tamoxifen or a metabolite thereof.

14. The surgical implant of claim 13, wherein said implant is a breast implant; artificial joint or limb; cardiac pacemaker; coronary stent; implantable cardioverter defibrillator; cerebral spinal fluid shunt system; cochlear implant or ear tube; surgical mesh implant; screws, rods, or artificial discs; intrauterine device; artificial eye lens.

15. The surgical implant of claim 13, wherein the metabolite of tamoxifen is endoxifen.

16. The surgical implant of claim 13, wherein the tamoxifen or metabolite thereof is conjugated to the implant, or the implant is soaked in the tamoxifen or associated metabolite, derivative, or formulation thereof.

17. The surgical implant of claim 13, wherein the tamoxifen or metabolite thereof is in a gel form.

18. The surgical implant of claim 13, wherein the tamoxifen or metabolite thereof is provided in slow-release form.

19. The surgical implant of claim 13, wherein the tamoxifen or metabolite thereof is provided in an amount which is less than the amount given to treat or prevent breast cancer.

20. The surgical implant of claim 13, wherein the tamoxifen or metabolite thereof is provided in an amount which is greater than the amount given to treat or prevent breast cancer.
